(19) United States
(12) Patent Application Publication
(10) Pub. No.: US 2008/0207694 A1
(43) Pub. Date: Aug. 28, 2008

(54) Hydroxamates as Histone Deacetylase Inhibitors and Pharmaceutical Formulations Containing Them

(76) Inventors: Cristina Rossi, Pomezia (IT); Marina Porcelloni, Abbadia San Salvatore (IT); Piero D'Andrea, Genzano (IT); Daniela Fattori, Velletri (IT); Elena Marastoni, Roma (IT)

Correspondence Address: JAY S CINAMON;ABELMAN, FRAYNE & SCHWAB, 666 THIRD AVENUE, 10TH FLOOR, NEW YORK, NY, 10017-5621, US

(21) Appl. No.: 11/886,160

(22) PCT Filed: Mar. 14, 2006
(86) PCT No.: PCT/EP2006/060687

§ 371 Date: Sep. 7, 2007

(30) Foreign Application Priority Data
Mar. 15, 2005 (IT) FI2005A000041
Nov. 21, 2005 (IT) FI2005A000239

(51) Int. Cl. A61K 31/445; C07D 211/32

(52) U.S. Cl. 514/331; 546/233

(57) ABSTRACT

Compounds of structure (I) having histone deacetylase (HDAC) inhibitor activity are described. The compounds are chemically characterised by the presence of hydroxamic acid, with zinc as chelator, and an aromatic nucleus connected by a linker in which either a bicyclic aromatic system or a piperidino ring are present.

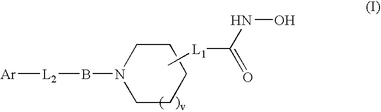

### FIELD OF THE INVENTION

The present invention relates to histone deacetylase inhibitor compounds characterised by the presence of a hydroxamic group, to preparations for obtaining them and to their use for preparing pharmaceutical formulations to be employed for treating pathologies in which the mechanism of gene regulation plays an essential role.

In particular the present invention concerns a compound having a general formula (I):

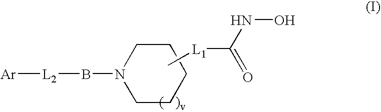

in which v=0, 1, 2
B is a bond or is chosen from the group: —O—, —NR5-, —CO—, —NR5-CO—, —O—CO—, —SO₂—, —NR5-SO₂—, or represents one of the following structures:

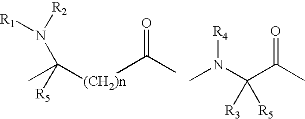

in which n=0, 1, 2
in which R5 is a H or a C1-3 alkyl
R1 represents a H or is chosen from the group: C1-3 alkyl, C1-3 acyl or an acyl derived from one of the following acids: benzoic, phenylacetic, benzothiophene-carboxylic, indole-carboxylic
R2 represents a H or a C1-3 alkyl group
   or R1 and R2 represent jointly with the nitrogen atom, a five- or six-membered heterocycle chosen from pyrrolidine, piperidine, piperazine, morpholine, thiomorpholine
R3 represents H or is chosen from the group: —C1-6 alkyl, —C1-6 alkylene-W, where W is chosen from —OR5, —SR5, —CONR7R8, —NR7R8, —OCOR6-NR5COR6, guanidine, and R7 and R8 independently represent a H or C1-3 alkyl group or R7 and R8 represent jointly with the nitrogen atom a five- or six-membered heterocycle chosen from pyrrolidine, piperidine, piperazine, morpholine, thiomorpholine, and R6 is chosen from the group: hydrogen, C1-3 alkyl, Ar1 where Ar1 is an aromatic group chosen from phenyl, naphthyl, pyridine, quinoline, indole, benzofuran, benzothiophene and can possibly be substituted with up to three groups independently chosen from: C1-3 alkyl, OR9, SR9, NR9R10, N(R9)COR10, NO₂, CN, F, Cl, Br, —CF₃, COOR9, CONR9R10, CH₂NR9R10, N(R9)SO₂R10, CH₂OR9, SOH, CH₂SO₃H, in which R9 is a group chosen from H, C1-3 alkyl, —(CH₂)q-NR10R11, pyrrolidine, R10 and R11 are independently a group chosen from H and C1-3 alkyl, q can assume the values 2 or 3
R4 is a group chosen from H and C1-3 alkyl
   or R4 and R3 represent jointly with the nitrogen atom a heterocycle chosen from pyrrolidine or piperidine
L1 indicates a group attached to any of the carbon atoms in the heterocyclic ring and is chosen from the group: alkylidene of the type —(CH₂)m-, possibly substituted on each C with one or two methyl groups, and in which m can assume the values 1, 2, 3, 4, 5, or an alkene chosen from —(CH₂)e-CH═CH—(CH₂)f-, —(CH₂)g-CH═CH—(CH₂)h-CH═CH—(CH₂)l- possibly substituted on each C with one or two methyl groups and in which e, f, g, h and l can independently assume the values 0, 1, 2, 3 or 4
L2 indicates a bond or a group chosen from: —(CH₂)p-, —(CH₂)p-CH═CH—, —(CH₂)p-T-(CH₂)z-, —(CH₂)p-CO—, —(CH₂)p-CH═CH—CO—, —CO-T-(CH₂)z-, —(CH₂)p-T-CO—, each group being possibly substituted on the Cs with one or two methyl groups and in which p and z can independently assume the values 0, 1, 2, 3 or 4 and T is chosen from —O—, —S—, —NR5-
Ar represents a group derived from the following aromatic systems: phenyl, pyridyl, furyl, pyrimidyl, pyrazyl, piperazyl, triazolyl, tetrazolyl, biphenyl, imidazolyl, naphthyl, quinoline, isoquinoline, diphenyl-methyl, benzofuryl, dihydrobenzofuryl, benzothienyl, indolyl, benzothiazolyl, benzoxazolyl, benzoisoxazolyl, oxazolyl-phenyl, thiodiazolyl-phenyl, pyridyl-phenyl, pyrazolyl-phenyl, thiazolyl-phenyl, furyl-phenyl, thienyl-phenyl, benzyloxy-phenyl, tetrazolyl-phenyl, phenyl-oxazolyl, phenyl-pyrazolyl-, phenyl-thiazolyl, phenyl-thiadiazolyl, phenyl-isothiazolyl, phenyl-oxadiazolyl, phenyl-isoxazolyl, phenyl-imidazolyl, phenyl-triazolyl, phenyl-furyl, phenyl-thiophenyl, phenyl-pyrrolyl, phenyl-pyrrolidyl, indanyl, fluorenyl, benzopyranyl, dihydrobenzopyranyl, benzodioxolyl, phenoxy-phenyl, benzoxazinyl, dihydrobenzoxazinyl in which each group can possibly be substituted with up to three groups independently chosen from: C1-3 alkyl, OR9, SR9, NR9R10, N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, CH₂SO₃H, in which R9 is a group chosen from H, C1-4 alkyl, —(CH₂)q-NR10R11, pyrrolidine, R10 and R11 are independently chosen from H and C1-3 alkyl, q can assume the values 1, 2 or 3,
or Ar-L2-B can jointly be chosen from the group consisting of:

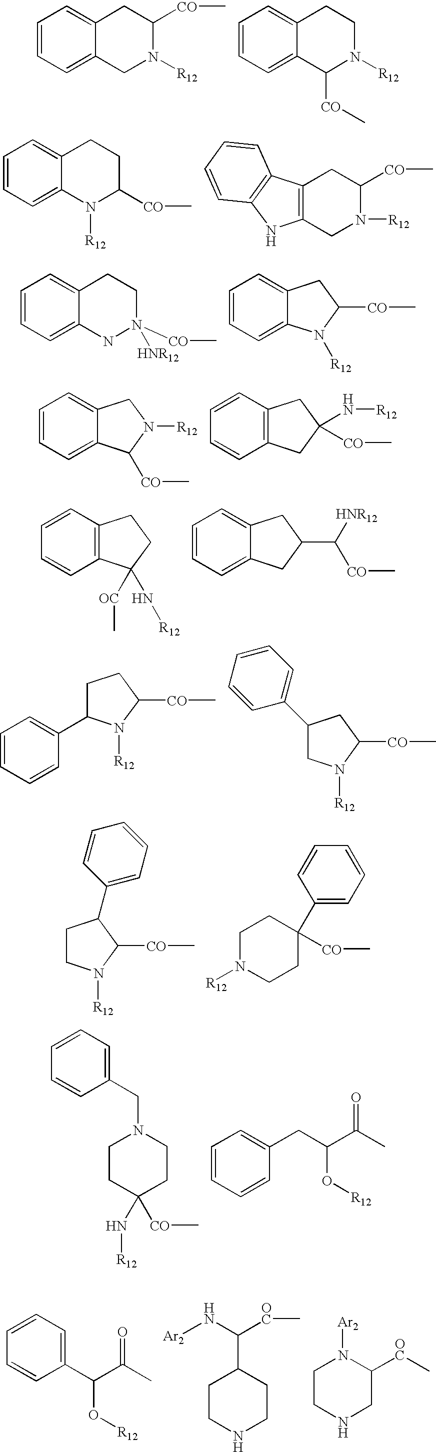

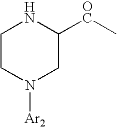

in which:
the R12 group represents a H, a C1-3 alkyl or a C1-3 acyl
the Ar2 group is an aromatic chosen from benzyl or an acyl group derived from benzoic, phenylacetic or benzothiophene-carboxylic acids
the aromatic part can be substituted with up to three groups independently chosen from: C1-3 alkyl, OR9, SR9, NR9R10, N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, CH₂SO₃H, in which R9 is a group chosen from H, C1-4 alkyl, —(CH₂)q-NR10R11, pyrrolidine, R10 and R11 are independently a group chosen from H and C1-3 alkyl, q can assume the values 1, 2 or 3.

To be considered excluded from the present invention are products in which:

B is chosen from —NR5-CO— or —NR5-SO— and at the same time

L2 is chosen from —(CH₂)p-CO— or —(CH₂)p-CH═CH—CO—. The relative prodrugs of general formula (III) and (IV) also form part of the present invention

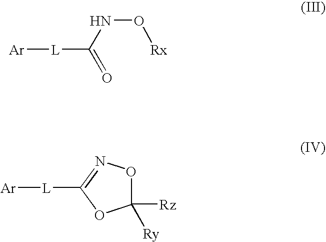

In which Rx can be Ac, COEt, CO-nPr, CO-iPr, CO-tBu, benzoyl, pNO₂benzoyl, CH₃, Et, nPr, iPr, tBu, Benzyl, tetrahydropyranyl.

Ry and Rz independently indicate a H or a C1-3 alkyl group.

Also forming part of the present invention are all possible optical isomers, such as enantiomers and/or diastereoisomers, derived from the possible presence of chiral centres in the compounds of general formula (I), and possible mixtures thereof either as racemes or in various ratios thereof, and their inorganic and organic acid salts.

### STATE OF THE ART

Histone deacetylase is known to have an essential role in the mechanism that regulates gene expression. Inhibitors of histone deacetylase (HDAC) induce hyperacetylation of histones, with consequent alteration of gene expression itself. It follows that said inhibitors are useful as therapeutic or prophylactic agents for pathological states caused by abnormal gene expression, such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like.

The enzyme histone deacetylase is already well known and, via X-ray and SAR studies of various inhibitor classes, the structural characteristics that a potential inhibitor should possess have been elucidated; in particular a) a domain able to bind a metal (specifically Zn), b) a linker able to occupy a channel of the enzyme, c) a surface recognition domain that interacts with the structures on the rim of the enzyme active site (J. Med. Chem., 2003, 46(24), 5097-5116).

In the last few years many examples of HDAC inhibitors with the aforesaid structural characteristics have become apparent.

For example, compounds that present a n-hydroxyamide and a linear linker are described in: Bioorganic & Medicinal Chem Letters (2002), 12, 2919-2923; J Med Chem (2002) 45 (13), 2877-2885; J Med Chem (2002), 45 (4), 753-757; Bioorganic & Medicinal Chem Letters (2004), 14, 449-453. Other publications demonstrate hydroxamic acids in which the linker is not linear; in Bioorganic & Medicinal Chem Letters (2001), 11, 2847-2890 the linker is represented by phenyl-ethyl or styryl; in Bioorganic & Medicinal Chem Letters (2002), 12, 1347-1349 the linker is a phenyl or a cyclohexyl; the compounds described in WO2004013130 present a linker consisting of athiophene.

Other authors have shown the possibility of substituting hydroxamic acid with other groups able to bind the metal of the enzyme active site, for example with amides (J. Med Chem (2003), 46, 820-830; or in EP847992) or electrophile ketones. WO2004069133 describes compounds in which, based on the aforementioned scheme, the metal binding group is represented by a phenylenediamine amide, and the linker by a heterocycle chosen from indole, benzothiophene or benzofuran. WO2005040101 also claims hydroxamates containing carbamoyl piperidino groups or the like as general meanings.

Notwithstanding all that is already known on the subject, there is still however a great need to identify new HDAC inhibitors which allow us to prepare new drugs for the treatment of the many pathologies that are potentially curable via this mechanism of action.

### DETAILED DESCRIPTION OF THE INVENTION

The aim of the present invention is to provide new HDAC inhibitors of general formula (I), useful as drugs, and the pharmaceutical compositions that contain them, as active ingredients for the treatment or prophylaxis of pathologies such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like.

The present invention provides compounds of general formula (I) as heretofore described.

A group of preferred compounds of the present invention are those of general formula (II)

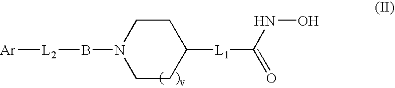

in which v=1
B is a bond, or is chosen from the group: —CO—, —NR5-CO—, —O—CO—, —SO₂—, —NR5-SO₂—, or represents one of the following structures:

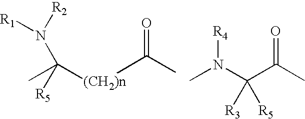

in which n=0, 1, 2
in which R5 is a H or a C1-3 alkyl
R1 represents a H or is chosen from the group: C1-3 alkyl, C1-3 acyl or an acyl derived from one of the following acids: benzoic, phenylacetic, benzothiophene-carboxylic, indole-carboxylic
R2 represents a H or a C1-3 alkyl group
   or R1 and R2 represent jointly with the nitrogen atom, a five- or six-membered heterocycle chosen from pyrrolidine, piperidine, piperazine, morpholine, thiomorpholine
R3 represents H or is chosen from the group: —C1-6 alkyl, —C1-6 alkylene-W, where W is chosen from —OR5, —SR5, —CONR7R8, —NR7R8, —OCOR6-NR5COR6, guanidine, and R7 and R8 independently represent a H or a C1-3 alkyl group or R7 and R8 represent jointly with the nitrogen atom a five- or six-membered heterocycle chosen from pyrrolidine, piperidine, piperazine, morpholine, thiomorpholine, and R6 is chosen from the group hydrogen, C1-3 alkyl, Ar1 where Ar1 is an aromatic group chosen from phenyl, naphthyl, pyridine, quinoline, indole, benzofuran, benzothiophene and can possibly be substituted with up to three groups independently chosen from: C1-3 alkyl, OR9, SR9, NR9R10, N(R9)COR10, NO₂, CN, F, Cl, Br, —CF₃, COOR9, CONR9R10, CH₂NR9R10, N(R9)SO₂R10, CH₂OR9, SOH, CH₂SO₃H, in which R9 is a group chosen from H, C1-3 alkyl, —(CH₂)q-NR10R11, pyrrolidine, R10 and R11 are independently a group chosen from H and C1-3 alkyl, q can assume the values 2 or 3
R4 represents an H or a C1-3 alkyl group
   or R4 and R3 represent jointly with the nitrogen atom a heterocycle chosen from pyrrolidine or piperidine
L1 is chosen from the group: alkylidene of the type —(CH₂)m-, possibly substituted on each C with one or two methyl groups, and in which m can assume the values 1, 2, 3
L2 indicates a bond or a group chosen from: —(CH₂)p-, —(CH₂)p-CH═CH—, —(CH₂)p-T-(CH₂)z-, —(CH₂)p-CO—, —(CH₂)p-CH═CH—CO—, —CO-T-(CH₂)z-, —(CH₂)p-T-CO—, each group being possibly substituted on the Cs with one or two methyl groups and in which p and z can independently assume the values 0, 1, 2, 3 or 4 and T is chosen from —O—, —S—, —NR5-
Ar represents a group derived from the following aromatic systems: phenyl, pyridyl, furyl, pyrimidyl, pyrazyl, piperazyl, triazolyl, tetrazolyl, biphenyl, imidazolyl, naphthyl, quinoline, isoquinoline, diphenyl-methyl, benzofuryl, dihydrobenzofuryl, benzothienyl, indolyl, benzothiazolyl, benzoxazolyl, benzoisoxazolyl, oxazolyl-phenyl, thiodiazolyl-phenyl, pyridyl-phenyl, pyrazolyl-phenyl, thiazolyl-phenyl, furyl-phenyl, thienyl-phenyl, benzyloxy-phenyl, tetrazolyl-phenyl, phenyl-oxazolyl, phenyl-pyrazolyl-, phenyl-thiazolyl, phenyl-thiadiazolyl, phenyl-isothiazolyl, phenyl-oxadiazolyl, phenyl-isoxazolyl, phenyl-imidazolyl, phenyl-triazolyl, phenyl-furyl, phenyl-thiophenyl, phenyl-pyrrolyl, phenyl-pyrrolidyl, indanyl, fluorenyl, benzopyranyl, dihydrobenzopyranyl, benzodioxolyl, phenoxy-phenyl, benzoxazinyl, dihydrobenzoxazinyl in which each group can possibly be substituted with up to three groups independently chosen from: C1-3 alkyl, OR9, SR9, NR9R10, N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, CH₂SO₃H, in which R9 is a group chosen from H, C1-4 alkyl, —(CH₂)q-NR10R11, pyrrolidine, R10 and 11 are independently chosen from H and C1-3 alkyl, q can assume the values 1, 2 or 3,
or Ar-L2-B can jointly be chosen from the group consisting of:

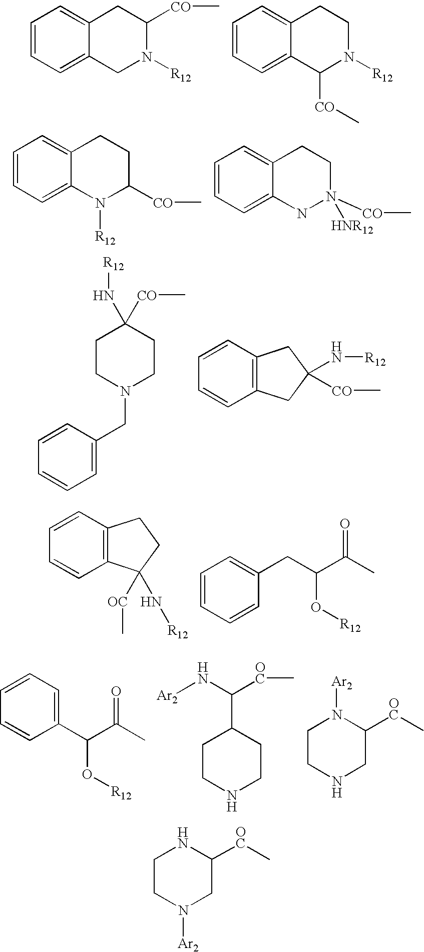

in which:
the R12 group represents a H, a C1-3 alkyl or a C1-3 acyl
the Ar2 group is an aromatic chosen from benzyl or an acyl group derived from benzoic, phenylacetic or benzothiophene-carboxylic acids
the aromatic part can be substituted with up to three groups independently chosen from: C1-3 alkyl, OR9, SR9, NR9R10, N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, CH₂SO₃H, in which R9 is a group chosen from H, C1-4 alkyl, —(CH₂)q-NR10R11, pyrrolidine, R10 and R11 are independently a group chosen from H and C1-3 alkyl, q can assume the values 1, 2 or 3.

To be considered excluded from the present invention are products in which:

B is chosen from —NR5-CO— or —NR5-SO₂— and at the same time
L2 is chosen from —(CH₂)p-CO— or —(CH₂)p-CH═CH—CO—.

The relative prodrugs of general formula (III) and (IV) also form part of the present invention

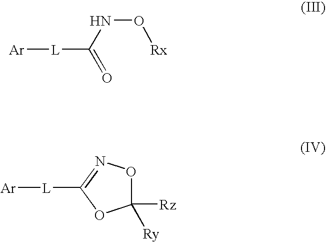

in which Rx can be Ac, COEt, CO-nPr, CO-iPr, CO-tBu, Benzoyl, pNO₂-Benzoyl, CH₃, Et, nPr, iPr, tBu, Benzyl, tetrahydropyranyl
Ry and Rz independently indicate a H or C1-3 alkyl group.

Also forming part of the present invention are all possible optical isomers, such as enantiomers and/or diastereoisomers, derived from the possible presence of chiral centres in the compounds of general formula (I), and possible mixtures thereof either as racemes or in various ratios thereof, and their inorganic and organic acid salts.

A group of particularly preferred compounds of the present invention are those of general formula (II) in which v=1
B is a bond, or is chosen from the group: —CO—, —NR5-CO—, —O—CO—, or represents one of the following structures:

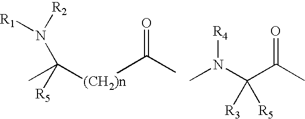

in which n=0, 1
in which R5 is a H or a C1-3 alkyl,
R1 represents a H or is chosen from the group: C1-3 alkyl, C1-3 acyl
R2 represents a H or a C1-3 alkyl group
R3 represents H or is a —C1-6 alkylene-W, where W is chosen from —OR5, —NR7R8 and R7 and R8 independently represent a H or C1-3 alkyl group
R4 represents a H or a C1-3 alkyl group,
L1 is chosen from the group: alkylidene of the type —(CH₂)m-, possibly substituted on each C with one or two methyl groups, and in which m can assume the values 2, 3
L2 indicates a bond or a group chosen from: —(CH₂)p-, —(CH₂)p-CH═CH—, —(CH₂)p-T-(CH₂)z-, —(CH₂)p-CO—, —CO-T-(CH₂)z-, —(CH₂)p-T-CO—, each group being possibly substituted on the Cs with one or two methyl groups and in which p and z can independently assume the values 0, 1, 2, 3, and T is chosen from: —O—, —S—, —NR5-
Ar represents a group derived from the following aromatic systems: phenyl, pyridyl, furyl, triazolyl, biphenyl, naphthyl, quinoline, isoquinoline, benzofuryl, benzothienyl, indolyl, benzothiazolyl, benzoisoxazolyl, oxazolyl-phenyl, thiodiazolyl-phenyl, pyridyl-phenyl, pyrazolyl-phenyl, thiazolyl-phenyl, furyl-phenyl, thienyl-phenyl, benzyloxy-phenyl, tetrazolyl-phenyl, phenyl-oxazolyl, phenyl-pyrazolyl-, phenyl-thiazolyl, phenyl-thiadiazolyl, phenyl-isothiazolyl, indanyl, fluorenyl, benzodioxolyl, phenoxy-phenyl, in which each group can possibly be substituted with up to two groups independently chosen from C1-3 alkyl, OR9, NR9R10, N(R9)COR10, CN, F, Cl, Br, —CF₃, —SCF₃, —(CH₂)q-NR9R10, in which R9 is a group chosen from H, C1-4 alkyl, R10 and 11 are independently a group chosen from H and C1-3 alkyl, q can assume the values 2 or 3, or Ar-L2-B can jointly be chosen from the group consisting of

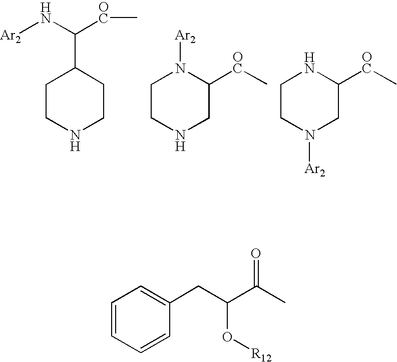

in which:
the R12 group represents a H, a C1-3 alkyl
the Ar2 group is an aromatic chosen from benzyl or an acyl group derived from benzoic, phenylacetic or benzothiophene-carboxylic acids
the aromatic part can be substituted with up to three groups independently chosen from: a C1-3 alkyl, OR9, NR9R10, N(R9)COR10, CN, F, Cl, Br, —CF₃, —SCF₃, —(CH₂)q-NR9R10, in which R9 is a group chosen from H, C1-4 alkyl, R10 and R11 are independently a group chosen from H and C1-3 alkyl, q can assume the values 2 or 3. To be considered excluded from the present invention are products in which:
   B is chosen from —NR5-CO— and at the same time L2 is chosen from —(CH₂)p-CO—. In the present invention the following meanings are preferred: for C1-3 alkyl a group chosen from methyl, ethyl, propyl, isopropyl, for C1-4 alkyl a group chosen from methyl, ethyl, propyl, isopropyl, butyl, isobutyl, tert-butyl, for C1-6 alkyl a group chosen from methyl, ethyl, propyl, isopropyl, butyl, isobutyl, tert-butyl, pentyl, hexyl, for C1-6 alkylidene a group chosen from methylene, ethylene, propylene, methylethylene, tetramethylene, methylpropylene, pentamethylene, hexamethylene, for C1-3 acyl a group chosen from formyl, acetyl, propionyl.

Furthermore, possibly preferred salts are intended as salts obtained with inorganic or organic acids chosen from: hydrochloric, hydrobromic, phosphoric, sulphuric, nitric, acetic, trifluoroacetic, methanesulfonic, toluenesulfonic, oxalic, succinic, malonic, adipic, benzoic acids.

The HDAC inhibitors of the present invention can be synthesised according to reactions known in the state of the art, but which can vary greatly on the basis of the series of synthesis steps needed to prepare the individual compounds summarized in general formula (I).

Some descriptive schemes by way of non-limiting examples are given below. From said schemes the synthesis of all the compounds represented in general formula (I) can be easily deduced or extrapolated by experts of the art:

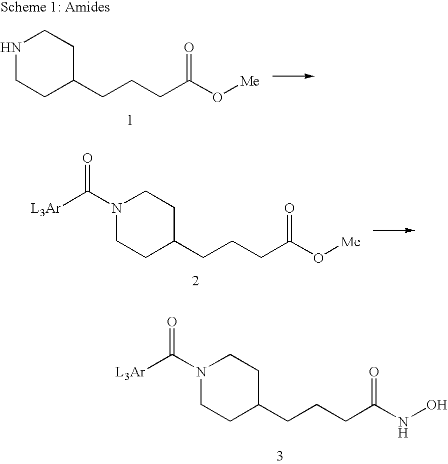

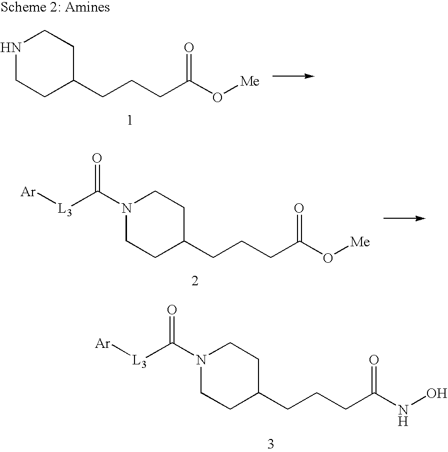

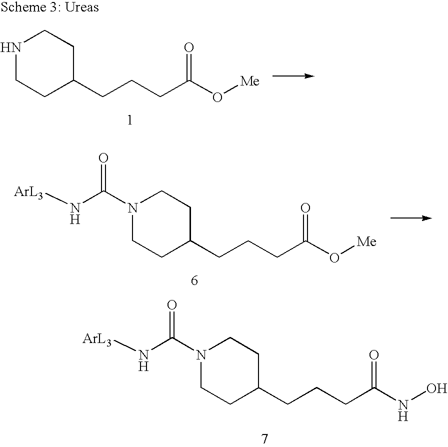

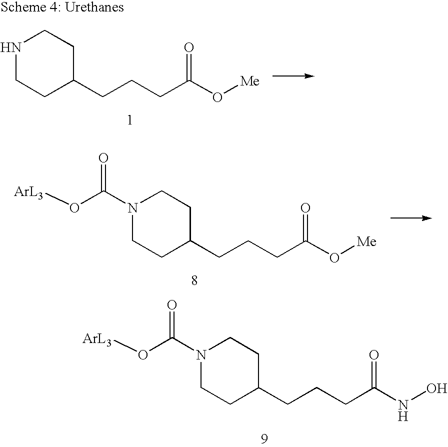

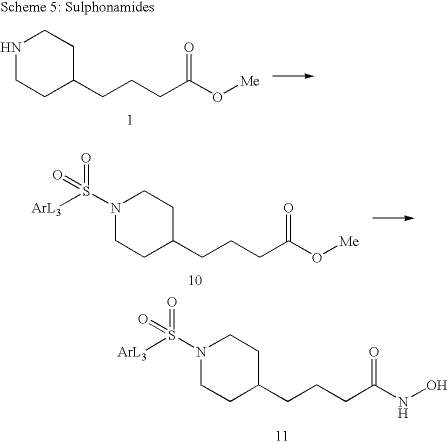

In some cases the formation of amide bonds also allows passage along the solid phase synthesis paths developed in peptide chemistry based on the use of resin. Some representative examples of the present invention and the method for their synthesis are given below.

### General Procedures

### Synthesis of Type 2 Amides

### 4-(1-phenylacetyl-piperidin-4-yl)-butyric acid methyl ester

DIPEA (0.11 ml, 0.66 mmols) and phenylacetyl chloride (0.043 ml, 0.33 mmols) are added to a suspension of 4-piperidino butyric acid methyl ester hydrochloride (100 mg, 0.33 mmols) in anhydrous CH₂Cl₂ (10 ml), maintained at 0° C. under N₂. The resulting mixture is left under agitation for 2 hours at ambient temperature.

At the end of the reaction (HPLC monitoring) the mixture is diluted with CH₂Cl₂ and 15 ml of 5% NaHCO₃ are added. The two phases are separated and the organic phase is then further washed with 5% NaHCO₃ (3 times) and ssNaCl. The organic phase is dried over Na₂SO₄ and the solvent evaporated under reduced pressure to obtain the desired amide with quantitative yield.

Synthesis of the hydroxaminic acids 3, 5, 7, 9, 11 starting from the corresponding esters (method A) or carboxylic acids (method B).

### Method A:

A solution of KOH (210 mg, 3.74 mmols) in MeOH (2 mL) is added to a solution of hydroxylamine hydrochloride (190 mg, 2.72 mmols) in MeOH (2 mL) maintaining the temperature at 0° C. The solution is then stirred for 15 minutes while at 0° C. A solution of the methyl ester (0.34 mmols) in MeOH/THF (4 mL) is added and the resulting mixture maintained at ambient temperature for 12 hours under agitation. At the end of the reaction (HPLC monitoring) the pH is adjusted to about 8 by adding a 6N HCl solution.

At this point extraction in the organic phase can be undertaken if the product is not too water-soluble, or, otherwise, purification by inverse phase chromatography.

### Method B:

### N-Hydroxy-4-(1-phenylacetyl-piperidin-4-yl)-butyramide

DIPEA (0.085 ml, 0.48 mmols) and BSA (0.234 ml, 0.96 mmols) are added to a solution of 4-piperidine butyric acid hydrochloride (100 ml, 0.48 mmols) in anhydrous CH₂Cl₂ (10 ml). The mixture is allowed to react at ambient temperature for 2 hours, then phenylacetyl chloride (0.063 ml, 0.48 mmols) is added. The resulting mixture is stirred at ambient temperature for 12 hours.

At the end of the reaction (HPLC monitoring) 15 ml of 5% NaHCO₃ are added and the mixture is left under vigorous agitation for 30 minutes. The two phases are separated and the aqueous phase is acidifed to pH 2.

The aqueous phase is extracted with EtOAc, the organic extracts are washed with ssNaCl, dried over Na₂SO₄ and the solvent evaporated under reduced pressure to obtain the desired carboxylic acid (130 mg, 95%) with suitable purity.

COCl₂ (0.045 ml, 0.54 mmols) and a catalytic quantity of DMF are added to a solution of the acid (130 mg, 0.45 mmols) in 5 ml of anhydrous CH₂Cl₂. After two hours the CH₂Cl₂ is evaporated under reduced pressure, twice redissolving and re-evaporating the crude reaction product again using CH₂Cl₂. To the acyl chloride thus formed, dissolved in 5 ml of anhydrous CH₂Cl₂, are slowly added 5 ml of an aqueous solution of NH₂OH HCl (32 mg, 0.45 mmols) and NaHCO (75 mg, 0.9 mmols). The mixture is allowed to react for 1 hour under vigorous agitation at ambient temperature.

At the end of the reaction (HPLC monitoring) the two phases are separated, the organic phase is washed with ssNaCl, dried over Na₂SO₄ and concentrated under reduced pressure. The crude product obtained is purified by phase inverse chromatography, to obtain 82 mg of the desired product (60%).

### Synthesis of the Amines 4:

### 4-(1-Phenethyl-piperidin-4-yl)-butyric acid methyl ester

Phenylacetic aldehyde (0.046 mL, 0.40 mmols) and sodium triacetoxyborohydride (190 mg, 0.90 mmols) are added sequentially to a solution of 4-piperidin-4-yl-butyric acid methyl ester hydrochloride salt (80 mg, 0.36 mmols) in THF (3 mL). The resulting mixture is stirred at ambient temperature for four hours. 10% NaHCO (10 mL) and EtOAc (10 mL) are then added and the two phases separated. The organic phase is washed with water, ssNaCl and dried over Na₂SO₄. Filtration and removal of solvents by reduced pressure provide the desired product with a quantitative yield, which can be used as such in the following reaction.

### Synthesis of the Ureas 6:

### 4-(1-Benzylcarbamoyl-piperidin-4-yl)-butyric acid methyl ester

A solution of 4-piperidin-4-yl-butyric acid methyl ester hydrochloride salt (98 mg, 0.443 mmols), DIPEA (0.091 mL, 0.532 mmols) and benzylisocyanate (0.051 mL, 0.499 mmols) in dichloromethane (4 mL) is stirred at ambient temperature for 3 hours.

At the end of the reaction (HPLC monitoring) a portion of aminomethyl polystyrene resin (50 mg, 1 mmol/g loading) is added to remove excess isocyanate and the resulting mixture is stirred for a further 2 hours. The solution is filtered and concentrated under reduced pressure to obtain the desired product (140 mg, 99%) which can be used as such in the following reaction.

### Synthesis of the Sulfonamides 10:

### 4-(1-Benzenesulfonyl-piperidin-4-yl)-butyric acid methyl ester

A portion of benzenesulfonyl chloride (45 mg, 0.26 mmols) and one of DIPEA (70 mg, 0.54 momls) are added to a solution of 4-piperidin-4-yl-butyric acid methyl ester hydrochloride salt (60 mg, 0.27 mmols) in anhydrous CH₂Cl₂ (2 mL).

At the end of the reaction (HPLC monitoring) the mixture is diluted with CH₂Cl₂ and washed with 5% NaHSO₄, 5% NaHCO₃ and ssNaCl; the organic phase is dried over NaSO₄ and evaporated. The crude product thus obtained is purified by chromatography (silica, petroleum ether/AcOt 8:2). 75 mg (90%) of the desired product are obtained with suitable purity.

### Example of Synthesis of Hydroxamine Acids of Type 3, 7, 9, 11 in the Solid Phase

### 4-[1-(2-(R)-Amino-3-phenyl-propionyl)-piperidin-4-yl]-N-hydroxy-butyramide

200 mg of the commercial resin N-Fmoc-hydroxylamine-2-chlorotrityl polystyrene (conc. 0.5 mmols/g) is allowed to pre-swell for 1 hour in 2 ml of dichloromethane (DCM). The resin is filtered off and suspended in 2 ml of a solution of 20% piperidine in dichloromethane. After 30 minutes the resin is filtered off and resuspended in 2 ml of a solution containing 20% piperidine in DMF. After 30 minutes the resin is filtered off and washed with DCM (4×2 ml). At the end the resin is re-swollen in DCM for 20 minutes.

A solution of HOAt (68 mg, 0.50 mmols) in 1.5 ml of anhydrous DMF is added to a solution of 4-[N-(fluoren-9-ylmethoxycarbonyl)piperidin-4-yl]-butanoic acid (200 mg, 0.50 mmols) in 1.5 ml of DCM. 0.08 ml of diisopropylcarbodiimide (63 mg, 0.50 mmols) are slowly added and the mixture is maintained for 1 hour under agitation at ambient temperature. The solution is poured onto polystyrene resin and the suspension stirred for 18 hours at ambient temperature. The resin is filtered off and suspended in 2 ml of a solution of 20% piperidine in dichloromethane. After 30 minutes the resin is filtered off and washed sequentially with DMF (2×2 ml) and DCM (2×2 ml). At the end the resin is re-swollen in DCM for 20 minutes.

The qualitative ninhydrin assay on completion of the acylation reaction (Kaiser et al Anal. Biochem. 1970, 34, 595) proves to be negative.

A solution of HOAt (68 mg, 0.05 mmols) in 1.5 ml anhydrous DMF is added to a solution of Fmoc-D-Phe-OH (194 mg, 0.50 mmols) in 1.5 ml of DCM. Diisopropylcarbodiimide (0.08 ml, 63 mg, 0.50 mmols) is slowly added and the mixture maintained for 1 hour at ambient temperature under agitation. The solution is poured onto the polystyrene resin and the suspension agitated for 18 hours at ambient temperature. The resin is filtered off and suspended in 2 ml of a solution of 20% piperidine in dichloromethane. After 30 minutes the resin is filtered off and washed sequentially with DMF (2×2 ml) and DCM (2×2 ml). 100 μl of trifluoroacetic acid and 100 μl of triethylsilane are added to the thus obtained resin, re-swollen in 2 ml DCM for 20 minutes. It is maintained under agitation for 15 minutes at ambient temperature. The resin is filtered off and washed twice with DCM and MeOH alternately. The filtrates are pooled and evaporated to dryness. Grinding the crude product thus obtained with ethyl ether provides the desired product (35 mg, 80% yield) with a purity of 95%. MF:C₁₈H₂₇N₃O₃, MW: 333.43.

### 4-[1-(2-(R)-Acetylamino-3-phenyl-propionyl)-piperidin-4-yl]-N-hydroxy-butyramide

0.048 ml (51 mg, 0.50 mmols) of Ac₂O and 0.085 ml of DIPEA (65 mg, 0.50 mmols) in DCM are added to the resin obtained in step F, re-swollen in 2 ml of DCM for 20 minutes. The resin is agitated for 18 hours at ambient temperature then filtered off and washed sequentially with DMF (2×2 ml) and DCM (2×2 ml). 100 μl of trifluoroacetic acid and 100 μl of triethylsilane are added to the thus obtained resin, re-swollen in 2 ml DCM for 20 minutes. It is maintained under agitation for 15 minutes at ambient temperature. The resin is filtered off and washed twice with DCM and MeOH alternately. The filtrates are pooled and evaporated to dryness. Grinding the crude product thus obtained with ethyl ether provides the desired product (28 mg, 75% yield) with a purity of 95%. MF: C₂₀H₂₉N₃O₄, MW: 375.47.

### Benzo[b]thiophene-2-carboxylic acid 2-[(benzo[b]thiophene-2-carbonyl)-(R)-amino]-3-[4-(3-hydroxycarbamoyl-propyl)-piperidin-1-yl]-3-oxo-propyl ester

A mixture of HOAt (20 mg, 0.15 mmols), Fmoc-D-Ser-OH (49 mg, 0.15 mmols) and diisopropylcarbodiimide (0.024 ml, 18 mg, 0.15 mmols) in 3 ml of DMF/DCM, previously maintained at ambient temperature for an hour, is added to the resin obtained in Step C, re-swollen in 2 ml of DCM for 20 minutes. The suspension is agitated for 18 hours at ambient temperature then the resin is filtered off and washed sequentially with DMF (2×2 ml) and DCM (2×2 ml).

The resin is filtered off and suspended in 2 ml of a solution of 20% piperidine in dichloromethane. After 30 minutes the resin is filtered off and washed sequentially with DMF (2×2 ml) and DCM (2×2 ml).

To the resin obtained in Step E and re-swollen in 2 ml of DCM for 20 minutes are added the mixture of HOAt (68 mg, 0.5 mmols), benzo[b]thiophene-2-carboxylic acid (90 mg, 0.5 mmols) and 0.08 ml of diisopropylcarbodiimide (63 mg, 0.5 mmols) in 3 ml of DMF/DCM previously held at ambient temperature for 1 hour. The suspension is agitated for 18 hours at ambient temperature. The resin is filtered off and washed sequentially with DMF (2×2 ml) and DCM (2×2 ml).

100 μl of trifluoroacetic acid and 100 μl of triethylsilane are added to the thus obtained resin, re-swollen in 2 ml DCM for 20 minutes. It is maintained under agitation for 15 minutes at ambient temperature. The resin is filtered off and washed twice with DCM and MeOH alternately. The filtrates are pooled and evaporated to dryness. Grinding the crude product thus obtained with ethyl ether provides the desired product (41 mg, 75% yield) with a purity of 95% (HPLC, 214 nm). MF: C₃₀H₃₁N₃O₆S₂, MW: 593.73.

The relative prodrugs of general formula (IV) and (V) also form part of the present invention, for whose production the preparation of suitable syntones in which the hydroxamic acid function is protected in the hydrolysable group of the ‘prodrug’ is generally preferred.

These syntones are then used for synthesis of the required compounds by synthetic paths totally similar to those used for compounds not defined as ‘prodrugs’.

Non-limitative examples of synthesis of syntones for prodrugs are:

### Synthesis of Prodrug A:

### 4-(3-Acetoxycarbamoyl-propyl)-piperidine-1-carboxylic acid benzylamide

Pyridine (0.019 ml, 0.23 mmols, 1.5 eq.) and acetic anhydride (0.022 ml, 0.23 mmols, 1.5 eq.) are added to a solution of 4-(3-hydroxycarbamoyl-propyl)-piperidine-1-carboxylic acid benzylamide (50 mg, 0.16 mmols, 1.0 eq.) in anhydrous DCM (3 ml) and the resulting mixture allowed to react at ambient temperature for 12 hours. At the end of the reaction, after diluting with DCM and washing with 1 N HCl (5 ml), the phases are separated. The organic phase is washed washing with ssNaCl, dried over Na₂SO₄ and concentrated under reduced pressure. The crude product obtained is purified by flash chromatography (30% petroleum ether in AcOEt then AcOEt) to obtain 45 mg of the desired monoacetylated product as a white solid (80%).

¹H NMR (DMSO-d₆, 400 MHz): δ (ppm) 0.90-1.03 (2H, m), 1.16-1.29 (2H, m), 1.32-1.48 (1H, m), 1.48-1.67 (4H, m), 2.11 (2H, t, J=6.8 Hz, CH₂CO), 2.15 (3H, s, OCH₃), 2.65 (2H, m, N(CHH)₂), 3.98 (2H, m, N(CHH)₂), 4.24 (2H, d, J=5.2 Hz, PhCH₂), 6.97 (1H, m, NCONH), 7.16-7.36 (5H, m, ArH), 11.53 (1H, s, NHO)

MF: C₁₉H₂₇N₃O₄, MW: 361.43

### Synthesis of Prodrug B

### [3-(5,5-Dimethyl-[1,4,2]dioxazol-3-yl)-propyl]-piperidine-1-carboxylic acid benzylamide

Camphorsulfonic acid (37 mg, 0.16 mmols, 1.0 eq.) is added to a solution of 4-(3-hydroxycarbamoyl-propyl)-piperidine-1-carboxylic acid benzylamide (50 mg, 0.16 mmols, 1.0 eq.) and 2,2-diethoxypropane (0.076 mL, 0.47 mmols, 3.0 eq.) in anhydrous DCM (3 ml) and the resulting mixture is left under agitation at ambient temperature for 4 hours. At the end of the reaction a saturated aqueous solution of Na₂CO₃ (5 ml) is added and the phases separated. The aqueous phase is washed with Et₂O (4×5 ml). The pooled organic phase is finally dried over Na₂SO₄ and concentrated under reduced pressure. The crude product is purified by flash chromatography (30% petroleum ether in AcOEt) to obtain 45 mg of the desired product (80%).

¹H NMR (DMSO-d₆, 400 MHz): 6 (ppm) 0.87-0.96 (2H, m), 1.17-1.30 (2H, m), 1.30-1.45 (1H, m), 1.46-1.68 (4H, m), 1.49 (6H, s, 2×CH₃), 2.28 (2H, t, J=7.2 Hz, CH₂CO), 2.63 (2H, m, N(CHH)₂), 3.96 (2H, m, N(CHH)₂), 4.22 (2H, d, J=5.2 Hz, PhCH₂), 7.01 (1H, m, NCONH), 7.16-7.33 (5H, m, ArH)

MF: C₂₀H₂₉N₃O₃, MW: 359.46.

### Synthesis of Prodrug C:

### 4-[3-(Tetrahydro-pyran-2-yloxycarbamoyl)-propyl]-piperidine-1-carboxylic acid benzylamide

EDAC (69 mg, 0.36 mmols, 1.0 eq.) and HOBt (49 mg, 0.36 mmols, 1.0 eq) are added to a solution of 4-(1-benzylcarbamoyl-piperidin-4-yl)-butyric acid (100 mg, 0.328 mmols, 1.0 eq.) in anhydrous DMF (5 mL) and the mixture maintained under agitation at ambient temperature for 1 hour. O-(tetrahydro-2H-pyran-2-yl)hydroxylamine (13 mg, 0.33 mmols, 1.0 eq.) and DIPEA (0.042 mL, 0.49 mmols, 1.5 eq.) are then added and the resulting mixture left under agitation at ambient temperature for 12 hours.

At the end of the reaction (HPLC monitoring) a solution of 5% NaHCO₃ (5 ml) and AcOEt (5 ml) are added and the phases separated. The organic phase is washed with ssNaCl, dried over Na₂SO₄ and concentrated under reduced pressure. The crude product obtained is purified by flash chromatography (AcOEt) to obtain 92 mg of the desired product (70%).

¹H NMR (DMSO-d₆, 400 MHz): δ (ppm) 0.93 (2H, dq, J=3.6 and 12.0 Hz), 1.11-1.22 (2H, m), 1.28-1.44 (1H, m), 1.44-1.74 (10H, m), 1.96 (2H, t, J=7.2 Hz, CH₂CO), 2.62 (2H, m, N(CHH)₂), 3.49 (1H, m, OCHH), 3.84-4.05 (3H, m, N(CHH)₂ and OCHH), 4.21 (2H, d, J=5.6 Hz, PhCH₂), 4.79 (1H, s, OCHO), 7.00 (1H, m, NCONH), 7.16-7.33 (5H, m, ArH), 10.91 (1H, s, NHO)

MF: C₂₂H₃₃N₃O₄, MW: 403.51.

Some non-limitative examples representative of the present invention implemented in accordance with the above described synthesis schemes and variations thereof are given below.

### Ex 1 4-[1-(2-Chloro-benzenesulfonyl)-piperidin-4-yl]-N-hydroxy-butyramide

¹H NMR (DMSO-d6, 400 MHz): δ (ppm) 0.93-1.25 (4H, m), 1.25-1.41 (1H, m), 1.47 (2H, m), 1.70 (2H, d, J=12.6 Hz), 1.91 (2H, t, J=7.2 Hz, CH₂CO), 2.67 (2H, m, N(CHH)₂), 3.70 (2H, d, J=12.6 Hz, N(CHH)₂), 7.57 (1H, m, ArH), 7.65-7.75 (2H, m, ArH), 7.98 (1H, d, J=7.5 Hz, ArH), 8.65 (1H, s, OH), 10.32 (1H, s, NH).

MF: C₁₅H₂₁ClN₂O₄S, MW 360.85

### Ex 2 N-Hydroxy-4-[1-(4-methyl-naphthalene-2-sulfonyl)-piperidin-4-yl]-butyramide

¹H NMR (DMSO-d6, 300 MHz): δ (ppm) 1.17-1.23 (5H, m), 1.38-1.46 (2H, m), 1.63-1.69 (2H m), 1.87 (2H, t, J=7.3 Hz, CH₂CO), 2.43-2.48 (2H, m, (N(CHH)₂), 2.76 (3H, s), 3.70 (2H, d, J=12.0 Hz, (N(CHH)₂), 7.56 (1H, d, J=7.5 Hz, ArH), 7.71-7.75 (2H, m, ArH), 8.02 (1H, d, J=7.5 Hz, ArH), 8.18-8.21 (1H, m, ArH), 8.63 (1H, s OH), 8.68-8.71 (1H, m, ArH), 10.27 (1H, s, NH).

MF C₂₀H₂₆N₂O₄S, MW 390.49

### Ex 3 N-Hydroxy-4-[1-(naphthalene-2-carbonyl)-piperidin-4-yl]-butyramide

¹H NMR (DMSO-d6, 300 MHz): δ (ppm) 1.09-1.12 (2H, m), 1.14-1.24 (2H, m), 1.49-1.56 (3H m), 1.60-1.76 (2H, m), 1.94 (2H, t, J=7.3 Hz, CH₂CO), 2.88-3.14 (2H, m, (N(CHH)₂), 3.61-3.64 (1H, m, (N(CHH)₂), 4.41-4.60 (1H, m, (N(CHH)₂), 7.47-7.50 (1H, d, J=8.3 Hz, ArH), 7.56-7.61 (2H, m, ArH), 7.95-8.02 (4H, m, ArH), 8.66 (1H, s, OH), 10.34 (1H, s).

MF C₂₀H₂₄N₂O₃, MW 340.41

### Ex 4 N-Hydroxy-4-[1-(naphthalene-1-carbonyl)-piperidin-4-yl]-butyramide

¹H NMR (DMSO-d6, 300 MHz): δ (ppm) 0.75-1.24 (3H, m), 1.39-1.61 (4H, m), 1.76-1.87 (1H, m), 1.94 (2H, t, J=7.3 Hz, CH₂CO), 2.80-3.00 (2H, m), 3.18-3.21 (1H, m, (N(CHH)₂), 3.61-3.64 (1H, m, (N(CHH)₂), 4.63-4.70 (1H, m, (N(CHH)₂), 7.37-8.02 (7H, m, ArH), 8.66 (1H, s, OH), 10.34 (1H, s, NH).

MF C₂₀H₂₄N₂O₃, MW 340.41

### Ex 5 N-Hydroxy-4-[1-(2-naphthalen-1-yl-acetyl)-piperidin-4-yl]-butyramide

¹H NMR (DMSO-d6, 400 MHz): δ (ppm) 0.86-1.04 (2H, m), 1.11-1.20 (2H, m), 1.40-1.70 (5H, m), 1.94 (2H, t, J=7.5, Hz, CH₂CO), 2.57 (1H, t, J=11.6 Hz, NCHH), 3.04 (1H, t, J=11.6 Hz, N(CHH),), 4.00 (1H, d, J=12.9 Hz, NCHH), 4.15 (2H, s, CH₂Ar), 4.40 (1H, d, J=12.9 Hz, NCHH), 7.32-7.99 (7H, m, ArH), 8.66 (1H, s, OH), 10.33 (1H, s, NH).

MF: C₂₁H₂₆N₂O₃, MW: 354.44

### Ex 6 N-Hydroxy-4-[1-(1-naphthalen-1-yl-acetyl)-piperidin-4-yl]-butyramide

¹H NMR (DMSO-d₆, 400 MHz): δ (ppm) 0.80-1.00 (2H, m), 1.11-1.20 (2H, m), 1.40-1.70 (5H, m), 1.91 (2H, t, J=7.1 Hz, CH₂CO), 2.53 (1H, t, J=11.6 Hz, NCHH), 2.97 (1H, t, J=11.6 Hz, NCHH), 3.88 (2H, s, CH₂Ar), 4.00 (1H, d, J=12.9 Hz, NCHH), 4.40 (1H, d, J=12.9 Hz, NCHH), 7.39 (1H, d, J=8.5 Hz, ArH), 7.44-7.55 (2H, m, ArH), 7.74 (1H, s), 7.83-7.93 (3H, m), 8.64 (1H, s, OH), 10.31 (1H, s, NH).

MF C₂₁H₂₆N₂O₃, MW 354.44

### Ex 7 4-(3-Hydroxycarbamoyl-propyl)-piperidine-1-carboxylic acid benzylamide

¹H NMR (DMSO-d₆, 400 MHz): δ (ppm) 0.95 (2H, dq, J=3.4 and 12.0 Hz), 1.12-1.23 (2H, m), 1.30-1.45 (1H, m), 1.51 (2H, m), 1.56-1.66 (2H, m), 1.94 (2H, t, J=7.0 Hz, CH₂CO), 2.64 (2H, m, N(CHH)₂), 3.93-4.03 (2H, m, N(CHH)₂), 4.23 (2H, d, J=5.8 Hz, PhCH₂), 6.99 (1H, t, J=7.0 Hz, NCONH), 7.17-7.34 (5H, m, ArH), 8.65 (1H, s, OH), 10.34 (1H, s, NH).

MF C₁₇H₂₅N₃O₃, MW 319.39

### Ex 8 4-(3-Hydroxycarbamoyl-propyl)-piperidine-1-carboxylic acid phenylamide

¹H NMR (DMSO-d₆, 300 MHz): δ (ppm) 0.86-0.93 (2H, m), 1.13-1.17 (3H, m), 1.47-1.49 (2H, m), 1.61-1.65 (2H, m), 1.92 (2H, t, J=6.9 Hz, CH₂CO), 2.96 (2H, m, N(CHH)₂), 3.93 (1H, d, J=12.9 Hz, NCHH), 4.36 (1H, d, J=12.9 Hz, NCHH), 7.19 (1H, d, J=6.9 Hz, NCONH), 7.30-7.40 (5H, m, ArH), 8.66 (1H, s, OH), 10.34 (1H, s, NH).

MF C₁₆H₂₃NO₃, MW 305.37

### Ex 9 N-Hydroxy-4-[1-(naphthalene-2-sulfonyl)-piperidin-4-yl]-butyramide

¹H NMR (DMSO-d₆, 300 MHz): 6 ?(ppm) 1.04-1.28 (5H, m), 1.42 (2H, m), 1.66 (2H, d, J=12.3 Hz), 1.86 (2H, t, J=6.9 Hz, CH₂CO), 2.46 (2H, m, N(CHH)₂), 3.73 (2H, d, J=12.3 Hz, N(CHH)₂), 7.68-7.72 (3H, m, ArH), 8.12 (2H, t, J=6.5 Hz, ArH), 8.29 (1H, d, J=8.1 Hz, ArH), 8.64 (1H, d, J=6.5 Hz, ArH), 8.68 (1H, s, OH), 10.28 (1H, s, NH).

MF, C₁₉H₂₄N₂O₄S, MW 376.47

### Ex 10 N-Hydroxy-4-[1-(naphthalene-1-sulfonyl)-piperidin-4-yl]-butyramide

¹H NMR (DMSO-d₆, 300 MHz): δ (ppm) 1.12 (5H, m), 1.42 (2H, m), 1.67-1.70 (2H, m), 1.86 (2H, t, J=6.9 Hz, CH₂CO), 2.22 (2H, m, N(CHH)₂), 3.70 (2H, d, J=10.8 Hz, N(CHH)₂), 7.70-7.77 (3H, m, ArH), 8.09 (1H, d, J=8.1 Hz, ArH), 8.16-8.23 (2H, m, ArH), 8.64 (1H, s, ArH), 8.63 (1H, s, OH), 10.27 (1H, s, NH).

MF C₁₉H₂₄N₂O₄S, MW 376.47

### Ex 11 N-{3-[4-(3-Hydroxycarbamoyl-propyl)-piperidine-1-carbonyl]-phenyl}-benzamide

¹H NMR (DMSO-d₆, 400 MHz): δ (ppm) 0.96-1.16 (2H, m), 1.16-1.32 (2H, m), 1.41-1.58 (3H, m), 1.58-1.83 (2H, m), 1.94 (2H, t, J=7.2 Hz, CH₂CO), 2.62-2.83 (1H, m, NCHH), 2.92-3.12 (1H, m, NCHH), 3.57-3.70 (1H, m, NCHH), 4.40-4.54 (1H, m, NCHH), 7.09 (1H, d, J=7.6 Hz, ArH), 7.42 (1H, d, J=7.6 Hz, ArH), 7.51-7.59 (2H, m, ArH), 7.59-7.66 (1H, m, ArH), 7.81-7.88 (2H, m, ArH), 7.97 (2H, d, J=6.8 Hz, ArH), 8.69 (1H, s, OH), 10.39 (1H, s, NH).

MF C₂₃H₂₇N₃O₄, MW 409.48

### Ex 12 4-[1-(Benzofuran-2-carbonyl)-piperidin-4-yl]-N-hydroxy-butyramide

¹H NMR (DMSO-d₆, 300 MHz): δ (ppm) 1.03-1.34 (4H, m), 1.50-1.59 (3H m), 1.76 (2H, d, J=12.6 Hz), 1.94 (2H, t, J=7.3 Hz), 2.83 (1H, brs), 4.17-4.29 (1H, brs), 4.29-4.50 (1H, brs), 7.32-7.36 (2H, m, ArH), 7.44 (1H, m, ArH), 7.67 (1H, d, ArH, J=8.4 Hz), 8.66 (1H, s, OH), 10.34 (1H, s, NH).

MF C₁₈H₂₂N₂O₄, MW 330.39

### Ex 13 N-Hydroxy-4-[1-(6-methoxy-naphthalene-2-carbonyl)-piperidin-4-yl]-butyramide

¹H NMR (DMSO-d₆, 400 MHz): δ (ppm) 1.18-1.26 (4H, m), 1.48-1.82 (5H, m), 1.94 (2H, t, J=7.2 Hz, CH₂CO), 2.70-3.20 (2H, m), 3.60-3.75 (1H, m, N(CHH)₂), 3.90 (3H, s, OCH₃), 4.40-4.60 (1H, m, N(CHH)₂), 7.24 (1H, d, J=7.2 Hz, ArH), 7.38 (1H, s, ArH), 7.45 (1H, d, J=7.2 Hz, ArH), 7.85-7.95 (3H, m, ArH), 8.65 (1H, s, OH), 10.32 (1H, s, NH).

MF C₂₁H₂₆N₂O₄, MW: 370.44

### Ex 14 4-(3-Hydroxycarbamoyl-propyl)-piperidine-1-carboxylic acid (3-fluoro-phenyl)-amide

¹H NMR (DMSO-d₆, 400 MHz): δ (ppm) 0.95-1.25 (4H, m), 1.40-1.60 (4H, m), 1.68 (1H, d, J=12.3 Hz), 1.95 (2H, t, J=7.2 Hz, CH₂CO), 2.75 (2H, t, J=12.3 Hz, N(CHH)₂), 4.10 (2H, d, J=13.1 Hz, N(CHH)₂), 6.72 (1H, m, ArH), 7.25 (2H, m, ArH), 7.45 (1H, d, J=12.2 Hz, ArH), 8.61 (2H, s, OH, NHAr), 10.31 (1H, s, NH).

MF C₁₆H₂₂FN₃O₃, MW: 322.36

### Ex 15 N-Hydroxy-4-[1-(2-methyl-naphthalene-1-carbonyl)-piperidin-4-yl]-butyramide

¹H NMR (DMSO-d₆, 400 MHz): δ (ppm) 0.40-1.30 (4H, m), 1.22-1.60 (4H, m), 1.85 (1H, d, J=13.4 Hz), 1.92 (2H, t, J=7.2 Hz, CH₂CO), 2.30 (3H, s, CH₃), 2.70-3.40 (3H, m, N(CHH)₂), 4.70 (1H, t, J=13.4 Hz, N(CHH)₂), 7.40-7.58 (3H, m, ArH), 7.65 (1H, d, J=8.4 Hz, ArH), 7.68 (1H, d, J=8.4 Hz, ArH), 7.84 (1H, d, J=8.4 Hz, ArH), 8.61 (1H, s, OH), 10.29 (1H, s, NH).

MF: C₂₁H₂₆N₂O₃, MW: 354.44

### Ex 16 4-(3-Hydroxycarbamoyl-propyl)-piperidine-1-carboxylic acid (4-bromo-phenyl)-amide

¹H NMR (DMSO-d₆, 400 MHz): δ (ppm) 0.95-1.22 (4H, m), 1.40-1.60 (4H, m), 1.68 (1H, m), 1.95 (2H, t, J=7.2 Hz, CH₂CO), 2.75 (2H, t, J=12.1 Hz, N(CHH)₂), 4.10 (2H, d, J=13.2 Hz, N(CHH)₂), 7.35 (2H, d, J=8.9 Hz, ArH), 7.42 (2H, d, J=8.9 Hz, ArH), 8.60 (1H, m, NHAr), 8.54 (1H, s, OH), 10.31 (1H, s, NH).

MF: C₁₆H₂₂BrN₃O₃, MW: 384.27

### Ex 17 4-(3-Hydroxycarbamoyl-propyl)-piperidine-1-carboxylic acid (4-chloro-phenyl)-amide

¹H NMR (DMSO-d₆, 400 MHz): δ (ppm) 1.02 (2H, dq, J=3.2 and 12.4 Hz), 1.14-1.29 (2H, m), 1.37-1.48 (1H, m) 1.53 (2H, quin, J=7.4 Hz), 1.62-1.72 (2H, m), 1.95 (2H, t, J=7.2 Hz, CH₂CO), 2.75 (2H, m, N(CHH)₂), 4.10 (2H, m, N(CHH)₂), 7.26 (2H, d, J=8.8 Hz, ArH), 7.50 (2H, d, J=8.8 Hz, ArH), 8.54 (1H, m, NCONH), 8.63 (1H, bs, OH), 10.31 (1H, bs, NHOH).

MF C₁₆H₂₂ClN₃O₃, MW 339.82

### Ex 18 4-(3-Hydroxycarbamoyl-propyl)-piperidine-1-carboxylic acid (3-chloro-phenyl)-amide

MF C₁₆H₂₂ClN₃O₃, MW 339.82

### Ex 19 4-(3-Hydroxycarbamoyl-propyl)-piperidine-1-carboxylic acid (3-chloro-phenyl)-amide

MF C₁₆H₂₂FN₃O₃, MW 323.36

### Ex 20 4-(3-Hydroxycarbamoyl-propyl)-piperidine-1-carboxylic acid (4-methoxy-phenyl)-amide

¹H NMR (DMSO-d₆, 400 MHz): δ (ppm) 1.02 (2H, dq, J=3.6 and 12.0 Hz), 1.12-1.28 (2H, m), 1.34-1.47 (1H, m) 1.53 (2H, quin, J=7.4 Hz), 1.60-1.71 (2H, m), 1.95 (2H, t, J=7.2 Hz, CH₂CO), 2.72 (2H, m, N(CHH)₂), 3.71 (3H, s, OCH₃), 4.08 (2H, m, N(CHH)₂), 6.81 (2H, d, J=9.2 Hz, ArH), 7.33 (2H, d, J=9.2 Hz, ArH), 8.24 (1H, m, NCONH), 8.63 (1H, bs, OH), 10.31 (1H, bs, NHOH).

MF C₁₇H₂₅N₃O₄, MW 335.40

### Ex 21 4-(3-Hydroxycarbamoyl-propyl)-piperidine-1-carboxylic acid (3-methoxy-phenyl)-amide

MF C₁₇H₂₅N₃O₄, MW 335.40

### Ex 22 4-(3-Hydroxycarbamoyl-propyl)-piperidine-1-carboxylic acid p tolylamide

¹H NMR (DMSO-d₆, 400 MHz): δ (ppm) 1.02 (2H, dq, J=3.2 and 12.0 Hz), 1.12-1.28 (2H, m), 1.33-1.47 (1H, m) 1.53 (2H, quin, J=7.6 Hz), 1.61-1.71 (2H, m), 1.95 (2H, t, J=7.2 Hz, CH₂CO), 2.23 (3H, s, CH₃), 2.73 (2H, m, N(CHH)₂), 4.09 (2H, m, N(CHH)₂), 7.02 (2H, d, J=8.0 Hz, ArH), 7.33 (2H, d, J=8.0 Hz, ArH), 8.30 (1H, m, NCONH), 8.63 (1H, bs, OH), 10.31 (1H, bs, NHOH).

MF C₁₇H₂₅N₃O₃, MW 319.40

### Ex 23 4-(3-Hydroxycarbamoyl-propyl)-piperidine-1-carboxylic acid o-tolylamide

MF C₁₇H₂₅N₃O₃, MW 319.40

### Ex 24 4-[1-(Benzo[b]thiophene-2-carbonyl)-piperidin-4-yl]-N-hydroxy-butyramide

¹H NMR (DMSO-d₆, 300 MHz): δ (ppm) 0.86-0.91 (2H, m), 1.08-1.40 (3H m), 1.50-1.59 (2H, m), 1.75 (1H, d, J=12.1 Hz), 1.96 (2H, t, J=7.3 Hz), 2.91 (3H, brs, N(CHH)₂), 4.27 (2H, brs), 7.41-7.46 (1H, m, ArH), 7.68 (1H, s, ArH), 7.91-8.04 (2H, m, ArH), 8.63 (1H, s, OH), 10.34 (1H, s, NH).

MF C₁₈H₂₂N₂O₃S, MW 346.44

### Ex 25 4-(3-Hydroxycarbamoyl-propyl)-piperidine-1-carboxylic acid (3-fluoro-phenyl)-amide

¹H NMR (DMSO-d₆, 400 MHz): δ (ppm) 0.95-1.25 (4H, m), 1.40-1.60 (4H, m), 1.68 (1H, d, J=12.3 Hz), 1.95 (2H, t, J=7.2 Hz, CH₂CO), 2.75 (2H, t, J=12.3 Hz, N(CHH)₂), 4.10 (2H, d, J=13.1 Hz, N(CHH)₂), 6.72 (1H, m, ArH), 7.25 (2H, m, ArH), 7.45 (1H, d, J=12.2 Hz, ArH), 8.61 (2H, s, OH, NHAr), 10.31 (1H, s, NH).

MF: C₁₆H₂₂FN₃O₃, MW: 322.363

### Ex 26 4-[1-(Benzyloxycarbonyl)-piperidin-4-yl]-N-hydroxy-butyramide

¹H NMR (DMSO-d₆, 400 MHz): δ (ppm) 0.93-1.03 (2H, m), 1.15-1.51 (5H, m), 1.66 (2H, d, J=13.2 Hz), 1.93 (2H, t, J=7.2 Hz), 2.78 (2H, brs), 3.99 (2H, d, J=13.1 Hz), 5.07 (2H, s, CH₂Ph), 7.36 (5H, s, ArH), 8.62 (1H, s, OH), 10.31 (1H, s, NH).

MF: C₁₇H₂₄N₂O₄, MW: 320.38

### Ex 27 N-Hydroxy-4-[1-(3-phenyl-acryloyl)-piperidin-4-yl]-butyramide

¹H NMR (DMSO-d₆, 400 MHz): δ (ppm) 0.95-1.25 (4H, m), 1.49-1.57 (3H, m), 1.66-1.75 (2H, m), 1.95 (2H, t, J=7.2 Hz, CH₂CO), 2.62 (1H, t, J=13.3 Hz, N(CHH)₂), 3.06 (1H, t, J=12.8 Hz, N(CHH)₂), 4.47 (1H, d, J=12.7 Hz, N(CHH)₂), 4.26 (1H, d, J=11.7 Hz, N(CHH)₂), 7.23-7.27 (2H, m, ArH), 7.37-7.48 (4H, m, ArH), 7.70-7.72 (2H, m, ArH), 8.63 (1H, s, OH), 10.31 (1H, s, NH).

MF: C₁₈H₂₄N₂O₃, MW: 316.39

### Ex 28 N-Hydroxy-4-[1-(1-methyl-1H-indole-3-carbonyl)-piperidin-4-yl]-butyramide

¹H NMR (DMSO-d₆, 400 MHz): δ (ppm) 1.10 (2H, q), 1.19-1.28 (2H, m), 1.49-1.57 (3H, m), 1.70 (2H, d, J=12.5 Hz), 1.95 (2H, t, J=7.2 Hz), 2.62 (2H, t, J=13.3 Hz, N(CH₂)₂), 4.26 (2H, d, J=11.7 Hz, N(CH₂)₂), 7.14 (1H, t, ArH, J=7.4 Hz), 7.22 (1H, t, ArH, J=7.22 Hz), 7.49 (1H, d, ArH, J=8.1 Hz), 7.67 (2H, m, ArH), 8.63 (1H, s, OH), 10.31 (1H, s, NH).

MF C₁₉H₂₅N₃O₃, MW: 343.42

### Ex 29 N-Hydroxy-4-[1-(4-phenyl-butyryl)-piperidin-4-yl]-butyramide

¹H NMR (DMSO-d₆, 400 MHz): δ (ppm) 0.70-1.25 (4H, m), 1.40-1.70 (7H, m), 1.79 (2H, q, J=7.6 Hz, CH₂CH₂CH₂), 1.95 (2H, t, J=7.2 Hz, CH₂CO), 2.3 (2H, t, J=7.3 Hz, CH₂CO), 2.6 (2H, t, J=7.3 Hz, CH₂Ar), 2.91 (1H, t, J=13.0 Hz, N(CHH)₂), 3.75 (1H, d, J=13.0 Hz, N(CHH)₂), 4.40 (1H, d, J=13.0 Hz, N(CHH)₂), 7.15-7.22 (5H, m, ArH), 10.31 (1H, s, NH).

MF: C₁₉H₂₈N₂O₃, MW: 332.43

### Ex 30 N-Hydroxy-4-[1-(2-1H-indol-3-yl-acetyl)-piperidin-4-yl]-butyramide

¹H NMR (DMSO-d₆, 400 MHz): δ (ppm) 0.70-1.23 (4H, m), 1.30-1.70 (6H, m), 1.95 (2H, t, J=7.2 Hz, CH₂CO), 2.90 (1H, t, J=12.4 Hz, N(CHH)₂), 3.75 (2H, s, CH₂Ind), 4.01 (1H, d, J=12.4 Hz, N(CHH)₂), 4.40 (1H, d, J=12.4 Hz, COCH₂Ind), 6.95 (1H, t, J=7.6 Hz, ArH), 7.08 (1H, t, J=7.6 Hz, ArH), 7.18 (1H, s, ArH), 7.32 (1H, d, J=7.6 Hz, ArH), 7.58 (1H, d, J=7.6 Hz, ArH), 10.31 (1H, s, NH), 10.70 (1H, s, NHInd).

MF: C₁₉H₂₅N₃O₃, MW: 332.43

### Ex 31 N-Hydroxy-4-{1-[2-(5-methyl-2-phenyl-oxazol-4-yl)-acetyl]-piperidin-4-yl}-butyramide

¹H NMR (DMSO-d6, 400 MHz): δ (ppm) 0.70-1.23 (4H, m), 1.30-1.70 (5H, m), 1.95 (2H, t, J=7.3 Hz, CH₂CO), 2.3 (3H, s, CH₃), 2.55 (1H, t, J=12.2 Hz, N(CHH)₂), 3.05 (1H, t, J=12.2 Hz, N(CHH)₂), 3.6 (2H, s, CH₂Ar), 4.00 (1H, d, J=12.2 Hz, N(CHH)₂), 4.35 (1H, d, J=12.2 Hz, N(CHH)₂), 7.40-7.55 (3H, m, ArH), 7.75-7.85 (2H, m, ArH), 10.31 (1H, s, NH).

MF: C₂₁H₂₇N₃O₄, MW: 385.45

### Ex 32 N-Hydroxy-4-{1-[2-(2-phenyl-thiazol-4-yl)-acetyl]-piperidin-4-yl}-butyramide

¹H NMR (DMSO-d6, 400 MHz): δ (ppm) 0.70-1.23 (4H, m), 1.30-1.70 (5H, m), 1.95 (2H, t, J=7.3 Hz, CH₂CO), 2.55 (1H, t, J=12.2 Hz, N(CHH)₂), 3.05 (1H, t, J=12.2 Hz, N(CHH)₂), 3.9 (2H, s, CH₂Ar), 4.05 (1H, d, J=12.2 Hz, N(CHH)₂), 4.35 (1H, d, J=12.2 Hz, N(CHH)₂), 7.40-7.55 (4H, m, ArH), 7.75-7.85 (2H, m, ArH), 8.4 (1H, s, OH), 10.31 (1H, s, NH).

MF: C₂₀H₂₅N₃O₃S, MW: 387.50

### Ex 33 4-[1-(2-Benzo[d]isoxazol-3-yl-acetyl)-piperidin-4-yl]-N-hydroxy-butyramide

¹H NMR (DMSO-d6, 400 MHz): δ (ppm) 0.70-1.23 (4H, m), 1.30-1.70 (5H, m), 1.95 (2H, t, J=7.3 Hz, CH₂CO), 2.55 (1H, t, J=12.2 Hz, N(CHH)₂), 3.05 (1H, t, J=12.2 Hz, N(CHH)₂), 4.05 (1H, d, J=12.2 Hz, N(CHH)₂), 4.15 (2H, s, CH₂Ar), 4.35 (1H, d, J=12.2 Hz, N(CHH)₂), 7.38 (1H, t, J=7.32 Hz, ArH), 7.65 (1H, t, J=7.32 Hz, ArH), 7.42 (1H, d, J=7.32 Hz, ArH), 7.58 (1H, d, J=7.32 Hz, ArH), 8.45 (1H, bs, OH), 10.31 (1H, s, NH).

MF: C₁₈H₂₃N₃O₄, MW: 387.50

### Ex 34 4-(3-Hydroxycarbamoyl-propyl)-piperidine-1-carboxylic acid 4-chloro-benzylamide

¹H NMR (DMSO-d₆, 400 MHz): δ (ppm) 0.82-1.00 (2H, m), 1.09-1.21 (2H, m), 1.28-1.43 (1H, m), 1.49 (2H, quin, J=7.6 Hz), 1.54-1.64 (2H, m), 1.91 (2H, t, J=7.2 Hz, CH₂CO), 2.62 (2H, m, N(CHH)₂), 3.94 (2H, m, N(CHH)₂), 4.18 (2H, s, PhCH₂), 7.05 (1H, m, NCONH), 7.24 (2H, d, J=8.0 Hz, ArH), 7.35 (2H, d, J=8.0 Hz, ArH), 8.62 (1H, bs, OH), 10.32 (1H, s, NHOH).

MF C₁₇H₂₄ClN₃O₃, MW 353.84

### Ex 35 4-(3-Hydroxycarbamoyl-propyl)-piperidine-1-carboxylic acid 1-naphthylamide

¹H NMR (DMSO-d₆, 400 MHz): δ (ppm) 1.02-1.18 (2H, m), 1.18-1.33 (2H, m), 1.40-1.52 (1H, m) 1.57 (2H, m), 1.66-1.77 (2H, m), 1.97 (2H, t, J=7.2 Hz, CH₂CO), 2.84 (2H, m, N(CHH)₂), 4.18 (2H, m, N(CHH)₂), 7.38-7.56 (4H, m), 7.72 (1H, t, J=8.0 Hz, ArH), 7.88-7.96 (2H, m, ArH), 8.51 (1H, s, NCONH), 8.64 (1H, bs, OH), 10.33 (1H, s, NHOH).

MF C₂₀H₂₅N₃O₃, MW 355.43

### Ex 36 4-(3-Hydroxycarbamoyl-propyl)-piperidine-1-carboxylic acid 2-naphthylamide

¹H NMR (DMSO-d₆, 400 MHz): δ (ppm) 0.97-1.12 (2H, m), 1.16-1.30 (2H, m), 1.38-1.50 (1H, m) 1.55 (2H, m), 1.63-1.75 (2H, m), 1.96 (2H, t, J=7.2 Hz, CH₂CO), 2.80 (2H, m, N(CHH)₂), 4.17 (2H, m, N(CHH)₂), 7.34 (1H, t, J=7.4 Hz, ArH), 7.42 (1H, t, J=7.4 Hz, ArH), 7.62 (1H, d, J=8.8 Hz, ArH), 7.70-7.83 (3H, m, ArH), 8.02 (1H, s, ArH), 8.64 (2H, s, NCONH and OH), 10.32 (1H, s, NHOH).

MF C₂₀H₂₅N₃O₃, MW 355.43

### Ex 37 4-(3-Hydroxycarbamoyl-propyl)-piperidine-1-carboxylic acid (2-methoxy-phenyl)-amide

MF C₁₇H₂₅N₃O₄, MW 335.39

### Ex 38 4-(3-Hydroxycarbamoyl-propyl)-piperidine-1-carboxylic acid (2-chloro-phenyl)-amide

MF C₁₆H₂₂ClN₃O₃, MW 339.82

### Ex 39 4-(3-Hydroxycarbamoyl-propyl)-piperidine-1-carboxylic acid 3-chloro-benzylamide

¹H NMR (DMSO-d6, 400 MHz): δ (ppm) 0.70-1.23 (4H, m), 1.30-1.70 (5H, m), 1.95 (2H, t, J=7.3 Hz, CH₂CO), 2.75 (2H, t, J=12.2 Hz, N(CHH)₂), 3.95 (2H, d, J=12.2 Hz, N(CHH)₂), 4.15 (2H, s, CH₂Ar), 7.00-7.38 (5H, m, ArH⁺NH amide), 8.35 (1H, bs, OH), 10.31 (1H, s, NH).

MF: C₁₇H₂₄ClN₃O₃, MW: 387.49

### Ex 40 4-[1-(2-(S)-Amino-3-phenyl-propionyl)-piperidin-4-yl]-N-hydroxy-butyramide

¹H NMR (DMSO-d6, 400 MHz): δ (ppm) −0.23-0.11, 0.61-0.76 (1H, q, J=11.7 Hz), 0.89-1.67 (8H, m), 1.95 (2H, dd J=7.3 and 15.7 Hz), 2.82-3.08 (2H, m, CH₂Ph), 3.64 (1H, d, J=13.4 Hz, N(CHH)₂), 4.35 (1H, d, J=13.1 Hz, N(CHH)₂), 4.65 (1H, dd, J=7.0 and 14.7 Hz), 7.20-7.37 (5H, m, ArH), 8.15 (3H, brs, NH₃⁺), 8.63 (1H, bs, OH), 10.31 (1H, s, NH).

MF C₁₈H₂₇N₃O₃, MW: 333.42

### Ex 41 4-[1-(2-(R)-Amino-3-phenyl-propionyl)-piperidin-4-yl]-N-hydroxy-butyramide

MF: C₁₈H₂₇N₃O₃, MW: 333.42

### Ex 42 4-(3-Hydroxycarbamoyl-propyl)-piperidine-1-carboxylic acid 2-methoxy-benzylamide

MF: C₁₈H₂₇N₃O₄, MW: 349.42

### Ex 43 4-(3-Hydroxycarbamoyl-propyl)-piperidine-1-carboxylic acid 3-methoxy-benzylamide

MF: C₁₈H₂₇N₃O₄, MW: 349.42

### Ex 44 4-(3-Hydroxycarbamoyl-propyl)-piperidine-1-carboxylic acid 4-methoxy-benzylamide

¹H NMR (DMSO-d₆, 400 MHz): δ (ppm) 0.87-1.02 (2H, m), 1.12-1.25 (2H, m), 1.30-1.45 (1H, m), 1.51 (2H, m), 1.56-1.65 (2H, m), 1.94 (2H, t, J=7.0 Hz, CH₂CO), 2.63 (2H, m, N(CHH)₂), 3.73 (3H, s, OCH₃), 3.96 (2H, m, N(CHH)₂), 4.15 (2H, d, J=5.2 Hz, PhCH₂), 6.86 (2H, d, J=8.0 Hz, ArH), 6.87 (1H, m, NCONH), 7.17 (2H, d, J=8.0 Hz, ArH), 8.62 (1H, s, OH), 10.30 (1H, s, NH).

MF C₁₋₈H₂₇N₃O₄, MW 349.42

### Ex 45 4-(3-Hydroxycarbamoyl-propyl)-piperidine-1-carboxylic acid 2-fluoro-benzylamide

¹H NMR (DMSO-d₆, 400 MHz): δ (ppm) 0.89-1.05 (2H, m), 1.13-1.26 (2H, m), 1.32-1.46 (1H, m), 1.52 (2H, quin, J=7.4 Hz), 1.56-1.70 (2H, m), 1.94 (2H, t, J=7.0 Hz, CH₂CO), 2.66 (2H, m, N(CHH)₂), 3.97 (2H, m, N(CHH)₂), 4.28 (2H, d, J=5.2 Hz, PhCH₂), 6.95 (1H, m, NCONH), 7.09-7.20 (2H, m, ArH), 7.23-7.34 (2H, m, ArH), 8.65 (1H, s, OH), 10.31 (1H, s, NH).

MF C₁₇H₂₄FN₃O₃, MW 337.39

### Ex 46 4-(3-Hydroxycarbamoyl-propyl)-piperidine-1-carboxylic acid 3-fluoro-benzylamide

MF C₁₇H₂₄FN₃O₃, MW 337.39

### Ex 47 4-(3-Hydroxycarbamoyl-propyl)-piperidine-1-carboxylic acid 2-chloro-benzylamide

MF C₁₇H₂₄ClN₃O₃, MW 353.84

### Ex 48 4-(3-Hydroxycarbamoyl-propyl)-piperidine-1-carboxylic acid (4-dimethylamino-phenyl)-amide

¹H NMR (DMSO-d₆, 400 MHz): δ (ppm) 0.93-1.08 (2H, m), 1.13-1.34 (2H, m), 1.34-1.47 (1H, m), 1.53 (2H, m), 1.60-1.71 (2H, m), 1.95 (2H, t, J=7.0 Hz, CH₂CO), 2.71 (2H, m, N(CHH)₂), 2.84 (6H, s, 2×CH₃), 4.08 (2H, m, N(CHH)₂), 6.50-6.87 (2H, m, ArH), 7.18-7.42 (2H, m, ArH), 8.15 (1H, m, NCONH), 8.63 (1H, bs, OH), 10.35 (1H, s, NH). MF, C₁₈H₂₈N₄O₃ MW 348.44

### Ex 49 4-(3-Hydroxycarbamoyl-propyl)-piperidine-1-carboxylic acid (4-amino-phenyl)-amide

MF C₁₆H₂₄N₄O₃, MW 320.38

### Ex 50 4-(3-Hydroxycarbamoyl-propyl)-piperidine-1-carboxylic acid 2-methyl-benzylamide

MF C₁₈H₂₇N₃O₃, MW 333.42

### Ex 51 4-(3-Hydroxycarbamoyl-propyl)-piperidine-1-carboxylic acid 3-methyl-benzylamide

MF C₁₈H₂₇N₃O₃, MW 333.42

### Ex 52 4-(3-Hydroxycarbamoyl-propyl)-piperidine-1-carboxylic acid 4-methyl-benzylamide

¹H NMR (DMSO-d₆, 400 MHz): δ (ppm) 0.95 (2H, dq, J=3.2 and 12.0 Hz), 1.12-1.26 (2H, m), 1.30-1.44 (1H, m), 1.51 (2H, quin, J=7.4 Hz), 1.55-1.66 (2H, m), 1.94 (2H, t, J=7.0 Hz, CH₂CO), 2.27 (3H, s, CH₃), 2.63 (2H, m, N(CHH)₂), 3.96 (2H, m, N(CHH)₂), 4.18 (2H, d, J=4.8 Hz, PhCH₂), 6.91 (1H, m, NCONH), 7.03-7.17 (4H, m, ArH), 8.60 (1H, bs, OH), 10.30 (1H, s, NH).

MF C₁₈H₂₇NO₃, MW 333.42

### Ex 53 4-(3-Hydroxycarbamoyl-propyl)-piperidine-1-carboxylic acid (pyridin-4-ylmethyl)-amide

¹H NMR (DMSO-d₆, 400 MHz): δ (ppm) 0.90-1.05 (2H, m), 1.11-1.27 (2H, m), 1.32-1.46 (1H, m), 1.53 (2H, m), 1.58-1.69 (2H, m), 1.95 (2H, t, J=7.0 Hz, CH₂CO), 2.70 (2H, m, N(CHH)₂), 3.97 (2H, m, N(CHH)₂), 4.41 (2H, d, J=5.2 Hz, PhCH₂), 7.26 (1H, t, J=7.0 Hz, NCONH), 7.72 (2H, d, J=5.4 Hz, ArH), 8.74 (2H, d, J=5.4 Hz, ArH), 10.31 (1H, s, NH).

MF C₁₆H₂₄N₄O₃, MW 320.39

### Ex 54 4-(3-Hydroxycarbamoyl-propyl)-piperidine-1-carboxylic acid (naphthalen-1-ylmethyl)-amide

¹H NMR (DMSO-d6, 400 MHz): δ (ppm) 0.95-1.23 (4H, m), 1.30-1.70 (5H, m), 1.95 (2H, t, J=7.3 Hz, CH₂CO), 2.65 (2H, t, J=12.4 Hz, N(CHH)₂), 4.0 (2H, d, J=12.4 Hz, N(CHH)₂), 4.70 (2H, s, CH₂Ar), 6.98 (1H, s, NHCH), 7.40-7.60 (4H, m, ArH), 7.80 (1H, d, J=7.88 Hz, ArH), 7.92 (1H, d, J=7.8 Hz, ArH), 8.18 (1H, d, J=7.8 Hz, ArH), 8.60 (1H, bs, OH), 10.32 (1H, s, NH).

MF C₂₁H₂₇N₃O₃, MW: 369.46

### Ex 55 4-(3-Hydroxycarbamoyl-propyl)-piperidine-1-carboxylic acid (naphthalen-2-ylmethyl)-amide

¹H NMR (DMSO-d6, 400 MHz): δ (ppm) 0.95-1.23 (4H, m), 1.30-1.70 (5H, m), 1.95 (2H, t, J=7.30 Hz, CH₂CO), 2.65 (2H, t, J=12.4 Hz, N(CHH)₂), 4.0 (2H, d, J=12.4 Hz, N(CHH)₂), 4.30 (2H, s, CH₂Ar), 7.20 (1H, s, NHCH), 7.45-7.55 (3H, m, ArH), 7.70 (1H, s, ArH) 7.88-7.85 (3H, m, ArH), 8.30 (1H, bs, OH), 10.28 (1H, s, NH).

MF C₂, H₂₇N₃O₃, MW: 369.457

### Ex 56 4-(3-Hydroxycarbamoyl-propyl)-piperidine-1-carboxylic acid (biphenyl-4-ylmethyl)-amide

¹H NMR (DMSO-d₆, 400 MHz): δ (ppm) 0.95-1.23 (4H, m), 1.30-1.65 (5H, m), 1.95 (2H, t, J=7.25 Hz, CH₂CO), 2.64 (2H, t, J=12.3 Hz, N(CHH)₂), 4.0 (2H, d, J=12.3 Hz, N(CHH)₂), 4.30 (2H, s, CH₂Ar), 7.00 (1H, s, NHCH), 7.32 (3H, t, J=8.92 Hz, ArH), 7.45 (2H, t, J=8.92 Hz, ArH), 7.60 (2H, J=7.56 Hz, ArH), 7.65 (2H, J=7.56 Hz, ArH), 8.60 (1H, bs, OH), 10.30 (1H, s, NH).

MF C₂₃H₂₉N₃O₃, MW: 395.50

### Ex 57 4-(3-Hydroxycarbamoyl-propyl)-piperidine-1-carboxylic acid 4-phenoxy-benzylamide

¹H NMR (DMSO-d₆, 400 MHz): δ (ppm) 0.95-1.23 (4H, m), 1.30-1.75 (5H, m), 1.95 (2H, t, J=7.2 Hz, CH₂CO), 2.62 (2H, t, J=12.4 Hz, N(CHH)₂), 4.0 (2H, d, J=12.4 Hz, N(CHH)₂), 4.22 (2H, S, CH₂Ar), 6.94-7.00 (5H, m, ArH), 7.12 (1H, t, J=7.5 Hz, NHCH), 7.26 (2H, m, ArH), 7.38 (2H, d, J=7.4 Hz, ArH), 8.50 (1H, bs, OH), 10.32 (1H, s, NH).

MF C₂₃H₂₉N₃O₄, MW: 411.50

### Ex 58 4-(3-Hydroxycarbamoyl-propyl)-piperidine-1-carboxylic acid phenethyl-amide

¹H NMR (DMSO-d₆, 400 MHz): δ (ppm) 0.75-1.23 (4H, m), 1.30-1.65 (5H, m), 1.95 (2H, t, J=7.30 Hz, CH₂CO), 2.60 (2H, t, J=12.5 Hz, N(CHH)₂), 2.72 (2H, t, J=7.25 Hz, CH₂Ph), 3.40 (2H, m, CH₂NH), 3.80 (2H, d, J=12.5 Hz, N(CHH)₂), 6.50 (1H, s, CH₂NH), 7.25-7.60 (5H, m, ArH), 8.50 (1H, bs, OH), 10.32 (1H, s, NH).

MF C₁₈H₂₇N₃O₃, MW, 333.42

### Ex 59 6-{[Benzyl-(2-hydroxy-ethyl)-amino]-methyl}-7-fluoro-benzo[b]thiophene-2-carboxylic acid hydroxyamide

¹H NMR (DMSO-d₆, 400 MHz): δ (ppm) 0.75-1.23 (4H, m), 1.30-1.85 (7H, m), 1.95 (2H, t, J=7.3 Hz, CH₂CO), 2.50-2.70 (4H, m, CH₂Ph and CH₂N), 3.20 (2H, m, CH₂NH), 3.95 (2H, d, J=12.6 Hz, N(CHH)₂), 6.35 (1H, s, CH₂NH), 7.25-7.60 (5H, m, ArH), 8.50 (1H, bs, OH), 10.32 (1H, s, NH).

MF C₁₉H₂₉N₃O₃, MW: 347.45

### Ex 60 4-{1-[2-Amino-3-(4-chloro-phenyl)-propionyl]-piperidin-4-yl}-N-hydroxy-butyramide

¹H NMR (DMSO-d₆, 400 MHz): δ (ppm) −0.13-0.02, 0.63-0.75 (1H, dd, 1H, J=13.0 Hz, J=24.9 Hz), 0.99-1.01 (3H, m), 1.33-1.71 (5H, m), 1.95 (2H, dd J=7.3 and 15.7 Hz), 2.55 (1H, m), 2.86-3.05 (2H, m, CH₂Ph), 3.64-3.76 (1H, m, N(CHH)₂), 4.35 (1H, d, J=13.8 Hz, N(CHH)₂), 4.61-4.68 (1H, m, N(CHH)₂), 7.23-7.27 (2H, t, ArH, J=7.7 Hz), 7.40-7.44 (2H, dd, J=6.1 Hz, J=8.2 Hz, ArH), 8.14 (3H, bs, NH₃⁺), 8.62 (1H, bs, OH), 10.31 (1H, s, NH).

MF: C₁₈H₂₆ClN₃O₃, MW: 367.87

### Ex 61 4-{1-[2-Amino-3-(3-chloro-phenyl)-propionyl]-piperidin-4-yl}-N-hydroxy-butyramide

MF C₁₈H₂₆ClN₃O₃, MW: 367.87

### Ex 62 4-{1-[2-(R)-Amino-3-(3,4-dichloro-phenyl)-propionyl]-piperidin-4-yl}-N-hydroxy-butyramide

¹H NMR (DMSO-d6, 400 MHz): δ (ppm) −0.002-0.09, 0.64-0.73 (1H, dd, 1H, J=11.3 Hz, J=24.7 Hz), 1.16-1.24 (2H, m), 1.35-1.55 (4H, m), 1.62-1.72 (2H, m), 1.95 (2H, dd, J=7.8 Hz, J=16.5 Hz), 2.55 (1H, m), 2.77-2.84 (1H, t, J=12.3 Hz), 2.90-3.04 (2H, m, CH₂Ph), 3.72-3.78, 3.82-3.89 (1H, d, J=13 Hz, N(CHH)₂), 4.35 (1H, d, J=13.3 Hz, N(CHH)₂), 4.63-4.71 (1H, m, N(CHH)₂), 7.24 (1H, dt, ArH), 7.52-7.65 (2H, m, ArH), 8.14 (3H, brs, NH₃⁺), 8.62 (1H, bs, OH), 10.31 (1H, s, NH).

MF C₁₈H₂₅Cl₂N₃O₃, MW: 402.31

### Ex 63 4-{1-[2-(R)-Amino-3-(4-methoxy-phenyl)-propionyl]-piperidin-4-yl}-N-hydroxy-butyramide

¹H NMR (DMSO-d6, 400 MHz): δ (ppm) −0.16-0.05, 0.64-0.75 (1H, dd, J=12.2 Hz, J=23.2 Hz), 0.85-1.04 (2H, m), 1.28-1.69 (6H, m), 1.88-1.94 (2H, m), 2.83-3.03 (3H, m, CH₂Ph, N(CHH)₂), 3.61 (1H, t, J=11.7 Hz, N(CHH)₂), 3.74 (3H, s, OCH₃), 4.36 (1H, d, J=12.4 Hz, N(CHH)₂), 4.58 (1H, brs, N(CHH)₂), 6.89-6.92 (2H, m, ArH), 7.11-7.17 (2H, t, ArH, J=8.9 Hz), 8.13 (3H, brs, NH₃⁺), 8.62 (1H, bs, OH), 10.31 (1H, s, NH).

MF C₁₉H₂₉N₃O₄, MW: 363.45

### Ex 64 4-[1-(2-(R)-Amino-2-methyl-3-phenyl-propionyl)-piperidin-4-yl]-N-hydroxy-butyramide

¹H NMR (DMSO-d₆, 400 MHz): δ (ppm) 1.00-1.23 (4H, m), 1.50-1.60 (2H, m, CH₂NH), 1.63 (3H, s, CH₃), 1.80 (2H, m, CH₂NH), 1.95 (2H, t, J=7.35 Hz, CH₂CO), 2.95 (2H, m, CH₂NH), 3.10 (1H, d, J=7.00 Hz, CH₂Ph), 3.30 (1H, d, J=7.00 Hz, CH₂Ph), 4.30 (2H, m, CH₂NH), 7.20-7.40 (5H, m, ArH), 8.00 (3H, s, NH₃⁺), 8.60 (1H, bs, OH), 10.32 (1H, s, NH).

MF C₁₉H₂₉N₃O₃, MW: 347.45

### Ex 65 4-{1-[2-(R)-Amino-3-(1H-indol-3-yl)-propionyl]-piperidin-4-yl}-N-hydroxy-butyramide

¹H NMR (DMSO-d₆, 400 MHz): δ (ppm) −0.50-0.30 (1H, dq, J=12.2 Hz), 0.8-1.7 (6H, m), 1.95 (2H, t, J=7.35 Hz, CH₂CO), 2.7 (1H, t, J=12.4 Hz, N(CHH)₂), 3.15 (2H, m, CH₂In), 3.50 (1H, t, J=13.2 Hz, N(CHH)₂), 3.65 (1H, t, J=13.2 Hz, N(CHH)₂), 4.30 (1H, t, J=11.9 Hz, N(CHH)₂), 4.60 (1H, m, CHNH), 7.00-7.15 (2H, dt, J=7.60 Hz, ArH), 7.25 (1H, m, ArH), 7.38 (1H, d, J=7.60 Hz, ArH) ArH), 7.45-7.55 (1H, d, J=7.60 Hz, ArH), 8.2 (3H, bs, NH₃⁺), 8.60 (1H, s, OH), 10.32 (1H, s, NH), 11.0 (1H, s, NHInd).

MF C₂₀H₂₈N₄O₃, MW: 372.46

### Ex 66 4-{1-[2-(S)-Amino-3-(1H-indol-3-yl)-propionyl]-piperidin-4-yl}-N-hydroxy-butyramide

¹H NMR (DMSO-d₆, 400 MHz): δ (ppm) −0.50-0.30 (1H, dq, J=12.25 Hz), 0.8-1.7 (6H, m), 1.95 (2H, t, J=7.35 Hz, CH₂CO), 2.7 (1H, t, J=12.4 Hz, N(CHH)₂), 3.15 (2H, m, CH₂In), 3.50 (1H, t, J=13.2 Hz, N(CHH)₂), 3.65 (1H, t, J=13.2 Hz, N(CHH)₂), 4.30 (1H, t, J=11.92 Hz, N(CHH)₂), 4.60 (1H, m, CHNH), 7.00-7.15 (2H, dt, J=7.60 Hz, ArH), 7.25 (1H, m, ArH), 7.38 (1H, d, J=7.60 Hz, ArH), 7.45-7.55 (1H, d, J=7.60 Hz, ArH), 8.2 (3H, bs, NH₃⁺), 8.60 (1H, s, OH), 10.32 (1H, s, NH), 11.0 (1H, s, NH-Ind).

MF C₂₀H₂₈N₄O₃, MW: 372.46

### Ex 67 4-[1-(2-(R)-Amino-3-benzyloxy-propionyl)-piperidin-4-yl]-N-hydroxy-butyramide

¹H NMR (DMSO-d₆, 400 MHz): δ (ppm) 0.60-1.23 (4H, m), 1.40-1.80 (5H, m), 1.95 (2H, t, J=7.27 Hz, CH₂CO), 2.65 (1H, m, CH₂NH), 2.95 (1H, m, CH₂NH), 3.60-3.70 (2H, m, OCH₂Ph), 4.50-4.70 (3H, m, CHCH₂O, CHNH₂), 3.80 (1H, m, CH₂NH), 4.35 (1H, m, CH₂NH), 7.20-7.40 (5H, m, ArH), 8.20 (3H, s, NH₃⁺), 8.60 (1H, bs, OH), 10.36 (1H, s, NH).

MF C₁₉H₂₉N₃O₄, MW: 347.45

### Ex 68 4-{1-[2-(R)-Amino-3-(4-tert-butoxy-phenyl)-propionyl]-piperidin-4-yl}-N-hydroxy-butyramide

¹H NMR (DMSO-d₆, 400 MHz): δ (ppm) 0.0-0.80 (1H, dq, J=12.23 Hz), 0.7-1.2 (2H, m), 1.30 (9H, s, O(CH₃)₃), 1.30-1.70 (5H, m), 1.95 (2H, q, J=7.80 Hz, CH₂CO), 2.2 (1H, t, J=11.0 Hz, N(CHH)₂), 2.45 (1H, t, J=11.0 Hz, N(CHH)₂), 2.80-3.00 (2H, m, CH₂Ph), 3.60 (1H, m, N(CHH)₂), 4.35 (1H, d, J=12.6 Hz, N(CHH)₂), 4.65 (1H, m, CHNH), 6.90-6.96 (2H, d, J=7.08 Hz, ArH), 7.10-7.18 (2H, d, J=7.08 Hz, ArH), 8.2 (3H, bs, NH₃⁺), 8.00 (1H, s, OH), 10.32 (1H, s, NH).

MF C₂₂H₃₅N₃O₄, MW: 372.46

### Ex 69 4-[1-(2-(R)-Amino-3-benzylsulfanyl-propionyl)-piperidin-4-yl]-N-hydroxy-butyramide

¹H NMR (DMSO-d₆, 400 MHz): δ (ppm) 0.70-1.23 (4H, m), 1.40-1.80 (5H, m), 1.95 (2H, t, J=7.27 Hz, CH₂CO), 2.65-3.10 (4H, m, CH₂NH, CH₂Ph), 3.75 (1H, t, J=12.39 Hz, CH₂NH), 3.85 (2H, d, J=8.74 Hz, SCH₂Ph), 4.35 (1H, d, J=13.36 Hz, CH₂NH), 4.55 (1H, m, CH₂NH), 7.20-7.40 (5H, m, ArH), 8.20 (3H, s, NH₃⁺), 8.60 (1H, bs, OH), 10.36 (1H, s, NH).

MF C₁₉H₂₉N₃O₃S, MW 379.517

### Ex 70 4-(3-Hydroxycarbamoyl-propyl)-piperidine-1-carboxylic acid pyridin-3-ylamide

¹H NMR (DMSO-d₆, 400 MHz): δ (ppm) 0.98-1.11 (2H, m), 1.15-1.31 (2H, m), 1.39-1.62 (3H, m), 1.62-1.76 (2H, m), 1.95 (2H, t, J=7.0 Hz, CH₂CO), 2.82 (2H, m, N(CHH)₂, 4.13 (2H, m, N(CHH)₂), 7.66 (1H, m, ArH), 8.23 (1H, d, J=8.4 Hz, ArH), 8.34 (1H, d, J=4.4 Hz, ArH), 8.91 (1H, m, ArH), 9.08 (1H, s, NCONH), 10.32 (1H, s, NH).

MF C₁₅H₂₂N₄O₃, MW 306.36

### Ex 71 4-(3-Hydroxycarbamoyl-propyl)-piperidine-1-carboxylic acid (pyridin-3-ylmethyl)-amide

¹H NMR (DMSO-d₆, 400 MHz): δ (ppm) 0.95 (2H, dq, J=3.2 and 12.0 Hz), 1.11-1.23 (2H, m), 1.32-1.46 (1H, m), 1.51 (2H, quin, J=7.5 Hz), 1.56-1.71 (2H, m), 1.94 (2H, t, J=7.0 Hz, CH₂CO), 2.67 (2H, m, N(CHH)₂, 3.95 (2H, m, N(CHH)₂), 4.34 (2H, d, J=3.9 Hz, PhCH₂), 7.18 (1H, s, NCONH), 7.73-7.84 (1H, m, ArH), 8.12-8.22 (1H, m, ArH), 8.65-8.72 (2H, m, ArH), 8.46 (1H, bs, OH), 10.32 (1H, s, NH).

MF C₁₆H₂₄N₄O₃, MW 320.39

### Ex 72 4-(3-Hydroxycarbamoyl-propyl)-piperidine-1-carboxylic acid (3-amino-phenyl)-amide

MF C₁₆H₂₄N₄O₃, MW 320.39

### Ex 73 4-(3-Hydroxycarbamoyl-propyl)-piperidine-1-carboxylic acid (2-amino-phenyl)-amide

¹H NMR (DMSO-d₆, 400 MHz): δ (ppm) 1.08 (2H, dq, J=3.2 and 12.0 Hz), 1.18-1.30 (2H, m), 1.40-1.50 (1H, m), 1.55 (2H, m), 1.64-1.74 (2H, m), 1.96 (2H, t, J=7.0 Hz, CH₂CO), 2.82 (2H, m, N(CHH)₂, 4.12 (2H, m, N(CHH)₂), 7.09-7.25 (4H, m, ArH), 8.44 (1H, s, OH), 10.31 (1H, s, NH).

MF C₁₆H₂₄N₄O₃, MW 320.39

### Ex 74 4-(3-Hydroxycarbamoyl-propyl)-piperidine-1-carboxylic acid 4-dimethylamino-benzylamide

¹H NMR (DMSO-d₆, 400 MHz): δ (ppm) 0.94 (2H, dq, J=3.2 and 12.0 Hz), 1.12-1.23 (2H, m), 1.30-1.45 (1H, m), 1.51 (2H, quin, J=7.6 Hz), 1.56-1.65 (2H, m), 1.94 (2H, t, J=7.0 Hz, CH₂CO), 2.62 (2H, m, N(CHH)₂), 2.95 (6H, s, 2×CH₃), 3.96 (2H, m, N(CHH)₂), 4.15 (2H, d, J=5.8 Hz, PhCH₂), 6.90 (1H, s, NCONH), 7.19 (4H, m, ArH), 10.31 (1H, s, NH).

MF C₁₉H₃₀N₄O₃, MW 362.47

### Ex 75 4-(3-Hydroxycarbamoyl-propyl)-piperidine-1-carboxylic acid (4-fluoro-phenyl)-amide

MF C₁₆H₂₂FN₃O, MW 323.36

### Ex 76 4-(3-Hydroxycarbamoyl-propyl)-piperidine-1-carboxylic acid m-tolylamide

MF C₁₇H₂₅N₃O₃, MW 319.40

### Ex 77 4-(3-Hydroxycarbamoyl-propyl)-piperidine-1-carboxylic acid benzyl-methyl-amide

¹H NMR (DMSO-d₆, 400 MHz): δ (ppm) 0.95 (2H, dq, J=3.2 and 12.4 Hz), 1.14-1.24 (2H, m), 1.31-1.45 (1H, m), 1.51 (2H, quin, J=7.4 Hz), 1.58-1.69 (2H, m), 1.94 (2H, t, J=7.0 Hz, CH₂CO), 2.63-2.74 (2H, m, N(CHH)₂), 2.67 (3H, s, CH₃), 3.57 (2H, m, N(CHH)₂), 4.31 (2H, s, PhCH₂), 7.22-7.30 (3H, m, ArH), 7.32-7.39 (2H, m, ArH), 8.63 (1H, s, OH), 10.31 (1H, s, NH).

MF C₁₈H₂₇NO₃, MW 333.42

### Ex 78 N-Hydroxy-4-{1-[2-(2-1H-indol-3-yl-acetylamino)-acetyl]-piperidin-4-yl}-butyramide

¹H NMR (DMSO-d6, 400 MHz): δ (ppm) 0.70-1.20 (4H, m), 1.40-1.70 (5H, m), 1.95 (2H, t, J=7.27 Hz, CH₂CO), 2.55 (1H, t, J=12.3 Hz, CH₂NH), 2.95 (1H, t, J=12.3 Hz, CH₂NH), 3.58 (2H, s, CH₂Ind), 3.70 (1H, d, J=12.3 Hz, CH₂NH), 3.80-3.95 (2H, m, CH₂NH), 4.30 (1H, d, J=12.3 Hz, CH₂NH), 6.95-7.10 (2H, dt, J=7.36 Hz, ArH), 7.25 (1H, s, ArH), 7.35 (1H, d, J=7.4 Hz, ArH), 7.55 (1H, d, J=7.4 Hz, ArH), 7.80 (1H, m, CH₂NH), 8.60 (1H, bs, OH), 10.36 (1H, s, NH), 10.85 (1H, s, NHInd).

MF: C₂₁H₂₈N₄O₄, MW: 400.47

### Ex 79 Benzo[b]thiophene-2-carboxylic acid {2-[4-(3-hydroxycarbamoyl-propyl)-piperidin-1-yl]-2-oxo-ethyl}-amide

¹H NMR (DMSO-d₆, 400 MHz): δ (ppm) 0.70-1.20 (4H, m), 1.40-1.80 (5H, m), 1.95 (2H, t, J=7.30 Hz, CH₂CO), 2.55 (1H, t, J=10.9 Hz, CH₂NH), 3.00 (1H, t, J=10.9 Hz, CH₂NH), 3.90 (1H, d, J=10.9 Hz, CH₂NH), 4.15 (2H, s, CH₂Gly), 4.40 (1H, d, J=10.9 Hz, CH₂NH), 7.40-7.50 (2H, m, ArH), 7.95-8.15 (2H, d, J=7.4 Hz, ArH), 8.65 (1H, s, OH), 8.65 (1H, s, NHCH), 10.36 (1H, s, NH).

MF: C₂₀H₂₅N₃O₄S, MW: 403.49

### Ex 80 Naphthalene-1-carboxylic acid {2-[4-(3-hydroxycarbamoyl-propyl)-piperidin-1-yl]-2-oxo-ethyl}-amide

¹H NMR (DMSO-d₆, 400 MHz): δ (ppm) 0.75-1.25 (4H, m), 1.40-1.80 (5H, m), 1.95 (2H, t, J=7.3 Hz, CH₂CO), 2.60 (1H, t, J=10.9 Hz, CH₂NH), 3.05 (1H, t, J=10.9 Hz, CH₂NH), 3.90 (1H, d, J=10.9 Hz, CH₂NH), 4.15 (2H, d, J=5.8 Hz, CH₂Gly), 4.40 (1H, d, J=10.9 Hz, CH₂NH), 7.55-7.65 (4H, m, ArH), 7.95-8.05 (2H, m, ArH), 8.50 (1H, t, J=5.6 Hz, NHCH₂), 8.65 (1H, s, OH), 10.36 (1H, s, NH).

MF: C₂₂H₂₇N₃O₄, MW: 397.47

### Ex 81 2-Methyl-naphthalene-*1-carboxylic acid {2-[4-(3-hydroxycarbamoyl-propyl)-piperidin-1-yl]-2-oxo-ethyl}-amide

¹H NMR (DMSO-d₆, 400 MHz): δ (ppm) 0.75-1.25 (4H, m), 1.40-1.80 (5H, m), 1.95 (2H, t, J=7.3 Hz, CH₂CO), 2.45 (3H, s, CH₃), 2.60 (1H, t, J=10.9 Hz, CH₂NH), 3.05 (1H, t, J=10.9 Hz, CH₂NH), 3.90 (1H, d, J=10.9 Hz, CH₂NH), 4.15 (2H, d, J=5.8 Hz, CH₂Gly), 4.40 (1H, d, J=10.90 Hz, CH₂NH), 7.55-7.65 (3H, m, ArH), 7.95-8.05 (2H, m, ArH), 8.50 (1H, s, NHCH₂), 8.65 (1H, bs, OH), 10.36 (1H, s, NH).

MF C₂₃H₂₉N₃O₄, MW: 411.49

### Ex 82 4-[1-(2-(S)-Amino-4-phenyl-butyryl)-piperidin-4-yl]-N-hydroxy-butyramide

¹H NMR (DMSO-d₆, 400 MHz): δ (ppm) 0.86-1.02 (2H, m), 1.17 (2H, m), 1.51 (3H, m), 1.69 (2H, m), 1.94 (4H, m), 2.57-2.74 (3H, m, CH₂Ph), 2.92-3.07 (1H, q, J=13.6 Hz), 3.61-3.75 (1H, m), 4.29-4.42 (2H, m), 7.21-7.32 (5H, m, ArH), 8.12 (2H, s, NH₃⁺), 8.63 (1H, s), 10.32 (1H, s, NH).

MF: C₁₇H₂₄N₂O₄, MW: 320.38

### Ex 83 4-(3-Hydroxycarbamoyl-propyl)-piperidine-1-carboxylic acid (3-chloro-2-fluoro-phenyl)-amide

¹H NMR (DMSO-d₆, 400 MHz): δ (ppm) 1.04 (2H, dq, J=3.2 and 12.0 Hz), 1.13-1.29 (2H, m), 1.37-1.48 (1H, m) 1.54 (2H, quin, J=7.4 Hz), 1.61-1.73 (2H, m), 1.95 (2H, t, J=7.2 Hz, CH₂CO), 2.78 (2H, m, N(CHH)₂), 4.06 (2H, m, N(CHH)₂), 7.12 (1H, m, ArH), 7.27 (1H, m, ArH)), 7.37 (1H, m, ArH), 8.39 (1H, s, NCONH), 8.64 (1H, bs, OH), 10.32 (1H, s, NHOH).

MF C₁₆H₂₁ClFN₃O₃, MW 357.81

### Ex 84 4-(3-Hydroxycarbamoyl-propyl)-piperidine-1-carboxylic acid (2,4-difluoro-phenyl)-amide

¹H NMR (DMSO-d₆, 400 MHz): δ (ppm) 1.03 (2H, dq, J=3.2 and 12.0 Hz), 1.14-1.29 (2H, m), 1.35-1.48 (1H, m) 1.53 (2H, m), 1.60-1.72 (2H, m), 1.95 (2H, t, J=7.2 Hz, CH₂CO), 2.76 (2H, m, N(CHH)₂), 4.05 (2H, m, N(CHH)₂), 7.00 (1H, m, ArH), 7.21 (1H, m, ArH), 7.38 (1H, m, ArH), 8.18 (1H, s, NCONH), 8.61 (1H, bs, OH), 10.32 (1H, s, NHOH).

MF C₁₆H₂₁F₂N₃O₃, MW 341.35

### Ex 85 4-(3-Hydroxycarbamoyl-propyl)-piperidine-1-carboxylic acid (3-chloro-4-methyl-phenyl)-amide

¹H NMR (DMSO-d₆, 400 MHz): δ (ppm) 1.02 (2H, dq, J=3.7 and 12.6 Hz), 1.13-1.26 (2H, m), 1.35-1.47 (1H, m) 1.53 (2H, m), 1.60-1.73 (2H, m), 1.95 (2H, t, J=7.2 Hz, CH₂CO), 2.24 (3H, s, CH₃), 2.74 (2H, m, N(CHH)₂), 4.09 (2H, m, N(CHH)₂), 7.15-7.21 (1H, m, ArH), 7.31 (1H, dd, J=2.1 and 8.3 Hz, ArH), 7.61-7.66 (1H, m, ArH), 8.50 (1H, s, OH), 10.32 (1H, s, NHOH).

MF C₁₇H₂₄ClN₃O₃, MW 353.84

### Ex 86 4-(3-Hydroxycarbamoyl-propyl)-piperidine-1-carboxylic acid (3,4-difluoro-phenyl)-amide

¹H NMR (DMSO-d₆, 400 MHz): δ (ppm) 1.02 (2H, dq, J=3.7 and 12.5 Hz), 1.14-1.27 (2H, m), 1.37-1.48 (1H, m) 1.53 (2H, m), 1.61-1.74 (2H, m), 1.95 (2H, t, J=7.2 Hz, CH₂CO), 2.76 (2H, m, N(CHH)₂), 4.09 (2H, m, N(CHH)₂), 7.17-7.34 (2H, m, ArH), 7.62 (1H, ddd, J=2.3 and 7.5 and 13.7 Hz), 8.61 (2H, bs, NCONH and OH), 10.31 (1H, s, NHOH).

MF C₁₆H₂₁F₂N₃O₃, MW 341.35

### Ex 87 4-(3-Hydroxycarbamoyl-propyl)-piperidine-1-carboxylic acid (3,4-dimethyl-phenyl)-amide

¹H NMR (DMSO-d₆, 400 MHz): δ (ppm) 1.01 (2H, dq, J=3.5 and 12.6 Hz), 1.14-1.25 (2H, m), 1.32-1.47 (1H, m) 1.53 (2H, m), 1.61-1.71 (2H, m), 1.95 (2H, t, J=7.2 Hz, CH₂CO), 2.14 (3H, s, CH₃), 2.16 (3H, s, CH₃), 2.72 (2H, m, N(CHH)₂), 4.09 (2H, m, N(CHH)₂), 6.93-6.99 (1H, m, ArH), 7.13-7.19 (1H, m, ArH), 7.21-7.25 (1H, m, ArH), 8.23 (1H, s, NCONH), 8.63 (1H, bs, OH), 10.31 (1H, s, NHOH).

MF C₁₈H₂₇N₃O₃, MW 333.42

### Ex 88 4-(3-Hydroxycarbamoyl-propyl)-piperidine-1-carboxylic acid (3-chloro-4-fluoro-phenyl)-amide

¹H NMR (DMSO-d₆, 400 MHz): δ (ppm) 1.02 (2H, dq, J=3.8 and 12.6 Hz), 1.13-1.25 (2H, m), 1.36-1.48 (1H, m) 1.53 (2H, m), 1.62-1.73 (2H, m), 1.94 (2H, t, J=7.2 Hz, CH₂CO), 2.76 (2H, m, N(CHH)₂), 4.09 (2H, m, N(CHH)₂), 7.27 (1H, d, J=9.1 Hz, ArH), 7.38-7.45 (1H, m, ArH), 7.76 (1H, dd, J=2.6 and 6.9 Hz, ArH), 8.61 (2H, bs, NCONH and OH), 10.31 (1H, s, NHOH).

MF C₁₆H₂₁ClFN₃O₃, MW 357.81

### Ex 89 4-(3-Hydroxycarbamoyl-propyl)-piperidine-1-carboxylic acid (2,5-difluoro-phenyl)-amide

¹H NMR (DMSO-d₆, 400 MHz): δ (ppm) 1.04 (2H, dq, J=3.7 and 12.6 Hz), 1.14-1.27 (2H, m), 1.35-1.48 (1H, m) 1.53 (2H, m), 1.60-1.74 (2H, m), 1.95 (2H, t, J=7.2 Hz, CH₂CO), 2.78 (2H, m, N(CHH)₂), 4.03-4.11 (2H, m, N(CHH)₂), 6.84-6.93 (1H, m, ArH), 7.17-7.26 (1H, m, ArH), 7.37-7.46 (1H, m, ArH), 8.30 (1H, s, NCONH), 8.63 (1H, s, OH), 10.33 (1H, s, NHOH).

MF C₁₆H₂₁F₂N₃O₃, MW 341.35

### Ex 90 4-(3-Hydroxycarbamoyl-propyl)-piperidine-1-carboxylic acid (4-chloro-2-fluoro-phenyl)-amide

¹H NMR (DMSO-d₆, 400 MHz): δ (ppm) 1.03 (2H, dq, J=3.8 and 12.6 Hz), 1.15-1.27 (2H, m), 1.37-1.48 (1H, m) 1.53 (2H, quin, J=7.4 Hz), 1.61-1.74 (2H, m), 1.95 (2H, t, J=7.2 Hz, CH₂CO), 2.77 (2H, m, N(CHH)₂), 4.06 (2H, m, N(CHH)₂), 7.16-7.22 (1H, m, ArH), 7.38 (1H, dd, J=2.3 and 10.5 Hz, ArH), 7.42-7.49 (1H, m, ArH), 8.27 (1H, s, NCONH), 8.62 (1H, bs, OH), 10.31 (1H, s, NHOH).

MF C₁₆H₂₁ClFN₃O₃, MW 357.81

### Ex 91 4-(3-Hydroxycarbamoyl-propyl)-piperidine-1-carboxylic acid (2,4-dimethoxy-phenyl)-amide

¹H NMR (DMSO-d₆, 400 MHz): δ (ppm) 1.03 (2H, dq, J=3.8 and 12.6 Hz), 1.14-1.28 (2H, m), 1.33-1.48 (1H, m) 1.53 (2H, m), 1.63-1.71 (2H, m), 1.95 (2H, t, J=7.2 Hz, CH₂CO), 2.74 (2H, m, N(CHH)₂), 3.74 (3H, s, OCH₃), 3.78 (3H, s, OCH₃), 4.01 (2H, m, N(CHH)₂), 6.45 (1H, dd, J=2.6 and 8.7 Hz, ArH), 6.57 (1H, d, J=2.6 Hz, ArH), 7.35-7.39 (1H, m, ArH), 7.42 (1H, s, NCONH), 8.63 (1H, bs, OH), 10.31 (1H, s, NHOH).

MF C₁₈H₂₇N₃O₅, MW 365.42

### Ex 92 N-Hydroxy-4-[1-(2-(R)-methylamino-3-phenyl-propionyl)-piperidin-4-yl]-butyramide

¹H NMR (DMSO-d₆, 400 MHz): δ (ppm) −0.5-0.60 (1H, dq, J=12.20 Hz), 0.8-1.7 (8H, m), 1.95 (2H, q, J=7.80 Hz, CH₂CO), 2.05-2.45 (1H, d7, J=13.4 Hz, N(CHH)₂), 2.5 (3H, m, CH₃), 2.70-2.85 (2H, m, CH₂Ph), 3.10 (1H, m, N(CHH)₂), 3.25 (1H, m, N(CHH)₂), 4.35 (1H, m, N(CHH)₂), 4.45 (1H, m, CHNHCH₃), 7.10-7.40 (5H, m, ArH), 8.8-9.2 (3H, bs, NH₂⁺, OH), 10.32 (1H, s, NH).

MF: C₁₉H₂₉N₃O₃, MW: 347.452

### Ex 93 4-[1-(2-(R)-Amino-3-naphthalen-1-yl-propionyl)-piperidin-4-yl]-N-hydroxy-butyramide

¹H NMR (DMSO-d₆, 400 MHz): δ (ppm) −0.5-0.65 (2H, dq, J=12.1 Hz), 0.7-1.7 (7H, m), 1.95 (2H, q, J=7.80 Hz, CH₂CO), 2.15-2.45 (1H, dt, J=13.3 Hz, N(CHH)₂), 2.75-2.85 (2H, m, CH₂Ph), 3.10 (1H, m, N(CHH)₂), 3.45 (1H, m, N(CHH)₂), 4.35 (1H, m, N(CHH)₂), 4.50 (1H, m, CHNH₂), 7.40-8.00 (7H, m, ArH), 8.2-8.5 (4H, bs, NH₃⁺, OH), 10.28 (1H, d, J=13.00 Hz, NH).

MF: C₂₂H₂₉N₃O₃, MW: 383.48

### Ex 94 4-[1-(2-(R)-Amino-3-benzo[b]thiophen-3-yl-propionyl)-piperidin-4-yl]-N-hydroxy-butyramide

¹H NMR (DMSO-d6, 400 MHz): δ (ppm) −0.57-0.12 (1H, dq), 0.73-1.79 (8H, m), 1.86 (2H, m), 2.15-2.28 (1H, m), 2.37 (1H, t, J=12.5 Hz), 2.73 (2H, m), 3.18-3.36 (2H, m), 4.26 (1H, t, J=13.7 Hz, N(CHH)₂), 4.73 (1H, s, CHNH₂), 7.43 (2H, m, ArH), 7.61 (1H, s, ArH), 7.75-7.81 (1H, 2d, J=7.4 Hz, ArH), 8.02 (1H, d, J=7.7 Hz, ArH), 8.2-8.5 (3H, bs, NH₃⁺), 8.69 (1H, brs, OH), 10.28 (1H, s, NH).

MF: C₂₀H₂₇N₃O₃S, MW: 389.51

### Ex 95 4-[1-(2-(R)-Amino-3-m-tolyl-propionyl)-piperidin-4-yl]-N-hydroxy-butyramide

¹H NMR (DMSO-d₆, 400 MHz): δ (ppm) −0.2-0.75 (1H, dq, J=12.10 Hz), 0.85-1.85 (8H, m), 1.95 (2H, q, J=7.9 Hz, CH₂CO), 2.3 (3H, s, CH₃), 2.25-2.45 (1H, m, N(CHH)₂), 2.70-2.75 (1H, m, N(CHH)₂), 2.75-2.85 (2H, m, CH₂Ph), 3.40-3.50 (1H, m, N(CHH)₂), 4.10-4.20 (1H, m, N(CHH)₂), 4.50 (1H, m, CHNH₂), 7.00-7.40 (4H, m, ArH), 8.1-8.4 (4H, bs, NH₃⁺, OH), 10.25 (1H, d, J=13.0 Hz, NH).

MF C₁₉H₂₉N₃O₃, MW: 347.45

### Ex 96 4-{1-[2-(S)-Amino-3-(4-benzoyl-phenyl)-propionyl]-piperidin-4-yl}-N-hydroxy-butyramide

¹H NMR (DMSO-d₆, 400 MHz): δ (ppm) −0.20-0.75 (1H, 2q, J=12.1 Hz), 0.87-1.06 (2H, m), 1.12-1.22 (1H, m), 1.28-1.72 (5H, m), 1.95 (2H, 2t, J=7.3 Hz), 2.45 (1H, m, N(CHH)₂), 2.90 (1H, t, J=13.5 Hz, N(CHH)₂), 3.00-3.20 (2H, m, CH₂Ph), 3.65-3.77 (1H, m, N(CHH)₂), 4.37 (1H, d, N(CHH)₂ J=12.8 Hz), 4.74 (1H, m, CHNH₂), 7.41-7.48 (2H, m, ArH), 7.57-7.61 (2H, m, ArH), 7.68-7.77 (5H, m, ArH), 8.20 (3H, d, NH₃⁺), 8.61 (1H, brs, OH), 10.29 (1H, d, J=9.9 Hz, NH).

MF C₂₅H₃₁N₃O₄, MW: 437.53

### Ex 97 4-{1-[2-(R)-Amino-3-(2-fluoro-phenyl)-propionyl]-piperidin-4-yl}-N-hydroxy-butyramide

¹H NMR (DMSO-d₆, 400 MHz): δ (ppm) 0.0-0.80 (1H, dq, J=12.1 Hz), 0.85-1.85 (8H, m), 1.95 (2H, q, J=7.9 Hz, CH₂CO), 2.35-2.45 (1H, m, N(CHH)₂), 2.60-2.65 (1H, m, N(CHH)₂), 2.75-2.85 (2H, m, CH₂Ph), 3.40-3.50 (1H, m, N(CHH)₂), 4.15 (1H, d, J=11.9 Hz, N(CHH)₂), 4.55 (1H, m, CHNH₂), 7.10-7.50 (4H, m, ArH), 8.10-8.35 (4H, bs, NH₃⁺, OH), 10.20 (1H, d, J=13.0 Hz, NH).

MF C₁₈H₂₆FN₃O₃, MW: 351.42

### Ex 98 4-{1-[2-(R)-Amino-3-(3-fluoro-phenyl)-propionyl]-piperidin-4-yl}-N-hydroxy-butyramide

MF C₁₈H₂₆FN₃O₃, MW: 351.42

### Ex 99 4-{1-[2-(R)-Amino-3-(4-fluoro-phenyl)-propionyl]-piperidin-4-yl}-N-hydroxy-butyramide

MF C₁₈H₂₆FN₃O₃, MW: 351.42

### Ex 100 N-Hydroxy-4-[1-(2-(R)-hydroxy-3-phenyl-propionyl)-piperidin-4-yl]-butyramide

¹H NMR (DMSO-d₆, 400 MHz): δ (ppm) 0.75-1.20 (4H, m), 1.40-1.80 (5H, m), 1.95 (2H, t, J=7.30 Hz, CH₂CO), 2.50 (1H, m, CH₂NH), 2.70 (1H, m, CH₂NH), 2.85 (2H, m, CH₂Ph), 3.90 (1H, t, J=12.3 Hz, CH₂NH), 4.35 (1H, m, CH₂NH), 4.40 (1H, d, J=10.9 Hz, CH₂NH), 4.50 (1H, m, CHOH), 7.15-7.30 (5H, m, ArH), 8.65 (1H, bs, OH), 10.35 (1H, s, NH).

MF C₁₈H₂₆N₂O₄, MW: 334.41

### Ex 101 Benzo[b]thiophene-2-carboxylic acid {2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.70-1.20 (4H, m), 1.40-1.80 (5H, m), 1.95 (2H, m, CH₂CO), 2.55 (1H, t, J=12.5 Hz, CH₂NH), 3.05 (1H, t, J=12.5 Hz, CH₂NH), 3.60 (1H, m, CH₂OH), 3.75 (1H, m, CH₂OH), 4.05 (1H, d, J=12.3 Hz, CH₂NH), 4.40 (1H, d, J=12.3 Hz, CH₂NH), 4.90 (1H, m, CH₂OH), 5.00 (1H, m, CHCH₂OH), 7.40-7.50 (1H, m, ArH), 7.95 (1H, d, J=7.2 Hz, ArH), 8.05 (1H, d, J=7.2 Hz, ArH), 8.20-8.25 (1H, m, ArH), 8.65 (1H, bs, OH), 8.80 (1H, m, NHCH), 10.35 (1H, s, NH).

MF C₂₁H₂₇N₃O₅S, MW: 433.52

### Ex 102 Benzo[b]thiophene-2-carboxylic acid {2-[4-(3-hydroxycarbamoyl-propyl)-piperidin-1-yl]-1-(R)-hydroxymethyl-2-oxo-ethyl}-amide

MF C₂₁H₂₇N₃O₅S, MW: 433.52

### Ex 103 4-[1-(2-(S)-Dimethylamino-4-phenyl-butyryl)-piperidin-4-yl]-N-hydroxy-butyramide

¹H NMR (DMSO-d₆, 400 MHz): δ (ppm) 0.70-1.20 (4H, m), 1.40-1.80 (7H, m, CH₂NH and CH₂CO), 2.50-2.60 (4H, m, CH₂CH₂NH), 2.65 (1H, m, CH₂NH), 2.75 (3H, s, CH₃), 2.85 (3H, s, CH₃), 3.10 (1H, m, CH₂NH), 3.90 (1H, m, CH₂NH), 4.45 (1H, m, CH₂NH), 4.60 (1H, m, CHN), 7.20-7.40 (5H, m, ArH), 9.80 (1H, m, ⁺NH(CH₃)₂).

MF C₂₁H₃₃N₃O₃, MW: 375.50

### Ex 104 4-(3-Hydroxycarbamoyl-propyl)-piperidine-1-carboxylic acid 2,4-dichloro-benzylamide

¹H NMR (DMSO-d₆, 400 MHz): δ (ppm) 0.87-1.03 (2H, m), 1.10-1.21 (2H, m), 1.30-1.45 (1H, m) 1.45-1.56 (2H, quin, J=7.5 Hz), 1.56-1.67 (2H, m), 1.92 (2H, m, CH₂CO), 2.66 (2H, m, N(CHH)₂), 3.97 (2H, m, N(CHH)₂), 4.23 (2H, s, PhCH₂), 7.08 (1H, s, NCONH), 7.27 (1H, d, J=8.0 Hz, ArH), 7.42 (1H, d, J=8.0 Hz, ArH), 7.56 (1H, s, ArH), 8.69 (1H, bs, OH), 10.35 (1H, bs, NHOH).

MF C₁₇H₂₃ClN₃O₃, MW 388.29

### Ex 105 4-[1-(2-(S)-Amino-4-phenyl-butyryl)-piperidin-4-yl]-N-hydroxy-butyramide

¹H NMR (DMSO-d₆, 400 MHz): δ (ppm) 0.80-1.20 (4H, m), 1.40-1.80 (5H, m), 1.90-2.00 (4H, m, CH₂CO and CH₂Ph), 2.55-2.60 (2H, m, CH₂CH₂), 2.65 (1H, m, CH₂NH), 2.95 (1H, q, J=14.8 Hz, CH₂NH), 3.60 (1H, m, CH₂NH), 4.20-4.30 (2H, m, CH₂NH and CHNH), 7.10-7.40 (5H, m, ArH), 8.20-8.45 (2H, bs, OH and NHCH), 10.36 (1H, s, NH).

MF C₁₉H₂₉N₃O₃, MW: 347.45

### Ex 106 4-(3-Hydroxycarbamoyl-propyl)-piperidine-1-carboxylic acid (furan-2-ylmethyl)-amide

¹H NMR (DMSO-d₆, 400 MHz): δ (ppm) 0.92 (2H, m), 1.07-1.22 (2H, m), 1.27-1.42 (1H, m) 1.49 (2H, m), 1.54-1.64 (2H, m), 1.91 (2H, t, J=7.2 Hz, CH₂CO), 2.60 (2H, m, N(CHH)₂), 3.93 (2H, m, N(CHH)₂), 4.18 (2H, s, NHCH₂), 6.09-6.16 (1H, m, FurH), 6.35 (1H, m, FurH), 6.90 (1H, m, NCONH), 7.52 (1H, m, FurH), 8.68 (1H, s, OH), 10.33 (1H, s, NHOH).

MF C₁₅H₂₃N₃O₄, MW 309.36

### Ex 107 Benzo[b]thiophene-2-carboxylic acid{4-amino-1-(R)[4-(3-hydroxycarbamoyl-propyl)-piperidine-1-carbonyl]-butyl}-amide

¹H NMR (DMSO-d₆, 400 MHz): δ (ppm) 0.70-1.20 (4H, m), 1.40-1.95 (11H, m, CH₂CH₂ and CH₂CO and CH₂N), 2.55 (1H, q, J=12.8 Hz, CH₂NH), 2.80 (2H, m, CH₂NH₂), 3.00 (1H, t, J=12.8 Hz, CH₂NH), 3.95 (1H, d, J=12.5 Hz, CH₂NH), 4.40 (1H, t, J=12.5 Hz, CH₂NH), 4.90 (1H, m, CHNH), 7.20-7.25 (2H, m, ArH), 7.65 (1H, m, NH⁺), 7.90-8.00 (2H, m, ArH), 8.20 (1H, s, ArH), 8.60 (1H, d, J=10.7 Hz, OH), 8.90-9.10 (1H, d, J=10.7 Hz, NHCH), 10.36 (1H, d, J=10.7 Hz, NH).

MF C₂₃H₃₂N₄O₄S, MW: 460.59

### Ex 108 4-[1-(3-(R)-Amino-4-naphthalen-2-yl-butyryl)-piperidin-4-yl]-N-hydroxy-butyramide

¹H NMR (DMSO-d₆, 400 MHz): δ (ppm) 0.78-1.18 (4H, m), 1.37-1.53 (3H, brs), 1.54-1.66 (2H, m), 1.91 (2H, dd, J=7.0 and 12.6 Hz), 2.52-2.68 (3H, m, N(CHH)₂, and CHHCHN), 2.89 (1H, m, N(CHH)₂), 3.02 (1H, m, CHHCHN), 3.09-3.15 (1H, m, CHHCHN), 3.64-3.70 (1H, m, N(CHH)₂), 3.79 (1H, brs, CH₂CHN), 4.34 (1H, d, J=12.7 Hz, N(CHH)₂), 7.42-7.54 (3H, m, ArH), 7.76-7.92 (7H, m, ⁺NH₃, ArH), 8.68 (1H, brs, OH), 10.36 (1H, d, J=10.7 Hz, NH).

MF C₂₃H₃₁N₃O₃ MW 379.50

### Ex 119 4-[1-(3-(S)-Amino-3-naphthalen-2-yl-propionyl)-piperidin-4-yl]-N-hydroxy-butyramide

¹H NMR (DMSO-d₆, 400 MHz): δ (ppm) 0.67-0.90 (2H, m), 0.97-1.12 (2H, m), 1.35-1.65 (5H, m), 1.85-1.92 (2H, m), 2.54 (1H, m), 2.92 (1H, q), 2.99-3.10 (2H, m, COCH₂CH), 3.77 (1H, d, J=13.9 Hz, N(CHH)₂), 4.37 (1H, d, J=12.6 Hz, N(CHH)₂), 4.78 (1H, s, CHNH₂), 7.56-7.58 (2H, m, ArH), 7.62 (1H, d, ArH), 7.90-7.98 (3H, m, ArH), 8.00 (1H, s, ArH), 8.35 (3H, s, NH₃⁺), 8.67 (1H, d, OH), 10.34 (1H, d, NH).

MF C₂₂H₂₉N₃O₃, MW 383.48

### Ex 110 Benzo[b]thiophene-2-carboxylic acid{3-hydroxy-1-(R)-[4-(3-hydroxycarbamoyl-propyl)-piperidine-1-carbonyl]-propyl}-amide

¹H NMR (DMSO-d₆, 400 MHz): δ (ppm) 0.70-1.20 (4H, m), 1.40-1.95 (9H, m, CH₂NH and CHCH₂CH₂ and CH₂CO), 2.55 (1H, m, CH₂NH), 3.00 (1H, t, J=12.8 Hz, CH₂NH), 3.40-3.50 (2H, m, CH₂OH), 4.00 (1H, t, J=12.5 Hz, CH₂NH), 4.35 (1H, d, J=12.5 Hz, CH₂NH), 5.00 (1H, m, CHNH), 7.30-7.45 (2H, m, ArH), 7.85-8.00 (2H, m, ArH), 8.20 (1H, s, ArH), 8.60-8.65 (1H, m, OH), 8.90-9.10 (1H, m, NHCH), 10.36 (1H, m, NHOH).

MF C₂₂H₂₉N₃O₅S, MW: 460.59

### Ex 111 4-[1-(3-(R)-Amino-3-naphthalen-2-yl-propionyl)-piperidin-4-yl]-N-hydroxy-butyramide

¹H NMR (DMSO-d₆, 400 MHz): 6?(ppm) 0.65-0.93 (2H, m), 0.97-1.07 (2H, m), 1.35-1.61 (5H, m), 1.84-1.92 (2H, m), 2.54 (1H, s, N(CHH)₂), 2.91 (1H, q, N(CHH)₂), 2.96-3.15 (2H, m, CH₂CH₂CO), 3.77 (1H, d, J=13.2 Hz, N(CHH)₂), 4.37 (1H, d, J=12.8 Hz, N(CHH)₂), 4.78 (1H, s, CHNH₂), 7.56-7.58 (2H, m, ArH), 7.90-7.98 (3H, m, ArH), 8.00 (1H, s), 8.38-8.45 (1H, d, OH), 10.34 (1H, s, NH).

MF C₂₂H₂₉NO₃, MW 383.48

### Ex 112 4-[1-(3-(S)-Amino-3-phenyl-propionyl)-piperidin-4-yl]-N-hydroxy-butyramide

¹H NMR (DMSO-d₆, 400 MHz): δ (ppm) 0.76-0.95 (2H, m), 1.10-1.17 (2H, m), 1.39-1.52 (3H, m), 1.56-1.69 (2H, m), 1.90 (2H, q, J=7.1 Hz), 2.52 (1H, m, N(CHH)₂), 2.88-2.99 (3H, m, CH₂CO and N(CHH)₂), 3.75 (1H, d, J=13.6 Hz, N(CHH)₂), 4.37 (1H, d, J=12.8 Hz, N(CHH)₂), 4.60 (1H, brs, CHNH₂), 7.32-7.50 (5H, m, ArH), 8.28 (1H, d, OH), 10.33 (1H, s, NH).

MF C₁₈H₂₇NO₃, MW 333.42

### Ex 113 Benzo[b]thiophene-2-carboxylic acid{3-hydroxy-1-(S)-[4-(3-hydroxycarbamoyl-propyl)-piperidine-1-carbonyl]-propyl}-amide

MF C₂₂H₂₉N₃O₅S, MW 460.59

### Ex 114 Benzo[b]thiophene-5-carboxylic acid{2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.70-1.20 (4H, m), 1.40-1.80 (5H, m), 1.95 (2H, q, J=7.4 Hz, CH₂CO), 2.55 (1H, t, J=11.9 Hz, CH₂NH), 3.05 (1H, t, J=12.5 Hz, CH₂NH), 3.55 (1H, m, CH₂OH), 3.75 (1H, m, CH₂OH), 4.10 (1H, d, J=11.9 Hz, CH₂NH), 4.40 (1H, d, J=11.9 Hz, CH₂NH), 5.00 (1H, m, CHCH₂OH), 7.55 (1H, d, J=5.4 Hz, ArH), 7.65 (2H, m, ArH), 8.10 (1H, d, J=7.2 Hz, ArH), 8.45 (1H, d, J=5.4 Hz, ArH), 8.50-8.60 (1H, m, NHCH), 8.80 (1H, m, NHCH), 10.35 (1H, s, NH).

MF C₂₁H₂₇N₃O₅S, MW: 433.52

### Ex 115 2-oxo-ethyl}-amideBenzo[b]thiophene-2-carboxylic acid{1-(R)-aminomethyl-2-[4-(3-hydroxycarbamoyl-propyl)-piperidin-1-yl]-

¹H NMR (DMSO-d₆, 400 MHz): δ (ppm) 0.75-1.25 (4H, m), 1.40-1.80 (5H, m), 1.85-1.95 (2H, dt, J=7.2 Hz, CH₂CO), 2.55 (1H, m, CH₂NH), 3.05 (1H, m, CH₂NH), 3.06-3.20 (2H, m, CH₂NH₂), 3.80 (1H, m, CH₂NH), 4.40 (1H, m, CH₂NH), 5.15 (1H, m, CHCH₂NH₂), 7.20-7.30 (2H, m, ArH), 7.80-8.05 (3H, m, ArH and NH⁺), 7.65 (2H, m, ArH), 8.20 (1H, s, ArH), 8.50-8.60 (1H, m, NHCH), 9.25-9.40 (1H, d, J=8.8 Hz, NHCH), 10.35 (1H, m, NH).

MF C₂₁H₂₈N₄O₄S, MW: 432.54

### Ex 116 4-{1-[2-(S)-(2-Benzo[b]thiophen-3-yl-acetylamino)-3-hydroxy-propionyl]-piperidin-4-yl}-N-hydroxy-butyramide

¹H NMR (DMSO-d₆, 400 MHz): δ (ppm) 0.70-1.25 (4H, m), 1.40-1.80 (5H, m), 1.85-1.95 (2H, q, J=7.4 Hz, CH₂CO), 2.55 (1H, t, J=11.6 Hz, CH₂NH), 3.05 (1H, t, J=11.6 Hz, CH₂NH), 3.30-3.40 (2H, m, CH₂OH), 4.00 (1H, t, J=11.6 Hz, CH₂NH), 4.35 (1H, d, J=11.6 Hz, CH₂NH), 4.60 (1H, m, OH), 5.00 (1H, m, CHCH₂OH), 7.00-7.20 (2H, m, ArH), 7.85-8.05 (2H, m, ArH), 8.22 (1H, m, ArH), 8.65 (1H, s, OH), 8.85-8.95 (1H, m, NHCH), 10.35 (1H, m, NH).

MF C₂₂H₂₉N₃O₅S, MW: 447.55

### Ex 117 Benzo[b]thiophene-3-carboxylic acid{2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.70-1.30 (4H, m), 1.40-1.80 (5H, m), 1.85-1.95 (2H, m, CH₂CO), 2.55 (1H, t, J=11.8 Hz, CH₂NH), 3.05 (1H, t, J=11.8 Hz, CH₂NH), 3.50-3.75 (2H, m, CH₂OH), 4.10 (1H, d, J=11.6 Hz, CH₂NH), 4.40 (1H, d, J=11.8 Hz, CH₂NH), 4.85-5.00 (1H, dt, J=5.9 Hz, CHNH), 5.10 (1H, m, OH), 7.40-7.50 (2H, m, ArH), 8.04-8.10 (1H, d, J=7.9 Hz, ArH), 8.45-8.55 (2H, m, ArH), 8.60 (1H, d, J=7.9 Hz, NHCH), 8.75. 8-80 (1H, d, J=7.9 Hz, OH), 10.36 (1H, d, J=9.6 Hz, NH).

MF C₂₁H₂₇N₃O₅S, MW: 433.52

### Ex 118 Benzofuran-2-carboxylic acid {2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.65-1.30 (4H, m), 1.40-1.80 (5H, m), 1.85-1.95 (2H, m, CH₂CO), 2.55 (1H, t, J=11.8 Hz, CH₂NH), 3.05 (1H, t, J=11.8 Hz, CH₂NH), 3.50-3.75 (2H, m, CH₂OH), 4.10 (1H, d, J=11.6 Hz, CH₂NH), 4.40 (1H, d, J=11.8 Hz, CH₂NH), 5.10 (2H, m, OH and CHNH), 7.25-7.35 (2H, m, ArH), 7.60 (1H, d, J=8.40 Hz, ArH), 7.70 (1H, d, J=8.40 Hz, ArH), 7.80 (1H, d, J=8.40 Hz, ArH), 8.40-8.55 (2H, d, J=7.90 Hz, NHCH), 8.75. 8-80 (1H, m, OH), 10.36 (1H, s, NH).

MF C₂₁H₂₇N₃O₆, MW: 417.46

### Ex 119 N-Hydroxy-4-{1-[3-hydroxy-2-(S)-(2-naphthalen-1-yl-acetylamino)-propionyl]-piperidin-4-yl}-butyramide

¹H NMR (DMSO-d₆, 400 MHz): δ (ppm) 0.54-0.83 (1H, 2q), 0.84-1.17 (4H, m), 1.28-1.66 (5H, m), 1.85-1.95 (2H, m), 2.45 (1H, m, N(CHH)₂), 2.85 (1H, m, N(CHH)₂), 3.40 (1H, m, CHHO), 3.59 (1H, m, CHHO), 3.81-3.91 (1H, m, N(CHH)₂), 3.94 (2H, s, CH₂Naf), 4.35 (1H, m, N(CHH)₂), 4.79 (1H, m, CHNH₃⁺), 4.89 (1H, t, J=6.0 Hz, OH), 7.38-7.54 (4H, m, ArH), 7.76-7.93 (2H, m, ArH), 8.02-8.13 (1H, m, ArH), 8.36-8.49 (1H, 2d, J=8.22 Hz, NHCH), 8.68 (1H, brs, OH), 10.36 (1H, brs, NH).

MF C₂₄H₃₁N₃O₅, MW 441.52

### Ex 120 1H-Indole-2-carboxylic acid {2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.75-1.30 (4H, m), 1.40-1.80 (5H, m), 1.85-1.95 (2H, m, CH₂CO), 2.55 (1H, t, J=11.9 Hz, CH₂NH), 3.05 (1H, t, J=11.9 Hz, CH₂NH), 3.50-3.75 (2H, m, CH₂OH), 4.10 (1H, d, J=11.9 Hz, CH₂NH), 4.40 (1H, d, J=11.9 Hz, CH₂NH), 4.95 (1H, m, OH), 5.10 (1H, m, CHNH), 7.00 (1H, t, J=7.9 Hz, ArH), 7.15 (1H, t, J=7.90 Hz, ArH), 7.25 (1H, d, J=7.90 Hz, ArH), 7.40 (1H, d, J=7.9 Hz, ArH), 7.6 (1H, d, J=7.9 Hz, ArH), 8.04-8.10 (1H, d, J=7.9 Hz, ArH), 8.40-8.55 (1H, d, J=8.2 Hz, NHCH), 8.65 (1H, d, J=6.8 Hz, OH), 10.36 (1H, d, J=10.25 Hz, NH), 11.60 (1H, m, NHInd).

MF C₂₁H₂₈N₃O₅, MW: 416.47

### Ex 121 Quinoline-2-carboxylic acid {2-[4-(3-hydroxycarbamoyl-propyl)-piperidin-1-yl]-1-(S)-hydroxymethyl-2-oxo-ethyl}-amide

¹H NMR (DMSO-d⁶, 400 MHz): δ (ppm) 0.80-1.30 (4H, m), 1.45-1.80 (5H, m), 1.85-1.95 (2H, m, CH₂CO), 2.60 (1H, q, J=11.7 Hz, CH₂NH), 3.05 (1H, m, CH₂NH), 3.40 (2H, s, CH₂OH), 4.10 (1H, d, J=11.7 Hz, CH₂NH), 4.40 (1H, d, J=11.7 Hz, CH₂NH), 5.05 (1H, q, J=5.54 Hz, CHNH), 7.70 (1H, t, J=7.8 Hz, ArH), 7.90 (1H, t, J=7.8 Hz, ArH), 8.10-8.40 (3H, m, ArH), 8.60 (1H, d, J=8.6 Hz, ArH), 8.80-8.90 (1H, m, NHCH), 10.36 (1H, d, J=7.8 Hz, NHOH).

MF C₂₂H₂₈N₄O₅, MW: 428.48

### Ex 122 Isoquinoline-1-carboxylic acid {2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.75-1.30 (4H, m), 1.45-1.80 (5H, m), 1.85-1.95 (2H, q, J=7.6 Hz CH₂CO), 2.65 (1H, m, CH₂NH), 3.05 (1H, t, J=12.0 Hz, CH₂NH), 3.55-3.65 (2H, m, CH₂OH), 4.10 (1H, d, J=12.0 Hz, CH₂NH), 4.40 (1H, d, J=12.0 Hz, CH₂NH), 5.05-5.10 (1H, m, CHNH), 7.40 (1H, t, J=7.8 Hz, ArH), 7.75 (1H, t, J=7.8 Hz, ArH), 8.05 (2H, d, J=6.6 Hz, ArH), 8.58 (1H, d, J=5.6 Hz, ArH), 9.15 (1H, d, J=8.4 Hz, ArH), 8.85-8.95 (1H, m, NHCH), 10.35 (1H, m, NHOH).

MF C₂₂H₂₈N₄O₅, MW 428.48

### Ex 123 Isoquinoline-3-carboxylic acid {2-[4-(3-hydroxycarbamoyl-propyl)-piperidin-1-yl]-1-(S)-hydroxymethyl-2-oxo-ethyl}-amide

¹H NMR (DMSO-d₆, 400 MHz): δ (ppm) 0.75-1.30 (4H, m), 1.45-1.80 (5H, m), 1.80-1.95 (2H, q, J=7.8 Hz CH₂CO), 2.65 (1H, q, J=11.9 Hz, CH₂NH), 3.05 (1H, m, CH₂NH), 3.55-3.65 (2H, m, CH₂OH), 4.10 (1H, d, J=12.6 Hz, CH₂NH), 4.40 (1H, d, J=12.6 Hz, CH₂NH), 5.05-5.10 (1H, m, CHNH), 7.80-7-92 (2H, dt, J=7.9 Hz, ArH), 8.22 (1H, d, J=8.2 Hz, ArH), 8.28 (1H, d, J=8.2 Hz, ArH), 8.58 (1H, d, J=4.6 Hz, ArH), 8.85-8.95 (1H, m, NHCH), 9.40 (1H, s, ArH), 10.35 (1H, m, NHOH).

MF C₂₂H₂₈N₄O₅, MW 428.48

### Ex 124 Benzo[b]thiophene-2-carboxylic acid{1-aminomethyl-2-[4-(3-hydroxycarbamoyl-propyl)-piperidin-1-yl]-2-oxo-ethyl}-amide

¹H NMR (DMSO-d₆, 400 MHz): δ (ppm) 0.80-1.30 (4H, m), 1.45-1.80 (5H, m), 1.80-1.95 (2H, dt, J=7.2 Hz, CH₂CO), 2.65 (1H, m, CH₂NH), 3.05 (1H, m, CH₂NH), 3.00-3.20 (2H, m, CH₂NH₂), 4.10 (1H, m, CH₂NH), 4.40 (1H, m, CH₂NH), 5.05-5.10 (1H, m, CHNH), 7.40-7.50 (2H, m, ArH), 7.80-8.10 (3H, m, NH₃⁺, ArH), 8.20 (1H, s, ArH), 8.60 (1H, bs, OH), 9.30-9.40 (1H, m, NHCH), 10.35 (1H, m, NHOH).

MF C₂₂H₂₈N₄O₅, MW: 432.54

### Ex 125 4-(3-Hydroxycarbamoyl-propyl)-piperidine-1-carboxylic acid 4-chloro-2-fluoro-benzylamide

¹H NMR (DMSO-d₆, 400 MHz): δ (ppm) 0.84-1.02 (2H, m), 1.10-1.26 (2H, m), 1.29-1.43 (1H, m) 1.43-1.70 (4H, m), 1.92 (2H, t, J=7.2 Hz, CH₂CO), 2.63 (2H, m, N(CHH)₂), 3.94 (2H, m, N(CHH)₂), 4.21 (2H, s, PhCH₂), 7.02 (1H, s, NCONH), 7.18-7.42 (3H, m, ArH), 8.67 (1H, bs, OH), 10.33 (1H, s, NHOH).

MF C₁₇H₂₃ClFN₃O₃, MW 371.83

### Ex 126 4-(3-Hydroxycarbamoyl-propyl)-piperidine-1-carboxylic acid (2-amino-4-methyl-phenyl)-amide

¹H NMR (DMSO-d₆, 400 MHz): δ (ppm) 1.05 (2H, dq, J=3.5 and 12.6 Hz), 1.14-1.28 (2H, m), 1.38-1.48 (1H, m) 1.53 (2H, quin, J=7.4 Hz), 1.62-1.73 (2H, m), 1.95 (2H, t, J=7.2 Hz, CH₂CO), 2.25 (3H, s, CH₃), 2.77 (2H, m, N(CHH)₂), 4.10 (2H, m, N(CHH)₂), 6.76-6.93 (2H, m, ArH), 7.23 (1H, m, ArH), 8.29 (3H, bs, NH₂⁺) 8.71 (1H, bs, OH), 10.36 (1H, s, NHOH).

MF C₁₇H₂₆N₄O₃, MW 334.41

### Ex 127 4-(3-Hydroxycarbamoyl-propyl)-piperidine-1-carboxylic acid (2-fluoro-6-methoxy-phenyl)-amide

¹H NMR (DMSO-d₆, 400 MHz): δ (ppm) 1.02 (2H, dq, J=3.5 and 12.6 Hz), 1.14-1.27 (2H, m), 1.33-1.48 (1H, m) 1.53 (2H, m), 1.59-1.70 (2H, m), 1.95 (2H, t, J=7.2 Hz, CH₂CO), 2.72 (2H, m, N(CHH)₂), 3.78 (3H, s, OCH₃), 4.06 (2H, m, N(CHH)₂), 6.79 (1H, m, ArH), 6.82-6.88 (1H, m, ArH), 7.14. 7.23 (1H, m, ArH), 7.68 (1H, s, NCONH), 8.70 (1H, bs, OH), 10.36 (1H, s, NHOH).

MF C₁₇H₂₄FN₃O₄, MW 353.39

### Ex 128 4-(3-Hydroxycarbamoyl-propyl)-piperidine-1-carboxylic acid (2-fluoro-5-methyl-phenyl)-amide

¹H NMR (DMSO-d₆, 400 MHz): δ (ppm) 1.02 (2H, dq, J=3.8 and 12.6 Hz), 1.13-1.28 (2H, m), 1.34-1.47 (1H, m) 1.53 (2H, m), 1.60-1.72 (2H, m), 1.94 (2H, t, J=7.2 Hz, CH₂CO), 2.25 (3H, s, CH₃), 2.74 (2H, m, N(CHH)₂), 4.06 (2H, m, N(CHH)₂), 6.85-6.92 (1H, m, ArH), 6.98-7.08 (1H, m, ArH), 7.18-7.25 (1H, m, ArH), 8.14 (1H, s, NCONH), 8.69 (1H, s, OH), 10.36 (1H, s, NHOH).

MF C₁₇H₂₄FN₃O₃, MW 337.39

### Ex 129 4-(3-Hydroxycarbamoyl-propyl)-piperidine-1-carboxylic acid (2-chloro-6-fluoro-phenyl)-amide

¹H NMR (DMSO-d₆, 400 MHz): δ (ppm) 0.93-1.14 (2H, m), 1.14-1.32 (2H, m), 1.38-1.60 (3H, m) 1.60-1.75 (2H, m), 1.95 (2H, t, J=7.2 Hz, CH₂CO), 2.78 (2H, m, N(CHH)₂), 4.07 (2H, m, N(CHH)₂), 7.13-7.42 (3H, m, ArH), 8.21 (1H, s, NCONH), 8.70 (1H, bs, OH), 10.36 (1H, s, NHOH).

MF C₁₆H₂₁ClFN₃O₃, MW 357.81

### Ex 130 4-[1-(3-(R)-Amino-3-phenyl-propionyl)-piperidin-4-yl]-N-hydroxy-butyramide

¹H NMR (DMSO-d₆, 400 MHz): 6 ?(ppm) 0.76-0.92 (2H, m), 1.07-1.28 (2H, m), 1.39-1.64 (5H, m), 1.92 (2H, q, J=7.1 Hz), 2.52 (1H, s, N(CHH)₂), 2.86-3.01 (3H, m, CH₂CO, N(CHH)₂), 3.75 (1H, d, J=13.1 Hz, N(CHH)₂), 4.37 (1H, d, J=12.6 Hz, N(CHH)₂), 4.60 (1H, s, CHNH₂), 7.36-7.49 (5H, m, ArH), 8.23 (3H, bs, NH₃⁺), 8.67 (1H, s, OH), 10.34 (1H, s, NH).

MF C₁₈H₂₇NO₃, MW 333.42

### Ex 131 4-[1-(3-(S)-Amino-5-phenyl-pentanoyl)-piperidin-4-yl]-N-hydroxy-butyramide

¹H NMR (DMSO-d₆, 400 MHz): δ (ppm) 0.89-1.03 (2H, m), 1.16 (2H, m), 1.45-1.53 (2H, m), 1.66-1.95 (5H, m), 2.59 (2H, m, N(CHH)₂), 2.68 (2H, t, J=8.0 Hz), 2.83 (1H, dd), 2.97 (1H, t, J=12.3 Hz, CH₂CO), 3.82 (1H, d, J=13.6 Hz, N(CHH)₂), 4.41 (1H, bs, CHNH₂), 7.21-7.32 (5H, m, ArH), 7.79 (3H, bs, NH₃⁺), 8.70 (1H, s, OH), 10.36 (1H, s, NH).

MF C₂₀H₃₁NO₃, MW 361.48

### Ex 132 4-(3-Hydroxycarbamoyl-propyl)-piperidine-1-carboxylic acid (4-butyl-phenyl)-amide

¹H NMR (CDCl₃-d₁, 400 MHz): δ (ppm) 1.37-1.58 (4H, m), 1.65-1.81 (1H, m), 1.81-2.09 (4H, m), 2.39 (2H, m, CH₂CO), 3.18 (2H, m, N(CHH)₂), 4.41-4.58 (2H, m, N(CHH)₂), 7.60-7.72 (2H, m, ArH), 7.72-7.82 (1H, m, ArH), 8.42 (1H, d, J=8.0 Hz, ArH), 8.74 (1H, d, J=8.0 Hz, ArH), 9.02 (1H, s, ArH), 9.62 (1H, s, NCONH).

MF C₁₉H₂₄N₄O₃, MW 356.42

### Ex 133 N-Hydroxy-4-(1-phenylsulfamoyl-piperidin-4-yl)-butyramide

¹H NMR (DMSO-d₆, 400 MHz): 6?(ppm) 0.85-0.95 (2H, m), 1.04-1.11 (2H, m), 1.17-1.25 (1H, brs), 1.37-1.47 (2H, m), 1.57-1.65 (2H, d, J=12.4 Hz), 1.88 (2H, t, J=7.2 Hz), 2.61 (2H, t, J=11.8 Hz), 3.57 (2H, d, 12.1 Hz), 7.01-7.04 (1H, t, ArH, J=7.3 Hz), 7.16-7.18 (2H, d, ArH, J=8.3 Hz), 7.26-7.30 (2H, t, ArH, J=7.7 Hz), 8.70 (1H, s, OH), 9.85 (1H, s, NHPh), 10.30 (1H, s, NH).

MF C₁₅H₂₃N₃O₄S, MW 341.43

### Ex 134 4-[1-(3-(R)-Amino-5-phenyl-pentanoyl)-piperidin-4-yl]-N-hydroxy-butyramide

¹H NMR (DMSO-d₆, 400 MHz): 6?(ppm) 1.05-1.21 (2H, m), 1.37 (2H, m), 1.69 (3H, m), 1.87 (2H, brs), 2.13 (2H, s), 2.45 (2H, m, CH₂CO, N(CHH)₂), 3.00-3.20 (3H, m, CH₂Ph, CH₂CO), 3.86 (2H, s, NCH), 4.59 (1H, d, J=12.7 Hz, N(CHH)₂), 7.47-7.58 (5H, m, ArH), 8.04-8.06 (3H, bs, NH₃⁺), 8.86 (1H, s, OH), 10.56 (1H, s, NH).

MF C₁₉H₂₉N₃O₃, MW 347.45

### Ex 135 4-(3-Hydroxycarbamoyl-propyl)-piperidine-1-carboxylic acid biphenyl-4-ylamide

¹H NMR (DMSO-d₆, 400 MHz): δ (ppm) 0.93-1.13 (2H, m), 1.13-1.28 (2H, m), 1.33-1.47 (1H, m), 1.52 (2H, m), 1.61-1.75 (2H, m), 1.94 (2H, t, J=7.2 Hz, CH₂CO), 2.75 (2H, m, N(CHH)₂), 4.12 (2H, m, N(CHH)₂), 7.30 (1H, m, ArH), 7.38-7.46 (2H, m, ArH), 7.50-7.59 (4H, m, ArH), 7.62 (2H, d, J=7.6 Hz, ArH), 8.55 (1H, s, NCONH), 8.70 (1H, bs, OH), 10.34 (1H, s, NHOH).

MF C₂₂H₂₇N₃O₃, MW 381.47

### Ex 136 4-(3-Hydroxycarbamoyl-propyl)-piperidine-1-carboxylic acid (benzo[1,3]dioxol-5-ylmethyl)-amide

¹H NMR (DMSO-d₆, 400 MHz): δ (ppm) 0.83-1.00 (2H, m), 1.08-1.22 (2H, m), 1.27-1.41 (1H, m), 1.49 (2H, m), 1.54-1.65 (2H, m), 1.92 (2H, t, J=7.2 Hz, CH₂CO), 2.61 (2H, m, N(CHH)₂), 3.94 (2H, m, N(CHH)₂), 4.10 (2H, s, OCH₂O), 5.96 (2H, s, CH₂Ph), 6.66-6.73 (1H, m, ArH), 6.77-6.85 (2H, m, ArH), 6.93 (1H, m, NCONH), 10.33 (1H, s, NHOH).

MF C₁₈H₂₅N₃O₅, MW 363.41

### Ex 137 4-(3-Hydroxycarbamoyl-propyl)-piperidine-1-carboxylic acid (4-propoxy-phenyl)-amide

¹H NMR (DMSO-d₆, 400 MHz): δ (ppm) 0.93-1.07 (2H, m), 1.13-1.24 (2H, m), 1.32-1.46 (1H, m), 1.52 (2H, m), 1.59-1.72 (2H, m), 1.93 (2H, t, J=7.2 Hz, CH₂CO), 2.73 (2H, m, N(CHH)₂), 4.09 (2H, m, N(CHH)₂), 6.92 (4H, m, ArH), 7.07 (1H, m), 7.34 (2H, m, ArH), 7.46 (2H, d, J=8.4 Hz, ArH), 8.46 (1H, s, NCONH), 8.68 (1H, s, OH), 10.34 (1H, s, NHOH).

MF C₂₂H₂₇N₃O₄, MW 397.47

### Ex 138 4-(3-Hydroxycarbamoyl-propyl)-piperidine-1-carboxylic acid (4-propoxy-phenyl)-amide

¹H NMR (DMSO-d₆, 400 MHz): δ (ppm) 0.90-1.04 (2H, m), 0.95 (3H, t, J=7.2 Hz, CH₃), 1.13-1.26 (2H, m), 1.33-1.45 (1H, m), 1.51 (2H, quin, J=7.4 Hz), 1.59-1.75 (4H, m), 1.93 (2H, t, J=7.2 Hz, CH₂CO), 2.70 (2H, m, N(CHH)₂), 3.84 (2H, t, J=6.4 Hz, PhOCH₂), 4.07 (2H, m, N(CHH)₂), 6.78 (2H, d, J=8.8 Hz, ArH), 7.30 (2H, d, J=8.8 Hz, ArH), 8.25 (1H, s, NCONH), 8.68 (1H, bs, OH), 10.34 (1H, s, NHOH).

MF C₁₉H₂₉N₃O₄, MW 397.47

### Ex 139 4-(3-Hydroxycarbamoyl-propyl)-piperidine-1-carboxylic acid (4-isopropoxy-phenyl)-amide

¹H NMR (DMSO-d₆, 400 MHz): δ (ppm) 0.90-1.06 (2H, m), 1.12-1.20 (2H, m), 1.22 (6H, d, J=6.0 Hz, CH(CH₃)₂), 1.32-1.45 (1H, m), 1.51 (2H, m), 1.59-1.69 (2H, m), 1.93 (2H, t, J=7.2 Hz, CH₂CO), 2.70 (2H, m, N(CHH)₂), 4.07 (2H, m, N(CHH)₂), 4.48 (1H, hept, J=6.0 Hz, CH(CH₃)₂), 6.77 (2H, d, J=8.4 Hz, ArH), 7.29 (2H, d, J=8.4 Hz, ArH), 8.25 (1H, s, NCONH), 8.39-8.93 (1H, bs, OH), 10.34 (1H, s, NHOH).

MF C₂₀H₂₉N₃O₄, MW 363.45

### Ex 140 4-(3-Hydroxycarbamoyl-propyl)-piperidine-1-carboxylic acid (1-(R)-phenyl-ethyl)-amide

¹H NMR (DMSO-d₆, 400 MHz): δ (ppm) 0.83-0.99 (2H, m), 1.09-1.20 (2H, m), 1.26-1.40 (1H, m), 1.33 (3H, d, J=6.8 Hz, CH₃), 1.43-1.53 (2H, m), 1.54-1.64 (2H, m), 1.92 (2H, t, J=7.2 Hz, CH₂CO), 2.59 (2H, m, N(CHH)₂), 3.98 (2H, m, N(CHH)₂), 4.81 (1H, m, CHNH), 6.69 (1H, d, J=8.0 Hz, ArH), 7.14-7.21 (1H, m, CHNH), 7.26-7.32 (4H, m, ArH), 8.68 (1H, s, OH), 10.33 (1H, s, NHOH).

MF C₁₈H₂₇N₃O₃, MW 333.42

### Ex 141 4-(3-Hydroxycarbamoyl-propyl)-piperidine-1-carboxylic acid (1-(S)-phenyl-ethyl)-amide

MF C₁₈H₂₇NO₃, MW 333.42

### Ex 142 4-[1-(3-(R)-Amino-3-naphthalen-2-yl-propionyl)-piperidin-4-yl]-N-hydroxy-butyramide

MF C₂₂H₂₉N₃O₃, MW 383.48

### Ex 143 4-(3-Hydroxycarbamoyl-propyl)-piperidine-1-carboxylic acid quinolin-2-ylamide

¹H NMR (DMSO-d₆, 400 MHz): δ (ppm) 1.01-1.15 (2H, m), 1.16-1.25 (2H, m), 1.40-1.60 (3H, m), 1.66-1.78 (2H, m), 1.94 (2H, t, J=7.2 Hz, CH₂CO), 2.88 (2H, m, N(CHH)₂), 4.20 (2H, m, N(CHH)₂), 7.56 (1H, m, ArH), 7.75-7.87 (2H, m, ArH), 7.75-7.87 (2H, m, ArH), 7.98 (2H, d, J=7.6 Hz, ArH), 8.48 (1H, m, ArH), 10.34 (1H, s, NHOH).

MF C₁₉H₂₄N₄O₃, MW 356.42

### Ex 144 4-[1-(3-(R)-Amino-4-naphthalen-2-yl-butyryl)-piperidin-4-yl]-N-hydroxy-butyramide

¹H NMR (DMSO-d₆, 400 MHz): δ (ppm) 0.70-1.30 (4H, m), 1.45-1.80 (5H, m), 1.80-1.95 (2H, m, CH₂CO), 2.65 (2H, t, J=6.6 Hz, COCH₂CH), 2.70-2.85 (1H, q, J=6.6 Hz, CH₂NH), 3.00-3.20 (2H, m, CH₂Ph), 3.30-3.80 (3H, m, CH₂NH, and CHNH), 4.35 (1H, m, CHNH), 7.40-7.55 (3H, m, ArH), 7.80 (1H, s, ArH), 7.85-7.95 (3H, m, ArH), 8.00-8.20 (1H, m, NH₃⁺), 8.36 (1H, s, OH), 10.35 (1H, s, NHOH).

MF C₂₃H₃₁N₃O₅, MW 397.51

### Ex 145 4-(3-Hydroxycarbamoyl-propyl)-piperidine-1-carboxylic acid [4-(2-diethylamino-ethyl)-phenyl]-amide

¹H NMR (DMSO-d₆, 400 MHz): δ (ppm) 0.99 (2H, m), 1.13-1.24 (2H, m), 1.21 (6H, t, J=7.3, N(CH₂CH₃)₂), 1.35-1.46 (1H, m), 1.48-1.57 (2H, m), 1.61-1.70 (2H, m), 1.93 (2H, t, J=7.3 Hz, CH₂CO), 2.72 (2H, m, N(CHH)₂), 2.82-2.91 (2H, m, PhCH₂), 3.16-3.27 (6H, m, CH₂N(CH₂ CH₃)₂), 4.08 (2H, m, N(CHH)₂), 7.15 (2H, d, J=8.6 Hz, ArH), 7.41 (2H, d, J=8.6 Hz, ArH), 8.41 (1H, m, NHCON), 9.11 (1H, bs, OH), 10.32 (1H, s, NHOH).

MF C₂₂H₃₆N₄O₃, MW 404.55

### Ex 146 4-[1-(3-(S)-Amino-4-benzo[b]thiophen-3-yl-butyryl)-piperidin-4-yl]-N-hydroxy-butyramide

¹H NMR (DMSO-d₆, 400 MHz): δ (ppm) 0.82-0.94 (2H, m), 1.12-1.17 (2H, m), 1.39-1.69 (5H, m), 1.92 (2H, m), 2.54-2.75 (3H, m, COCH₂CH, N(CHH)₂), 2.87-2.95 (1H, m, N(CHH)₂), 3.16 (2H, m, NCHCHH), 3.66-3.77 (2H, m, NCHCH₂, N(CHH)₂), 4.38 (1H, d, J=13.6 Hz, N(CHH)₂), 7.40-7.47 (2H, m, ArH), 7.61 (1H, s, ArH), 7.91-8.04 (3H, m, ArH and NH⁺), 8.66 (1H, brs, OH), 10.31 (1H, s, NHOH).

MF C₂₁H₂₉N₃O₅S, MW: 403.54

### Ex 147 4-[1-(3-(R)-Amino-4-benzo[b]thiophen-3-yl-butyryl)-piperidin-4-yl]-N-hydroxy-butyramide

MF C₂₁H₂₉N₃O₅S, MW: 403.54

### Ex 148 Benzo[b]thiophene-2-carboxylic acid{1-dimethylaminomethyl-2-[4-(3-hydroxycarbamoyl-propyl)-piperidin-1-yl]-2-oxo-ethyl}-amide

¹H NMR (DMSO-d₆, 400 MHz): δ (ppm) 0.80-1.30 (4H, m), 1.45-1.80 (5H, m), 1.85-1.95 (2H, dt, J=7.23 Hz, CH₂CO), 2.50-2.70 (1H, m, CH₂NH), 2.90-3.10 (1H, q, J=12.0 Hz, CH₂NH), 3.00-3.20 (2H, m, CH₂Ph), 3.80 (1H, t, J=12.0 Hz, CH₂NH), 4.35 (1H, d, J=11.7 Hz, CH₂NH), 5.30 (1H, m, CH₂CHNH), 7.40-7.50 (2H, m, ArH), 7.95 (1H, t, J=8.1 Hz, ArH), 8.05 (1H, t, J=7.6 Hz, ArH), 8.20 (1H, s, ArH), 8.60 (1H, bs, OH), 9.35-9.45 (1H, d, J=8.8 Hz, NHCH), 10.35 (1H, m, NHOH).

MF C₂₃H₃₂N₄O₄S, MW: 460.59

### Ex 149 4-[1-(3-(S)-Amino-3-naphthalen-2-yl-propionyl)-piperidin-4-yl]-N-hydroxy-butyramide

¹H NMR (DMSO-d₆, 400 MHz): δ (ppm) 0.63-1.12 (4H, m), 1.44 (3H, m), 1.60 (2H, m), 1.89-1.91 (2H, td, J=7.3 and 14.6 Hz), 2.64 (3H, s, CH₃), 2.79 (3H, s, CH₃), 2.98 (1H, q, J=10.7 Hz), 3.41 (3H, m, NCHCH₂ and N(CHH)₂), 3.96 (1H, d, J=12.8 Hz, N(CHH)₂), 4.28 (1H, d, J=11.6 Hz, N(CHH)₂), 5.01 (1H, s, NCHCH₂), 7.59-8.12 (7H, m, ArH), 9.72 (1H, s, OH), 10.29 (1H, s, NH).

MF C₂₄H₃₃N₃O₃, MW 411.54

### Ex 150 4-(3-Hydroxycarbamoyl-propyl)-piperidine-1-carboxylic acid biphenyl-3-ylamide

MF C₂₂H₂₇N₃O₃, MW 381.46

### Ex 151 4-(3-Hydroxycarbamoyl-propyl)-piperidine-1-carboxylic acid (4-pyridin-2-yl-phenyl)-amide

¹H NMR (DMSO-d₆, 400 MHz): δ (ppm) 1.03 (2H, dq, J=3.2 and 12.0 Hz), 1.11-1.28 (2H, m), 1.36-1.48 (1H, m), 1.53 (2H, quin, J=7.4 Hz), 1.62-1.72 (2H, m), 1.94 (2H, t, J=7.3 Hz, CH₂CO), 2.77 (2H, m, N(CHH)₂), 4.13 (2H, m, N(CHH)₂), 7.41-7.53 (2H, m, ArH), 7.65 (2H, d, J=8.0 Hz, ArH), 7.95 (2H, d, J=8.0 Hz, ArH), 8.00-8.13 (2H, m, ArH), 8.67 (1H, m, NHCON), 8.72 (1H, bs, OH), 10.32 (1H, s, NHOH).

MF C₂₁H₂₆N₄O₃, MW 382.46

### Ex 152 4-(3-Hydroxycarbamoyl-propyl)-piperidine-1-carboxylic acid (4-oxazol-5-yl-phenyl)-amide

¹H NMR (DMSO-d₆, 400 MHz): δ (ppm) 1.02 (2H, dq, J=3.0 and 12.4 Hz), 1.13-1.24 (2H, m), 1.34-1.46 (1H, m), 1.52 (2H, quin, J=7.4 Hz), 1.62-1.73 (2H, m), 1.94 (2H, t, J=7.3 Hz, CH₂CO), 2.75 (2H, m, N(CHH)₂), 4.11 (2H, m, N(CHH)₂), 7.51 (1H, s, OxazH), 7.56-7.60 (4H, m, ArH), 8.35 (1H, s, OxazH), 8.62 (1H, m, NHCON), 8.65 (1H, s, OH), 10.32 (1H, s, NHOH).

MF C₁₉H₂₄N₄O₄, MW 372.42

### Ex 153 4-(3-Hydroxycarbamoyl-propyl)-piperidine-1-carboxylic acid (4-pyridin-3-yl-phenyl)-amide

MF C₂₁H₂₆N₄O₃, MW 382.45

### Ex 154 4-[1-(3-(S)-Amino-4-naphthalen-1-yl-butyryl)-piperidin-4-yl]-N-hydroxy-butyramide

MF C₂₃H₃₁N₃O₃, MW 397.51

### Ex 155 4-[1-(3-(R)-Amino-4-naphthalen-1-yl-butyryl)-piperidin-4-yl]-N-hydroxy-butyramide

¹H NMR (DMSO-d₆, 400 MHz): δ (ppm) 0.80-0.95 (2H, m), 1.09-1.20 (2H, m), 1.39-1.70 (5H, m), 1.92 (2H, q, J=7.4 Hz), 2.62-2.67 (2H, m, CH₂CHNH₂CH₂), 2.89 (1H, q, J=12.1 Hz), 3.37 (2H, m, CH₂CHNH₂CH₂), 3.67 (1H, d, J=13.9 Hz, N(CHH)₂), 3.77 (1H, s, CH₂CHNH₂CH₂), 4.35 (1H, d, J=14.2 Hz, N(CHH)₂, 7.42-7.63 (4H, m, ArH), 7.87-7.98 (3H, m, ArH and NH⁺), 8.21 (1H, d, J=8.3 Hz, ArH), 8.60 (1H, bs, OH), 10.32 (1H, s, NHOH).

MF C₂₃H₃₁N₃O₃, MW 397.51

### Ex 156 4-(4-(hydroxyamino)-4-oxobutyl)-N-((2-phenylthiazol-4-yl)methyl)piperidine-1-carboxamide

¹H NMR (DMSO-d₆, 400 MHz): δ (ppm) 0.96 (2H, m), 1.11-1.21 (2H, m), 1.31-1.45 (1H, m) 1.50 (2H, quint, J=7.6 Hz), 1.56-1.65 (2H, m), 1.92 (2H, t, J=7.6 Hz, CH₂CO), 2.65 (2H, m, N(CHH)₂), 3.98 (2H, m, N(CHH)₂), 4.36 (2H, m, PhCH₂), 7.04 (1H, bs, NCONH), 7.29 (1H, s, ThiazH), 7.33-7.53 (3H, m, ArH), 7.87-7.95 (2H, m, ArH), 10.30 (1H, s, NHOH).

MF C₂₀H₂₆N₄O₃S, MW 402.51

### Ex 157 N-(benzo[b]thiophen-3-ylmethyl)-4-(4-(hydroxyamino)-4-oxobutyl)piperidine-1-carboxamide

¹H NMR (DMSO-d₆, 400 MHz): δ (ppm) 0.93 (2H, dq, J=3.6 and 12.4 Hz), 1.10-1.20 (2H, m), 1.30-1.43 (1H, m) 1.50 (2H, quint, J=7.6 Hz), 1.54-1.63 (2H, m), 1.92 (2H, t, J=7.6 Hz, CH₂CO), 2.63 (2H, m, N(CHH)₂), 3.97 (2H, m, N(CHH)₂), 4.46 (2H, m, PhCH₂), 6.96 (1H, bs, NCONH), 7.33-7.43 (3H, m, ArH), 7.93 (2H, dd, J=1.6 and 12.0 Hz, ArH), 10.31 (1H, s, NHOH).

MF C₁₉H₂₅N₃O₃S, MW 375.49

### Ex 158 4-[1-(3-(S)-Amino-3-naphthalen-1-yl-propionyl)-piperidin-4-yl]-N-hydroxy-butyramide

MF C₂₂H₂₉N₃O₃, MW 383.48

### Ex 159 4-[1-(3-(R)-Amino-3-naphthalen-1-yl-propionyl)-piperidin-4-yl]-N-hydroxy-butyramide

¹H NMR (DMSO-d₆, 400 MHz): δ (ppm) 0.61-0.91 (2H, m), 1.12 (1H, m), 1.34-1.65 (6H, m), 1.92 (2H, q, J=7.4 Hz), 2.51 (1H, m, N(CHH)₂), 2.86 (1H, q, J=12.2 Hz, N(CHH)₂), 2.99-3.16 (2H, m, CH₂CH₂CO), 3.70 (1H, brs, N(CHH)₂), 4.36 (1H, brs, N(CHH)₂), 5.48 (1H, s, CHNH₂), 7.54-7.74 (4H, m, ArH), 8.00 (2H, t, J=9.4 Hz, ArH), 8.13 (1H, d, J=8.4 Hz, ArH), 8.45-8.50 (2H, bs, OH, NH⁺), 10.32 (1H, s, NHOH).

MF C₂₂H₂₉N₃O₃, MW 383.48

### Ex 160 4-(4-hydroxyamino)-4-oxobutyl)-N-(1-methoxynaphthalen-2-yl)piperidine-1-carboxamide

¹H NMR (DMSO-d₆, 400 MHz): δ (ppm) 0.97-1.14 (2H, m), 1.14-1.27 (2H, m), 1.38-1.50 (1H, m) 1.53 (2H, quin, J=7.4 Hz), 1.63-1.73 (2H, m), 1.94 (2H, t, J=7.6 Hz, CH₂CO), 2.82 (2H, m, N(CHH)₂), 3.82 (3H, s, OCH₃), 4.13 (2H, m, N(CHH)₂), 7.42 (1H, t, J=7.2 Hz, ArH), 7.51 (1H, t, J=7.2 Hz, ArH), 7.61 (1H, d, J=8.8 Hz, ArH), 7.75 (1H, d, J=8.8 Hz, ArH), 7.87 (1H, d, J=8.0 Hz, ArH), 7.99 (1H, d, J=8.0 Hz, ArH), 8.05 (1H, s, NCONH), 8.66 (1H, s, OH), 10.32 (1H, s, NHOH).

MF C₂₁H₂₇N₃O₄, MW 385.46

### Ex 161 4-(4-hydroxyamino)-4-oxobutyl)-N-(3-methoxynaphthalen-2-yl)piperidine-1-carboxamide

¹H NMR (DMSO-d₆, 400 MHz): δ (ppm) 0.99-1.12 (2H, m), 1.14-1.25 (2H, m), 1.37-1.48 (1H, m) 1.53 (2H, quin, J=7.6 Hz), 1.65-1.74 (2H, m), 1.94 (2H, t, J=7.6 Hz, CH₂CO), 2.81 (2H, m, N(CHH)₂), 3.96 (3H, s, OCH₃), 4.05 (2H, m, N(CHH)₂), 7.25-7.36 (3H, m, ArH), 7.66-7.79 (2H, m, ArH), 8.25-8.29 (1H, m, ArH), 8.66 (1H, s, OH), 10.32 (1H, s, NHOH).

MF C₂₁H₂₇N₃O₄, MW 385.46

### Ex 162 4-(4-hydroxyamino)-4-oxobutyl)-N-((5-methyl-2-phenyloxazol-4-yl)methyl)piperidine-1-carboxamide

¹H NMR (DMSO-d₆, 400 MHz): δ (ppm) 0.91 (2H, dq, J=3.6 and 11.6 Hz), 1.08-1.20 (2H, m), 1.27-1.41 (1H, m) 1.49 (2H, quin, J=7.6 Hz), 1.53-1.62 (2H, m), 1.91 (2H, t, J=7.2 Hz, CH₂CO), 2.37 (3H, s, CH₃), 2.60 (2H, m, N(CHH)₂), 3.94 (2H, m, N(CHH)₂), 4.09 (2H, m, PhCH₂), 6.87 (1H, t, J=5.6 Hz, NCONH), 7.43-7.55 (3H, m, ArH), 7.85-7.95 (2H, m, ArH), 8.65 (1H, bs, OH), 10.30 (1H, s, NHOH).

MF C₂₁H₂₈NO₄, MW 400.47

### Ex 163 N-(2-(1H-indol-3-yl)ethyl)-4-(4-(hydroxyamino)-4-oxobutyl)piperidine-1-carboxamide

¹H NMR (DMSO-d₆, 400 MHz): δ (ppm) 0.84-0.99 (2H, m), 1.11-1.21 (2H, m), 1.28-1.41 (1H, m) 1.50 (2H, quin, J=7.6 Hz), 1.54-1.62 (2H, m), 1.92 (2H, t, J=7.6 Hz, CH₂CO), 2.60 (2H, m, N(CHH)₂), 2.80 (2H, J=7.6 Hz, NHCH₂CH₂), 3.26 (2H, J=7.6 Hz, NHCH₂CH₂), 3.92 (2H, m, N(CHH)₂), 6.96 (1H, t, J=7.4 Hz, ArH), 7.05 (1H, t, J=7.4 Hz, ArH), 7.11 (1H, s, IndH), 7.32 (1H, d, J=8.0 Hz, ArH), 7.54 (1H, d, J=8.0 Hz, ArH), 10.31 (1H, s, NHOH), 10.75 (1H, s, IndNH).

MF C₂₀H₂₈N₄O₃, MW 372.46

### Ex 164 4-[1-(3-Benzylamino-propionyl)-piperidin-4-yl]-N-hydroxy-butyramide

¹H NMR (DMSO-d₆, 400 MHz): δ (ppm) 0.75-1.30 (4H, m), 1.45-1.80 (5H, m), 1.95 (2H, t, J=7.0 Hz, CH₂CO), 2.55 (1H, t, J=11.5 Hz, CH₂NH), 2.70 (2H, t, J=6.7 Hz, CH₂CO), 2.95 (1H, t, J=11.5 Hz, CH₂NH), 3.10 (2H, t, J=6.7 Hz, CH₂NH), 3.70 (1H, d, J=11.5 Hz, CH₂NH), 4.18 (2H, s, CH₂Ph), 4.35 (1H, d, J=11.5 Hz, CH₂NH), 7.40-7.55 (5H, m, ArH), 8.60 (1H, s, NH⁺), 8.65 (1H, bs, OH), 10.35 (1H, s, NHOH).

MF C₁₉H₂₉N₃O₃, MW: 347.452

### Ex 165 4-(3-Hydroxycarbamoyl-propyl)-piperidine-1-carboxylic acid (3′-fluoro-biphenyl-4-yl)-amide

¹H NMR (DMSO-d₆, 400 MHz): δ (ppm) 1.02 (2H, dq, J=3.4 and 12.3 Hz), 1.11-1.29 (2H, m), 1.36-1.48 (1H, m), 1.48-1.59 (2H, m), 1.62-1.73 (2H, m), 1.94 (2H, t, J=7.3 Hz, CH₂CO), 2.75 (2H, m, N(CHH)₂), 4.12 (2H, m, N(CHH)₂), 7.07-7015 (1H, m, ArH), 7.41-7051 (4H, m, ArH), 7.54-7.62 (3H, m, ArH), 8.57 (1H, m, NHCON), 8.67 (1H, bs, OH), 10.32 (1H, s, NHOH).

MF C₂₂H₂₆FN₃O₃, MW 399.46

### Ex 166 4-(3-Hydroxycarbamoyl-propyl)-piperidine-1-carboxylic acid (4′-fluoro-biphenyl-4-yl)-amide

¹H NMR (DMSO-d₆, 400 MHz): δ (ppm) 1.02 (2H, m), 1.13-1.29 (2H, m), 1.37-1.47 (1H, m), 1.47-1.60 (2H, m), 1.63-1.73 (2H, m), 1.94 (2H, t, J=7.3 Hz, CH₂CO), 2.75 (2H, m, N(CHH)₂), 4.11 (2H, m, N(CHH)₂), 7.24 (2H, m, ArH), 7.53 (4H, m, ArH), 7.65 (2H, m, ArH), 8.53 (1H, m, NHCON), 8.65 (1H, bs, OH), 10.32 (1H, s, NHOH).

MF C₂₂H₂₆FN₃O₃, MW 399.46

### Ex 167 4-{1-[3-(S)-Amino-3-(2-chloro-phenyl)-propionyl]-piperidin-4-yl}-N-hydroxy-butyramide

MF C₁₈H₂₆ClN₃O₃, MW 367.87

### Ex 168 4-{1-[3-(S)-Amino-3-(3-chloro-phenyl)-propionyl]-piperidin-4-yl}-N-hydroxy-butyramide

¹H NMR (DMSO-d₆, 400 MHz): δ (ppm) 0.84-0.96 (2H, m), 1.13 (2H, m), 1.47 (3H, brs), 1.64 (2H, m), 1.92 (2H, brs), 2.54 (1H, m, N(CHH)₂), 2.84-3.07 (3H, m, CH₂CO, N(CHH)₂), 3.76 (1H, d, J=13.1 Hz, N(CHH)₂), 4.37 (1H, d, J=12.8 Hz, N(CHH)₂), 4.62 (1H, s, CHNH₂), 7.45 (3H, s, ArH), 7.61 (1H, s, ArH), 8.33 (1H, s, OH), 10.30 (1H, s, NH).

MF C₁₈H₂₆ClN₃O₃, MW 367.87

### Ex 169 4-(3-Hydroxycarbamoyl-propyl)-piperidine-1-carboxylic acid (3′-methoxy-biphenyl-4-yl)-amide

¹H NMR (DMSO-d₆, 400 MHz): δ (ppm) 1.02 (2H, dq, J=3.6 and 12.5 Hz), 1.15-1.26 (2H, m), 1.36-1.47 (1H, m), 1.47-1.58 (2H, m), 1.61-1.72 (2H, m), 1.94 (2H, t, J=7.3 Hz, CH₂CO), 2.74 (2H, m, N(CHH)₂), 3.77 (3H, s, OCH₃), 4.11 (2H, m, N(CHH)₂), 6.98 (2H, d, J=8.8 Hz, ArH), 7.44-7.59 (6H, m, ArH), 8.48 (1H, m, NHCON), 8.54 (1H, bs, OH), 10.32 (1H, s, NHOH).

MF C₂₃H₂₉N₃O₄, MW 411.49

### Ex 170 N-(3-(1H-indol-3-yl)propyl)-4-(4-(hydroxyamino)-4-oxobutyl)piperidine-1-carboxamide

¹H NMR (DMSO-d₆, 400 MHz): δ (ppm) 0.91 (2H, dq, J=3.2 and 12.4 Hz), 1.09-1.19 (2H, m), 1.27-1.40 (1H, m) 1.49 (2H, quint, J=7.2 Hz), 1.53-1.62 (2H, m), 1.76 (2H, quint, J=7.2 Hz, NCH₂CH₂CH₂), 1.91 (2H, t, J=7.2 Hz, CH₂CO), 2.57 (2H, t, J=11.6 Hz, NCH₂CH₂CH₂), 2.65 (2H, m, N(CHH)₂), 3.07 (2H, t, J=6.8 Hz, NCH₂CH₂CH₂), 3.91 (2H, m, N(CHH)₂), 6.39 (1H, s, NCONH), 6.95 (1H, t, J=7.6 Hz, ArH), 7.05 (1H, t, J=7.6 Hz, ArH), 7.11 (1H, s, ArH), 7.31 (1H, d, J=8.0 Hz, ArH), 7.47 (1H, d, J=8.0 Hz, ArH), 10.30 (1H, s, NHOH), 10.72 (1H, bs, IndNH).

MF C₂₁H₃₀N₄O₃, MW 386.49

### Ex 171 4-{1-[3-(S)-Amino-3-(3-chloro-phenyl)-propionyl]-piperidin-4-yl}-N-hydroxy-butyramide

MF C₁₈H₂₆ClN₃O₃, MW 367.87

### Ex 172 N-Hydroxy-4-[1-(4-methoxy-benzylthiocarbamoyl)-piperidin-4-yl]-butyramide

¹H NMR (DMSO-d₆, 400 MHz): δ (ppm) 0.99 (2H, dq, J=3.6 and 12.9 Hz), 1.11-1.26 (2H, m), 1.46-1.54 (3H, m), 1.60-1.70 (2H, m), 1.93 (2H, t, J=7.3 Hz, CH₂CO), 2.91 (2H, m, N(CHH)₂), 3.81 (3H, s, OCH₃), 4.69 (2H, m, N(CHH)₂), 4.77 (2H, s, CH₂Ph), 7.86 (2H, d, J=8.6 Hz, ArH), 7.20 (2H, d, J=8.6 Hz, ArH), 8.03 (1H, m, NHCON), 8.66 (1H, bs, OH), 10.31 (1H, s, NHOH).

MF C₁₈H₂₇N₃O₃S, MW 365.49

### Ex 173 4-(1-Benzylthiocarbamoyl-piperidin-4-yl)-N-hydroxy-butyramide

¹H NMR (DMSO-d₆, 400 MHz): δ (ppm) 1.01 (2H, dq, J=3.4 and 13.0 Hz), 1.14-1.20 (2H, m), 1.42-1.54 (1H, m), 1.62-1.75 (2H, m), 1.93 (2H, t, J=7.3 Hz, CH₂CO), 2.77 (2H, m, N(CHH)₂), 4.65 (2H, m, N(CHH)₂), 4.77 (2H, s, CH₂Ph), 7.17-7.34 (5H, m, ArH), 8.11 (1H, m, NHCON), 8.65 (1H, s, OH), 10.31 (1H, s, NHOH).

MF C₁₇H₂₅N₃O₂S, MW 335.46

### Ex 174 4-{1-[3-(S)-Amino-3-(4-fluoro-phenyl)-propionyl]-piperidin-4-yl}-N-hydroxy-butyramide

MF C₁₈H₂₆FN₃O₃ MW 351.42

### Ex 175 4-{1-[3-(S)-Amino-3-(3-fluoro-phenyl)-propionyl]-piperidin-4-yl}-N-hydroxy-butyramide

MF C₁₈H₂₆FN₃O₃ MW 351.42

### Ex 176 4-{1-[3-(S)-Amino-3-(2-fluoro-phenyl)-propionyl]-piperidin-4-yl}-N-hydroxy-butyramide

¹H NMR (DMSO-d₆, 400 MHz): δ (ppm) 0.82-1.00 (2H, m), 1.14 (2H, m), 1.47 (3H, m), 1.64 (2H, m), 1.91 (2H, m), 2.53 (1H, m, N(CHH)₂), 2.89-3.06 (3H, m, CH₂CO, N(CHH)₂), 3.78 (1H, d, J=12.5 Hz, N(CHH)₂), 4.33 (1H, d, N(CHH)₂), 4.81 (1H, m, NCHCH₂), 7.24-7.61 (4H, m, ArH), 8.38 (1H, s, NH⁺), 8.64 (1H, s, OH), 10.31 (1H, s, NH).

MF C₁₈H₂₆FN₃O₃ MW 351.42

### Ex 177 N-(benzo[d]isoxazol-3-ylmethyl)-4-(4-(hydroxyamino)-4-oxobutyl)piperidine-1-carboxamide

¹H NMR (DMSO-d₆, 400 MHz): δ (ppm) 0.91 (2H, dq, J=3.2 and 12.4 Hz), 1.09-1.17 (2H, m), 1.28-1.43 (1H, m) 1.48 (2H, quint, J=8.0 Hz), 1.53-1.63 (2H, m), 1.91 (2H, t, J=7.2 Hz, CH₂CO), 2.64 (2H, m, N(CHH)₂), 3.96 (2H, m, N(CHH)₂), 4.62 (2H, s, PhCH₂), 7.25 (1H, s, NCONH), 7.37 (1H, t, J=7.2 Hz, ArH), 7.63 (1H, t, J=7.2 Hz, ArH), 7.68-7.73 (1H, m, ArH), 7.92 (1H, d, J=8.0 Hz, ArH).

MF C₁₈H₂₄N₄O₄ MW 360.41

### Ex 178 4-(3-Hydroxycarbamoyl-propyl)-piperidine-1-carboxylic acid (4-[1,2,3]thiadiazol-4-yl-phenyl)-amide

¹H NMR (DMSO-d₆, 400 MHz): δ (ppm) 1.03 (2H, dq, J=3.6 and 12.5 Hz), 1.17-1.27 (2H, m), 1.36-1.48 (1H, m), 1.53 (2H, m), 1.62-1.75 (2H, m), 1.94 (2H, t, J=7.3 Hz, CH₂CO), 2.77 (2H, m, N(CHH)₂), 4.13 (2H, m, N(CHH)₂), 7.65 (2H, d, J=8.8 Hz, ArH), 7.99 (1H, d, J=8.8 Hz, ArH), 8.70 (1H, s, NHCON), 9.40 (1H, s, ThioH), 10.32 (1H, s, NHOH).

MF C₁₈H₂₃N₅O₃S, MW 389.47

### Ex 179 4-(3-Hydroxycarbamoyl-propyl)-piperidine-1-carboxylic acid [4-(3,5-dimethyl-pyrazol-1-yl)-phenyl]-amide

¹H NMR (DMSO-d₆, 400 MHz): δ (ppm) 1.02 (2H, dq, J=3.6 and 12.6 Hz), 1.14-1.28 (2H, m), 1.37-1.46 (1H, m), 1.53 (2H, quin, J=7.6 Hz), 1.62-1.73 (2H, m), 1.94 (2H, t, J=7.3 Hz, CH₂CO), 2.16 (3H, s, CH₃), 2.23 (3H, s, CH₃), 2.76 (2H, m, N(CHH)₂), 4.11 (2H, m, N(CHH)₂), 6.02 (1H, s, PyrH), 7.30 (2H, d, J=8.9 Hz, ArH), 7.55 (2H, d, J=8.9 Hz, ArH), 8.60 (1H, s, NHCON), 10.31 (1H, s, NHOH).

MF C₂₁H₂₉N₅O₃, MW 399.4

### Ex 180 4-(3-Hydroxycarbamoyl-propyl)-piperidine-1-carboxylic acid [3-(2-methyl-thiazol-4-yl)-phenyl]-amide

¹H NMR (DMSO-d₆, 400 MHz): δ (ppm) 1.02 (2H, dq, J=3.5 and 12.3 Hz), 1.14-1.26 (2H, m), 1.38-1.48 (1H, m), 1.52 (2H, quin, J=7.4 Hz), 1.61-1.71 (2H, m), 1.94 (2H, t, J=7.3 Hz, CH₂CO), 2.71 (3H, s, CH₃), 2.74 (2H, m, N(CHH)₂), 4.12 (2H, m, N(CHH)₂), 7.25 (1H, t, J=7.9 Hz, ArH), 7.46 (1H, t, J=7.4 Hz, ArH), 8.02 (1H, s, ArH), 8.54 (1H, s, ThiazH), 8.54 (1H, s, NHCON), 10.31 (1H, s, NHOH).

MF C₂₀H₂₆N₄O₃S, MW 402.51

### Ex 181 N-(6-aminonaphthalen-2-yl)-4-(4-(hydroxyamino)-4-oxobutyl)piperidine-1-carboxamide

¹H NMR (DMSO-d₆, 400 MHz): δ (ppm) 1.03 (2H, dq, J=3.2 and 12.4 Hz), 1.12-1.25 (2H, m), 1.35-1.48 (1H, m) 1.53 (2H, quint, J=7.6 Hz), 1.63-1.73 (2H, m), 1.94 (2H, t, J=7.2 Hz, CH₂CO), 2.77 (2H, m, N(CHH)₂), 4.14 (2H, m, N(CHH)₂), 7.15-7.22 (1H, m, ArH), 7.39 (1H, s, ArH), 7.55-7.61 (1H, m, ArH), 7.71 (2H, t, J=9.6 Hz, ArH), 7.96 (1H, s, ArH), 8.61 (1H, s, NCONH), 10.32 (1H, s, NHOH).

MF C₂₀H₂₆N₄O₃, MW 370.45

### Ex 182 4-(4-(hydroxyamino)-4-oxobutyl)-N-(1H-indol-5-yl)piperidine-1-carboxamide

¹H NMR (DMSO-d₆, 400 MHz): δ (ppm) 1.02 (2H, dq, J=3.2 and 12.4 Hz), 1.12-1.26 (2H, m), 1.35-1.47 (1H, m) 1.53 (2H, quint, J=8.0 Hz), 1.60-1.71 (2H, m), 1.94 (2H, t, J=7.2 Hz, CH₂CO), 2.71 (2H, m, N(CHH)₂), 4.10 (2H, m, N(CHH)₂), 6.30 (1H, m, ArH), 7.09 (1H, dd, J=2.0 and 8.8 Hz, ArH), 7.21 (1H, s, ArH), 7.22-7.26 (1H, m, ArH), 7.56 (1H, s, ArH), 8.18 (1H, s, NCONH), 8.66 (1H, s, OH), 10.32 (1H, s, NHOH), 10.86 (1H, bs, IndNH).

MF C₁₈H₂₄N₄O₃, MW 344.41

### Ex 183 4-(3-Hydroxycarbamoyl-propyl)-piperidine-1-carboxylic acid (5-methyl-1-phenyl-1H-pyrazol-4-yl)-amide

¹H NMR (DMSO-d₆, 400 MHz): δ (ppm) 1.01 (2H, dq, J=3.7 and 12.6 Hz), 1.17-1.21 (2H, m), 1.35-1.46 (1H, m), 1.52 (2H, m), 1.59-1.69 (2H, m), 1.94 (2H, t, J=7.3 Hz, CH₂CO), 2.17 (3H, s, CH₃), 2.73 (2H, m, N(CHH)₂), 4.06 (2H, m, N(CHH)₂), 7.37-7.42 (1H, m, PyrH), 7.47-7.55 (5H, m, ArH), 8.50 (1H, s, NHCON), 10.32 (1H, s, NHOH).

MF C₂₀H₂₇N₅O₃, MW 385.46

### Ex 184 4-(3-Hydroxycarbamoyl-propyl)-piperidine-1-carboxylic acid (4-pyrrol-1-yl-phenyl)-amide

¹H NMR (DMSO-d₆, 400 MHz): δ (ppm) 1.02 (2H, dq, J=3.9 and 12.7 Hz), 1.16-1.22 (2H, m), 1.37-1.47 (1H, m), 1.52 (2H, m), 1.62-1.68 (2H, m), 1.94 (2H, t, J=7.3 Hz, CH₂CO), 2.74 (2H, m, N(CHH)₂), 4.10 (2H, m, N(CHH)₂), 6.21 (2H, t, J=2.1 Hz, PyrH), 7.25 (2H, t, J=2.1 Hz, PyrH), 7.40 (2H, d, J=9.0 Hz, ArH), 7.53 (2H, d, J=9.0 Hz, ArH), 8.50 (1H, s, OH), 10.32 (1H, s, NH).

MF C₂₀H₂₆N₄O₃, MW 370.44

### Ex 185 4-(3-Hydroxycarbamoyl-propyl)-piperidine-1-carboxylic acid (3-pyrrol-1-yl-phenyl)-amide

¹H NMR (DMSO-d₆, 400 MHz): δ (ppm) 1.02 (2H, dq, J=3.9 and 12.8 Hz), 1.16-1.23 (2H, m), 1.38-1.47 (1H, m), 1.52 (2H, quin, J=7.4 Hz), 1.63-1.71 (2H, m), 1.94 (2H, t, J=7.3 Hz, CH₂CO), 2.75 (2H, m, N(CHH)₂), 4.11 (2H, m, N(CHH)₂), 6.25 (2H, t, J=2.1 Hz, PyrH), 7.09 (1H, dd, J=1.4 and 7.8 Hz, ArH), 7.22 (2H, t, J=2.1 Hz, PyrH), 7.28 (1H, t, J=8.1 Hz, ArH), 7.33-7.38 (1H, m, ArH), 7.69 (1H, t, J=2.0 Hz, ArH), 8.57 (1H, s, OH), 10.32 (1H, s, NH).

MF C₂₀H₂₆N₄O₃, MW 370.44

### Ex 186 4-[1-(3-(1)Naphtylamino-propionyl)-piperidin-4-yl]-N-hydroxy-butyramide

¹H NMR (DMSO-d₆, 400 MHz): δ (ppm) 0.75-1.30 (4H, m), 1.45-1.80 (5H, m), 1.95 (2H, t, J=7.1 Hz, CH₂CO), 2.55 (1H, t, J=13.0 Hz, CHHNH), 2.76 (2H, t, J=6.8 Hz, CH₂CO), 2.95 (1H, t, J=13.0 Hz, CHHNH), 3.25 (2H, t, J=6.8 Hz, CH₂NH), 3.75 (1H, d, J=13.0 Hz, CHHNH), 4.35 (1H, d, J=13.0 Hz, CHHNH), 4.70 (2H, s, CH₂Ph), 7.55-7.75 (4H, m, ArH), 8.03 (2H, d, J=8.1 Hz, ArH), 8.24 (1H, d, J=8.1 Hz, ArH), 8.60 (1H, s, NH⁺), 8.65 (1H, bs, OH), 10.32 (1H, s, NHOH).

MF C₂₃H₃₁N₃O₃, MW: 397.51

### Ex 187 4-(4-(hydroxyamino)-4-oxobutyl)-N-(1H-indol-3-yl)piperidine-1-carboxamide

¹H NMR (DMSO-d₆, 400 MHz): δ (ppm) 1.04 (2H, dq, J=3.2 and 12.4 Hz), 1.13-1.26 (2H, m), 1.35-1.47 (1H, m) 1.53 (2H, quint, J=7.6 Hz), 1.62-1.72 (2H, m), 1.94 (2H, t, J=7.2 Hz, CH₂CO), 2.74 (2H, m, N(CHH)₂), 4.13 (2H, m, N(CHH)₂), 6.93 (1H, t, J=7.6 Hz, ArH), 7.04 (1H, t, J=7.6 Hz, ArH), 7.28 (1H, d, J=8.0 Hz, ArH), 7.33 (1H, s, ArH), 7.58 (1H, d, J=8.0 Hz, ArH), 8.08 (1H, t, J=5.6 Hz, NCONH), 8.65 (1H, s, OH), 10.32 (1H, s, NHOH), 10.64 (1H, bs, IndNH).

MF C₁₈H₂₄N₄O₃, MW 344.41

### Ex 188 N-(5-Chloro-benzo[b]thiophen-3-ylmethyl)-4-(4-(hydroxyamino)-4-oxobutyl)piperidine-1-carboxamide

¹H NMR (DMSO-d₆, 400 MHz): δ (ppm) 0.92 (2H, dq, J=3.2 and 12.0 Hz), 1.10-1.20 (2H, m), 1.28-1.42 (1H, m) 1.49 (2H, quint, J=8.0 Hz), 1.53-1.62 (2H, m), 1.91 (2H, t, J=7.2 Hz, CH₂CO), 2.63 (2H, m, N(CHH)₂), 3.95 (2H, m, N(CHH)₂), 4.43 (2H, s, PhCH₂), 7.10 (1H, t, J=5.6 Hz, NCONH), 7.39 (1H, dd, J=2.0 and 8.4 Hz, ArH), 7.55 (1H, s, ArH), 7.97-8.04 (2H, d, J=5.2 Hz, ArH), 10.30 (1H, s, NHOH).

MF C₁₉H₂₄ClN₃O₃S, MW 409.93

### Ex 189 N-(benzo[b]thiophen-5-yl)-4-(4-(hydroxyamino)-4-oxobutyl)piperidine-1-carboxamide

¹H NMR (DMSO-d₆, 400 MHz): δ (ppm) 1.03 (2H, dq, J=3.2 and 11.6 Hz), 1.15-1.26 (2H, m), 1.35-1.47 (1H, m) 1.53 (2H, quint, J=8.0 Hz), 1.61-1.71 (2H, m), 1.94 (2H, t, J=7.2 Hz, CH₂CO), 2.75 (2H, m, N(CHH)₂), 4.13 (2H, m, N(CHH)₂), 7.34 (1H, d, J=5.2 Hz, ArH), 7.42 (1H, dd, J=2.0 and 8.8 Hz, ArH), 7.67 (1H, d, J=5.2 Hz, ArH), 7.81 (1H, d, J=8.8 Hz, ArH), 8.02 (1H, d, J=2.0 Hz, ArH), 8.52 (1H, m, NCONH), 8.66 (1H, s, OH), 10.32 (1H, s, NHOH).

MF C₁₈H₂₃N₃O₃S, MW 361.46

### Ex 190 N-(4-(thiophen-3-yl)benzyl)-4-(4-(hydroxyamino)-4-oxobutyl)piperidine-1-carboxamide

¹H NMR (DMSO-d₆, 400 MHz): δ (ppm) 0.94 (2H, dq, J=3.2 and 12.0 Hz), 1.11-1.21 (2H, m), 1.30-1.43 (1H, m) 1.50 (2H, quint, J=8.0 Hz), 1.55-1.64 (2H, m), 1.92 (2H, t, J=7.2 Hz, CH₂CO), 2.64 (2H, m, N(CHH)₂), 3.97 (2H, m, N(CHH)₂), 4.22 (2H, s, PhCH₂), 7.00 (1H, m, NCONH), 7.26 (2H, d, J=8.0 Hz, ArH), 7.51-7.55 (1H, m, ArH), 7.60-7.66 (3H, m, ArH), 7.79-7.83 (1H, m, ArH), 8.65 (1H, s, OH), 10.31 (1H, s, NHOH).

MF C₂₁H₂₇N₃O₃S, MW 401.52

### Ex 191 4-(4-(hydroxyamino)-4-oxobutyl)-N(3-phenylbenzyl)piperidine-1-carboxamide

¹H NMR (DMSO-d₆, 400 MHz): δ (ppm) 0.90 (2H, dq, J=3.6 and 12.4 Hz), 1.10-1.19 (2H, m), 1.31-1.44 (1H, m) 1.50 (2H, quint, J=7.6 Hz), 1.55-1.63 (2H, m), 1.92 (2H, t, J=7.2 Hz, CH₂CO), 2.64 (2H, m, N(CHH)₂), 3.98 (2H, m, N(CHH)₂), 4.29 (2H, s, PhCH₂), 7.04 (1H, m, NCONH), 7.23 (1H, d, J=7.4 Hz, ArH), 7.33-7.42 (2H, m, ArH), 7.43-7.54 (4H, m, ArH), 7.61 (2H, d, J=7.4 Hz, ArH), 10.31 (1H, s, NHOH).

MF C₂₃H₂₉N₃O₃, MW 395.49

### Ex 192 3-(4-(hydroxyamino)-4-oxobutyl)-N-phenylpiperidine-1-carboxamide

¹H NMR (DMSO-d₆, 400 MHz): δ (ppm) 0.95-1.29 (3H, m), 1.29-1.46 (1H, m) 1.54 (2H, quint, J=7.6 Hz), 1.58-1.68 (1H, m), 1.74-1.84 (2H, m), 1.94 (2H, t, J=7.2 Hz, CH₂CO), 2.36-2.46 (1H, m, NCHH), 2.76 (1H, m, NCHH), 3.99 (2H, m, N(CHH)₂), 6.90 (1H, t, J=7.6 Hz, ArH), 7.20 (2H, t, J=7.6 Hz, ArH), 7.43 (2H, d, J=7.6 Hz, ArH), 8.41 (1H, m, NCONH), 8.66 (1H, s, OH), 10.33 (1H, s, NHOH).

MF C₁₆H₂₃N₃O₃, MW 305.37

### Ex 193 2-(s)-Amino-N-benzyl-4-[4-(3-hydroxycarbamoyl-propyl)-piperidin-1-yl]-4-oxo-butyramide

¹H NMR (DMSO-d₆, 400 MHz): δ (ppm) 0.82-0.94 (2H, m), 1.13-1.31 (3H, m), 1.44-1.57 (2H, m), 1.65-1.70 (2H, m), 1.90-1.95 (2H, t, J=7.6 Hz), 2.60 (1H, m), 2.86-2.99 (3H, m), 3.76 (1H, d, J=14.7 Hz), 4.09-4.14 (1H, m, NCHCH₂CO), 4.32-4.40 (2H, m, NCH₂Ph), 7.24-7.35 (5H, m, ArH), 8.10 (1H, brs, NH⁺), 8.69 (1H, s, OH), 8.80 (1H, s, NHCH₂), 10.30 (1H, s, NH).

MF C₂₀H₃₀N₄O₄, MW 390.48

### Ex 194 2-(R)-Amino-N-benzyl-4-[4-(3-hydroxycarbamoyl-propyl)-piperidin-1-yl]-4-oxo-butyramide

¹H NMR (DMSO-d₆, 400 MHz): 6?(ppm) 0.82-0.94 (2H, m), 1.13-1.31 (3H, m), 1.44-1.57 (2H, m), 1.65-1.70 (2H, m), 1.90-1.95 (2H, t, J=7.6 Hz), 2.60 (1H, m), 2.86-2.99 (3H, m), 3.76 (1H, d, J=14.7 Hz), 4.09-4.14 (1H, m, NCHCH₂CO), 4.32-4.40 (2H, m, NCH₂Ph), 7.24-7.35 (5H, m, ArH), 8.10 (1H, brs, NH⁺), 8.69 (1H, s, OH), 8.80 (1H, s, NHCH₂), 10.30 (1H, s, NH).

MF C₂₀H₃₀N₄O₄, MW 390.48

### Ex 195 N-(benzo[b]thiophene)-4-(4-(hydroxyamino)-4-oxobutyl)piperidine-1-carboxamide

¹H NMR (DMSO-d₆, 400 MHz): δ (ppm) 1.02-1.26 (4H, m), 1.39-1.57 (3H, m), 1.66 (2H, d, J=12.1 Hz), 1.94 (2H, t, J=7.3 Hz), 2.80 (2H, t, J=11.9 Hz, N(CHH)₂), 4.13 (2H, d, J=13.2 Hz, N(CHH)₂), 7.34-7.41 (2H, m, ArH), 7.53 (1H, s, ArH), 7.83-7.92 (2H, m, ArH), 8.55 (1H, m, NHCON), 8.66 (1H, s, OH), 10.32 (1H, s, NHOH).

MF C₁₈H₂₃N₃O₃S, MW 361.46

### Ex 196 4-(4-(hydroxyamino)-4-oxobutyl)-N-((3-methylbenzo[b]thiophen-2-yl)methyl)piperidine-1-carboxamide

¹H NMR (DMSO-d₆, 400 MHz): δ (ppm) 0.93 (2H, dq, J=3.2 and 12.0 Hz), 1.10-1.19 (2H, m), 1.27-1.43 (1H, m) 1.49 (2H, quint, J=7.6 Hz), 1.54-1.63 (2H, m), 1.92 (2H, t, J=7.2 Hz, CH₂CO), 2.33 (3H, s, CH₃), 2.63 (2H, m, N(CHH)₂), 3.94 (2H, m, N(CHH)₂), 4.44 (2H, s, PhCH₂), 7.15 (1H, t, J=5.6 Hz, NCONH), 7.29 (1H, m, ArH), 7.34 (1H, m, ArH), 7.67 (1H, d, J=8.0 Hz, ArH), 7.85 (1H, d, J=8.0 Hz, ArH), 10.30 (1H, s, NHOH).

MF C₂₀H₂₇N₃O₃S, MW 389.51

### Ex 197 N-((2,5-dimethylthiazol-4-yl)methyl)-4-(4-(hydroxyamino)-4-oxobutyl)piperidine-1-carboxamide

¹H NMR (DMSO-d₆, 400 MHz): δ (ppm) 0.90 (2H, dq, J=3.6 and 12.0 Hz), 1.09-1.18 (2H, m), 1.27-1.39 (1H, m) 1.48 (2H, quint, J=7.6 Hz), 1.52-1.61 (2H, m), 1.91 (2H, t, J=7.2 Hz, CH₂CO), 2.35 (3H, s, CH₃), 2.46-2.52 (2H, m), 2.50 (3H, s, CH₃), 3.92 (2H, m, N(CHH)₂), 4.14 (2H, s, PhCH₂), 6.84 (1H, m, NCONH), 10.31 (1H, s, NHOH).

MF C₁₆H₂₆N₄O₃, MW 354.47

### Ex 198 4-(3-Hydroxycarbamoyl-propyl)-piperidine-1-carboxylic acid (5,6,7,8-tetrahydro-naphthalen-1-yl)-amide

¹H NMR (DMSO-d₆, 400 MHz): δ (ppm) 1.01 (2H, dq, J=3.2 and 12.4 Hz), 1.13-1.24 (2H, m), 1.35-1.46 (1H, m) 1.52 (2H, quint, J=7.6 Hz), 1.58-1.75 (6H, m), 1.93 (2H, t, J=7.2 Hz, CH₂CO), 2.50-2.59 (2H, m), 2.65-2.79 (4H, m), 4.04 (2H, m, N(CHH)₂), 6.84 (1H, dd, J=2.4 and 6.4 Hz, ArH), 6.95-7.02 (2H, m, ArH), 7.78 (1H, s, OH), 10.32 (1H, s, NHOH).

MF C₂₀H₂₉N₃O₃, MW 359.46

### Ex 199 4-(3-Hydroxycarbamoyl-propyl)-piperidine-1-carboxylic acid (4-benzyl-phenyl)-amide

¹H NMR (DMSO-d₆, 400 MHz): δ (ppm) 0.99 (2H, dq, J=3.6 and 12.4 Hz), 1.13-1.23 (2H, m), 1.32-1.46 (1H, m) 1.51 (2H, quint, J=7.6 Hz), 1.58-1.70 (2H, m), 1.93 (2H, t, J=7.2 Hz, CH₂CO), 2.71 (2H, m, N(CHH)₂), 3.84 (2H, m, Ph₂CH₂), 4.06 (2H, m, N(CHH)₂), 7.05 (2H, d, J=8.4 Hz, ArH), 7.13-7.22 (3H, m, ArH), 7.23-7.29 (2H, m, ArH), 7.34 (2H, d, J=8.4 Hz, ArH), 8.34 (1H, s, NCONH), 8.65 (1H, s, OH), 10.31 (1H, s, NHOH).

MF C₂₃H₂₉N₃O₃, MW 395.50

### Ex 200 4-(3-Hydroxycarbamoyl-propyl)-piperidine-1-carboxylic acid (4-benzyloxy-phenyl)-amide

¹H NMR (DMSO-d₆, 400 MHz): δ (ppm) 0.99 (2H, dq, J=3.6 and 12.4 Hz), 1.13-1.23 (2H, m), 1.32-1.46 (1H, m) 1.52 (2H, quint, J=7.6 Hz), 1.60-1.70 (2H, m), 1.92 (2H, t, J=7.6 Hz, CH₂CO), 2.70 (2H, m, N(CHH)₂), 4.07 (2H, m, N(CHH)₂), 5.03 (2H, m, PhCH₂O), 6.77 (2H, d, J=8.8 Hz, ArH), 7.27-7.47 (7H, m, ArH), 8.26 (1H, bs, NCONH), 8.65 (1H, s, OH), 10.32 (1H, s, NHOH).

MF C₂₃H₂₉N₃O₄, MW 411.49

### Ex 201 Benzo[b]thiophene-2-carboxylic acid {3-[4-(3-hydroxycarbamoyl-propyl)-piperidin-1-yl]-3-oxo-propyl}-amide

¹H NMR (DMSO-d₆, 400 MHz): δ (ppm) 0.84-1.05 (2H, m), 1.09-1.15 (2H, m), 1.39-1.51 (3H, m), 1.64 (2H, m), 1.91 (2H, t, J=7.3 Hz), 2.53 (1H, m, N(CHH)₂), 2.58-2.62 (2H, m, dt, J=2.8 and 7.1 Hz, NCHCH₂), 2.95 (1H, t, J=12.7 Hz, N(CHH)₂), 3.37 (2H, m, NCH₂CH₂) 3.79 (1H, d, J=13.2 Hz, N(CHH)₂), 4.35 (1H, d, J=13.1 Hz, N(CHH)₂), 7.40-8.06 (5H, m, ArH), 8.35 (1H, s, NH⁺), 8.66 (1H, s, OH), 10.30 (1H, s, NH).

MF C₂₁H₁₉N₃O₄S, MW 419.54

### Ex 202 Benzo[b]thiophene-2-carboxylic acid {4-[4-(3-hydroxycarbamoyl-propyl)-piperidin-1-yl]-4-oxo-butyl}-amide

¹H NMR (DMSO-d₆, 400 MHz): δ (ppm) 0.84-1.01 (2H, m), 1.09-1.15 (2H, m), 1.47 (3H, m), 1.64 (2H, t, J=13.6 Hz), 1.76 (2H, m, COCH₂CH₂C), 1.91 (2H, t, J=7.3 Hz), 2.36 (2H, m, COCH₂CH₂C), 2.96 (1H, t, J=12.6 Hz), 3.27 (2H, m), 3.81 (1H, d, J=14.1 Hz, N(CHH)₂), 4.36 (1H, d, J=13.3 Hz, N(CHH)₂), 7.45 (2H, m, ArH), 7.92-8.06 (3H, m, ArH), 8.73 (1H, s, NH⁺), 10.30 (1H, s, NH).

MF C₂₂H₂₉N₃O₄S, MW 431.55

### Ex 203 Benzo[b]thiophene-2-carboxylic acid {2-(S)-amino-3-[4-(3-hydroxycarbamoyl-propyl)-piperidin-1-yl]-3-oxo-propyl}-amide

¹H NMR (DMSO-d₆, 400 MHz): δ (ppm) 0.88-0.95 (2H, m), 1.13-1.24 (2H, m), 1.37-1.54 (3H, m), 1.61-1.76 (2H, m), 1.80-1.96 (2H, m), 2.59-2.66 (1H, m, N(CHH)₂), 2.99-3.11 (1H, m, N(CHH)₂), 3.51-3.63 (2H, m), 4.03 (1H, d, J=13.5 Hz, N(CHH)₂), 4.37 (1H, d, J=13.2 Hz, N(CHH)₂), 4.55 (1H, m, CHNH₂), 7.42-8.09 (5H, m, ArH), 8.26 (1H, s, NH₃⁺), 8.61 (1H, s, OH), 9.03 (1H, s, NH⁺), 10.30 (1H, d, NH).

MF C₂₁H₂₈N₄O₄S, MW 432.54

### Ex 204 2-(s)-Amino-4-[4-(3-hydroxycarbamoyl-propyl)-piperidin-1-yl]-N-naphthalen-1-ylmethyl-4-oxo-butyramide

¹H NMR (DMSO-d₆, 400 MHz): δ (ppm) 0.82-0.95 (2H, m), 1.13-1.31 (2H, m), 1.41-1.53 (3H, m), 1.66 (2H, m), 1.90-1.95 (2H, t), 2.52 (1H, m), 2.87-2.94 (3H, m), 3.71 (1H, d, J=14.0 Hz), 4.10-4.14 (1H, m, NCHCH₂CO), 4.32-4.40 (1H, t, J=11.3 Hz), 4.81 (2H, s, NCH₂Nap), 7.47-7.57 (4H, m, ArH), 7.86-8.04 (3H, m, ArH), 8.14 (1H, brs, NH₃⁺), 8.65 (1H, s, OH), 8.82 (1H, s, NHCH₂), 10.30 (1H, s, NH).

MF C₂₄H₃₂N₄O₄, MW 440.53

### Ex 205 4-(3-Hydroxycarbamoyl-propyl)-piperidine-1-carboxylic acid [4-(2-methyl-2H-tetrazol-5-yl)-phenyl]-amide

¹H NMR (DMSO-d₆, 400 MHz): δ (ppm) 0.97-1.10 (2H, m), 1.15-1.23 (2H, m), 1.36-1.48 (1H, m), 1.52 (2H, m), 1.63-1.73 (2H, m), 1.94 (2H, t, J=7.3 Hz, CH₂CO), 2.76 (2H, m, N(CHH)₂), 4.12 (2H, m, N(CHH)₂), 4.38 (3H, s, CH₃), 7.65 (2H, d, J=8.8 Hz, ArH), 7.90 (2H, d, J=8.8 Hz, ArH), 8.71 (1H, s, OH), 10.32 (1H, s, NH).

MF C₁₈H₂₅N₇O₃, MW 429.51

### Ex 206 4-(3-Hydroxycarbamoyl-propyl)-piperidine-1-carboxylic acid [4-(2-isobutyl-2H-tetrazol-5-yl)-phenyl]-amide

¹H NMR (DMSO-d₆, 400 MHz): δ (ppm) 0.92 (6H, d, J=6.7 Hz, CH(CH₃)₂), 1.02 (2H, dq, J=3.9 and 12.8 Hz), 1.16-1.22 (2H, m), 1.39-1.47 (1H, m), 1.52 (2H, quin, J=7.4 Hz), 1.63-1.71 (2H, m), 1.94 (2H, t, J=7.3 Hz, CH₂CO), 2.31 (1H, m, CH(CH₃)₂), 2.76 (2H, m, N(CHH)₂), 4.12 (2H, m, N(CHH)₂), 4.53 (2H, d, J=7.1 Hz, CH₂CH(CH₃)₂), 7.65 (2H, d, J=8.7 Hz, ArH), 7.91 (2H, d, J=8.7 Hz, ArH), 8.71 (1H, s, OH), 10.32 (1H, s, NH).

MF C₂₁H₃₁N₇O₃, MW 429.51

### Ex 207 4-(3-Hydroxycarbamoyl-propyl)-piperidine-1-carboxylic acid [4-(3-methyl-pyrazol-1-yl)-phenyl]-amide

¹H NMR (DMSO-d₆, 400 MHz): δ (ppm) 1.02 (2H, dq, J=3.7 and 12.6 Hz), 1.16-1.21 (2H, m), 1.33-1.45 (1H, m), 1.52 (2H, quint, J=7.3 Hz), 1.62-1.70 (2H, m), 1.94 (2H, t, J=7.3 Hz, CH₂CO), 2.25 (3H, s, CH₃), 2.74 (2H, m, N(CHH)₂), 4.11 (2H, m, N(CHH)₂), 6.27 (1H, d, J=2.3 Hz, PyrH), 7.53 (2H, m, ArH), 7.61 (2H, m, ArH), 8.22 (1H, d, J=2.3 Hz, PyrH), 8.52 (1H, s, OH), 10.32 (1H, s, NH).

MF C₂₀H₂₇N₅O₃, MW 385.46

### Ex 208 4-(3-Hydroxycarbamoyl-propyl)-piperidine-1-carboxylic acid [2-(3-dimethylamino-propyl)-1H-benzoimidazol-5-yl]-amide

¹H NMR (DMSO-d₆, 400 MHz): δ (ppm) 1.01 (2H, dq, J=3.2 and 12.8 Hz), 1.11-1.26 (2H, m), 1.35-1.48 (1H, m) 1.52 (2H, quint, J=7.6 Hz), 1.61-1.74 (2H, m), 1.94 (2H, t, J=7.6 Hz, CH₂CO), 2.17 (2H, quint, J=7.6 Hz, NCH₂CH₂CH₂Ar), 2.76 (2H, m, N(CHH)₂), 2.80 (6H, s, N(CH₃)₂), 3.02-3.12 (2H, m, NCH₂CH₂CH₂Ar), 3.12-3.21 (2H, m, NCH₂CH₂CH₂Ar), 4.13 (2H, m, N(CHH)₂), 7.34-7.52 (1H, bs, NCONH), 7.52-7.66 (2H, m, ArH), 7.76-8.07 (2H, m, ArH), 8.69 (1H, bs, OH), 10.34 (1H, bs, NHOH).

MF C₂₂H₃₄N₆O₃, MW 430.54

### Ex 209 4-[1-(1H-Benzoimidazol-2-yl)-piperidin-4-yl]-N-hydroxy-butyramide

¹H NMR (DMSO-d₆, 400 MHz): δ (ppm) 1.15-1.28 (4H, m), 1.49-1.58 (3H, m), 1.81 (2H, d, J=12.3 Hz), 3.26 (2H, t, J=12.3 Hz, N(CH₂)₂), 4.01 (2H, d, J=12.1 Hz, N(CH₂)₂), 7.22-7.38 (4H, m, ArH), 8.69 (1H, bs, OH), 10.34 (1H, bs, NHOH), 12.90 (1H, brs, H benzimidazol).

MF C₁₆H₂₂N₄O₂, MW 302.37

### Therapeutic Indications

Histone deacetylase inhibitors are a class of potential therapeutic or prophylactic agents for pathological states caused by abnormal gene expression, such as inflammatory disorders, diabetes, complications of diabetes, homozygotic thalassaemia, fibrosis, cirrhosis, acute promyelocytic leukaemia (APL), transplant rejection, auto-immune diseases, protozoan infections, tumors and the like. In particular they are emerging as a new class of drugs with anti-tumor activity. The connection between some tumorous pathologies, such as carcinoma of the mammary, colon and lung, and acetylation levels of nuclear chromatin has been described. Drugs able to modulate chromatin remodelling are able to inhibit tumor proliferation and could provide new instruments for treating tumor pathologies in the not too distant future. Much experimental evidence leads to the belief that the main field of application of this class of drugs could be in combined therapies. The considerable tolerability that has emerged from the first clinical trials leads to the belief that this class of molecules lends itself to combined therapy with traditional drugs such as cytotoxic drugs, or with radiotherapy treatments or with the new generation antitumor agents. In particular, the present invention also provides combinations of compounds with histone deacetylase inhibitory activity of general formula (I) with one or more chemotherapeutic compounds chosen from the group: conventional cytotoxic agents, demethylating agents, cyclin dependent kinase inhibitors, differentiating agents, signal transduction modulators, HSP-90 antagonists, proteasome inhibitors. Preferred compounds are compounds chosen from the following groups: the conventional cytotoxic agents: fludarabine, gemcitabine, decitabine, paclitaxel, carboplatin and Topo I/II inhibitors to include Etoposide, Irinotecan, Topotecan, T-128 and Anthracyclines such as Doxorubicin, Sabarubicin, Daunorubicin;

the demethylating agents (demethylation of DNA): 5-aza-2′-deoxycytidine (5-aza-dC), 5-azacytidine;
the cyclin dependent kinase inhibitors: Flavopiridol, olomoucin, roscovitin, purvalanol B, GW9499, GW5181, CGP60474, CGP74514, AG12286, AG12275, Staurosporine, UCN-01;
the differentiating agents: retinoic acid and derivatives (All Trans Retinoic Acid, ATRA), 13-cis retinoic acid (CRA), PMA (phorbol myristate acetate); the signal transduction modulators: TRAIL, imatinib mesylate, LY-294002, bortezomib;
the HSP-90 antagonists: geldanamycin and its analogues (17-AAG);
the proteasome inhibitors: lactacystine, MG132, bortezomib (Velcade™).

### Biological Activity

The activity of the compounds as histone deacetylase (HDAC) inhibitors was measured using an in vitro acetylation assay. The compounds were then evaluated as inhibitors of proliferation of human tumor cell cultures. The overall data obtained are given in the table.

### Deacetylase Activity on Nuclear Extract of Hela Cells (Human Cervical Cancer Cell)

The assay (Fluor de Lys™ kit, BioMol) is divided into two steps: in the first step the substrate which comprises an acetylated lysine residue is reacted with the nuclear extract (HeLa) containing the enzymatic activity in the presence and absence of inhibitors. In the second step a fluorogenic reagent is added which highlights the deacetylated residues. A reduction in fluorescence is obtained where there has been inhibition of the deacetylase activity. The result is finally expressed as percent inhibition relative to the control without inhibitor at a concentration of 0.1 μM.

### Evaluation of Cytotoxic Activity on Culture of Human Colon Carcinoma Cells HCT-116

Human colon carcinoma cells HCT-116 were seeded onto 96-well plates in RPMI1640 culture medium with added 10% FBS and 2 mM glutamine. 24 hours after seeding, the compounds at different concentrations are added. All the compounds are diluted in DMSO such that the final concentrations on the cultures is no greater than 0.5%. 72 hours after addition of the compounds, cell viability is measured by means of the dye Alamar Blue. The result is expressed as percent survival of the treated relative to the control, treated with carrier alone.

TABLE
Biological activity of the new compounds on histone
acetylation inhibition and on antiproliferative activity
in the human colon carcinoma cell line HCT-116

| | % inhib. | HCT-116 |
|---|---|---|
| Example | 0.1 μM | IC50 (μM) |
| Ex 7 | 59 | 0.3 |
| Ex 8 | 53 | 0.7 |
| Ex 15 | 43 | 0.84 |
| Ex 22 | 67 | 0.94 |
| Ex 34 | 64 | 0.33 |
| Ex 35 | 55 | 0.71 |
| Ex 36 | 73 | 0.83 |
| Ex 39 | 60 | 0.81 |
| Ex 45 | 79 | 0.89 |
| Ex 52 | 68 | 0.5 |
| Ex 73 | 73 | 0.84 |
| Ex 79 | 70 | 0.77 |
| Ex 82 | 62 | 0.53 |
| Ex 83 | 58 | 0.67 |
| Ex 90 | 56 | 0.45 |
| Ex 93 | 64 | 0.5 |
| Ex 101 | 64 | 0.51 |
| Ex 102 | 54 | 0.79 |
| Ex 103 | 49 | 0.20 |
| Ex 105 | 59 | 0.95 |
| Ex 106 | 52 | 0.71 |
| Ex 109 | 71 | 0.068 |
| Ex 110 | 54 | 0.26 |
| Ex 111 | 66 | 0.55 |
| Ex 112 | 55 | 0.31 |
| Ex 113 | 61 | 0.27 |
| Ex 114 | 61 | 0.34 |
| Ex 115 | 62 | 0.9 |
| Ex 122 | 42 | 0.42 |
| Ex 126 | 63 | 0.4 |
| Ex 137 | 68 | 0.15 |
| Ex 139 | 56 | 0.4 |
| Ex 144 | 69 | 0.24 |
| Ex 146 | 68 | 0.06 |
| Ex 149 | 62 | 0.13 |
| Ex 150 | 57 | 0.18 |
| Ex 151 | 50 | 0.1 |
| Ex 154 | 82 | 0.53 |
| Ex 157 | 68 | 0.6 |
| Ex 158 | 60 | 0.9 |
| Ex 159 | 79 | 0.2 |
| Ex 160 | 67 | 0.2 |
| Ex 161 | 76 | 0.03 |
| Ex 164 | 51 | 0.6 |
| Ex 165 | 64 | 0.7 |
| Ex 168 | 55 | 0.09 |
| Ex 170 | 51 | 0.2 |
| Ex 171 | 45 | 0.4 |
| Ex 175 | 70 | 0.6 |
| Ex 178 | 45 | 0.3 |
| Ex 179 | 70 | 0.5 |
| Ex 181 | 64 | 0.47 |
| Ex 186 | 81 | 0.36 |
| Ex 188 | 50 | 0.21 |
| Ex 190 | 69 | 0.48 |
| Ex 192 | 68 | 0.6 |
| Ex 193 | 50 | 0.9 |
| Ex 195 | 42 | 0.4 |
| Ex 198 | 77 | 0.4 |

1. Compounds of general formula (II) 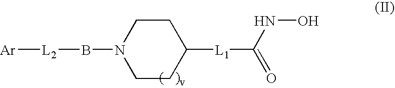
in which v=1
B is a bond, or is selected from the group consisting of —CO—, —NR5-CO—, —O—CO—, —SO₂—, —NR5-SO₂—, or represents one of the following structures: 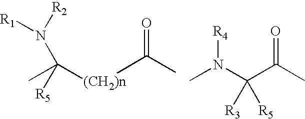
in which n=0, 1, 2
in which R5 is a H or a C1-3 alkyl
R1 represents a H or is elected from the group consisting of C1-3 alkyl, C1-3 acyl or an acyl derived from one of the following acids: benzoic, phenylacetic, benzothiophene-carboxylic, indole-carboxylic,
R2 represents a H or a C1-3 alkyl group or R1 and R2 represent jointly with the nitrogen atom, a five- or six-membered heterocycle selected from the group consisting of pyrrolidine, piperidine, piperazine, and morpholine, thiomorpholine;
R3 represents H or is from the group consisting of —C1-6 alkyl, —C1-6 alkylene-W, where W is chosen from —OR5, —SR5, —CONR7R8, —NR7R8, —OCOR6-NR5COR6, and guanidine, and R7 and R8 independently represent a H or a C1-3 alkyl group or R7 and R8 represent jointly with the nitrogen atom a five- or six-membered heterocycle selected from the group consisting of pyrrolidine, piperidine, piperazine, morpholine, and thiomorpholine, and R6 is chosen from the group hydrogen, C1-3 alkyl, Ar1 where Ar1 is an aromatic group selected from phenyl, naphthyl, pyridine, quinoline, indole, benzofuran, and benzothiophene and can possibly be substituted with up to three groups independently chosen from: C1-3 alkyl, OR9, SR9, NR9R10, N(R9)COR10, NO₂, CN, F, Cl, Br, —CF₃, COOR9, CONR9R10, CH₂NR9R10, N(R9)SO₂R10, CH₂OR9, SOH, CH₂SO₃H, in which R9 is a group chosen from H, C1-3 alkyl, —(CH₂)q-NR10R11, pyrrolidine, R10 and R11 are independently a group chosen from H and C1-3 alkyl, q can assume the values 2 or 3,
R4 represents a H or a C1-3 alkyl group
or R4 and R3 represent jointly with the nitrogen atom a heterocycle chosen from pyrrolidine or piperidine;
L1 is chosen from the group: alkylidene of the type —(CH₂)m-, possibly substituted on each C with one or two methyl groups, and in which m can assume the values 1, 2, 3
L2 indicates a bond or a group selected from the group consisting of —(CH₂)p-, —(CH₂)p-CH═CH—, —(CH₂)p-T-(CH₂)z-, —(CH₂)p-CO—, —(CH₂)p-CH═CH—CO—, —CO-T-(CH₂)z-, —(CH₂)p-T-CO—, each group being possibly substituted on the Cs with one or two methyl groups and in which p and z can independently assume the values 0, 1, 2, 3 or 4 and T is chosen from —O—, —S—, —NR5-;
Ar represents a group derived from the following aromatic systems: phenyl, pyridyl, furyl, pyrimidyl, pyrazyl, piperazyl, triazolyl, tetrazolyl, biphenyl, imidazolyl, naphthyl, quinoline, isoquinoline, diphenyl-methyl, benzofuryl, dihydrobenzofuryl, benzothienyl, indolyl, benzothiazolyl, benzoxazolyl, benzoisoxazolyl, oxazolyl-phenyl, thiodiazolyl-phenyl, pyridyl-phenyl, pyrazolyl-phenyl, thiazolyl-phenyl, furyl-phenyl, thienyl-phenyl, benzyloxy-phenyl, tetrazolyl-phenyl, phenyl-oxazolyl, phenyl-pyrazolyl-, phenyl-thiazolyl, phenyl-thiadiazolyl, phenyl-isothiazolyl, phenyl-oxadiazolyl, phenyl-isoxazolyl, phenyl-imidazolyl, phenyl-triazolyl, phenyl-furyl, phenyl-thiophenyl, phenyl-pyrrolyl, phenyl-pyrrolidyl, indanyl, fluorenyl, benzopyranyl, dihydrobenzopyranyl, benzodioxolyl, phenoxy-phenyl, benzoxazinyl, dihydrobenzoxazinyl in which each group can possibly be substituted with up to three groups independently selected from the group consisting of C1-3 alkyl, OR9, SR9, NR9R10 N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, and CH₂SO₃H, in which R9 is a group chosen from H, C1-4 alkyl, —(CH₂)q-NR10R11, and pyrrolidine, R10 and 11 are independently chosen from H and C1-3 alkyl, q can assume the values 1, 2 or 3,
or Ar-L2-B can jointly be chosen from the group consisting of: 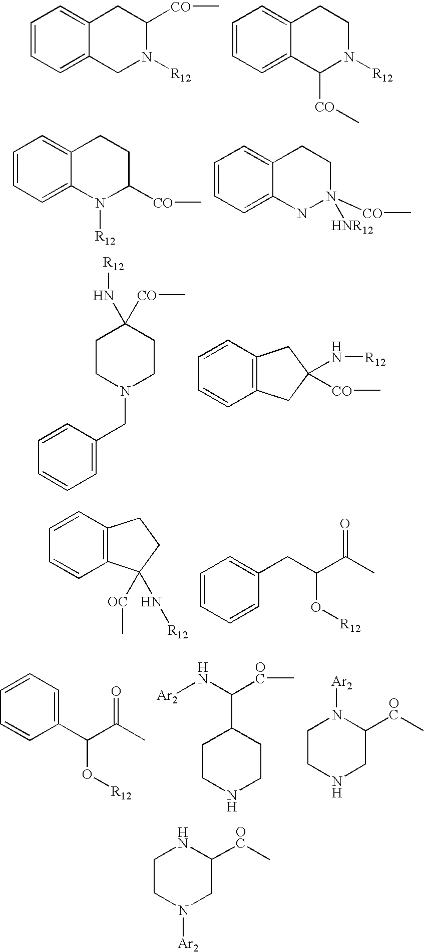
in which:
the R12 group represents a H, a C1-3 alkyl or a C1-3 acyl
the Ar2 group is an aromatic selected from the group consisting of benzyl or an acyl group derived from benzoic, phenylacetic or benzothiophene-carboxylic acids
the aromatic part can be substituted with up to three groups independently chosen from: C1-3 alkyl, OR9, SR9, NR9R10, N(R9)COR10, NO₂, CN, F, Cl, Br, —CF₃, —SCF₃, COOR9, CONR9R10, —(CH₂)q-NR9R10, N(R9)SO₂R10, CH₂OR9, SOH, CH₂SO₃H, in which R9 is a group chosen from H, C1-4 alkyl, —(CH₂)q-NR10R11, pyrrolidine, R10 and R11 are independently a group chosen from H and C1-3 alkyl, q can assume the values 1, 2 or 3;
the relative prodrugs of general formula (III) and (IV) 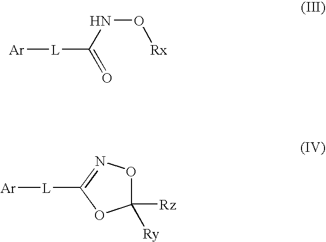
In which L is 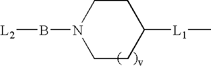
wherein Ar, L₂, B, v, and L₁ are as above defined and Rx can be Ac, COEt, CO-nPr, CO-iPr, CO-tBu, Benzoyl, pNO₂-Benzoyl, CH₃, Et, nPr, iPr, tBu, Benzyl, tetrahydropyranyl
Ry and Rz independently indicate a H or a C1-3 alkyl group;
with the exclusion of products in which:
B is chosen from —NR5-CO— or —NR5-SO₂— and at the same time
L2 is chosen from —(CH₂)p-CO— or —(CH₂)p-CH═CH—CO—;
possible optical isomers, such as enantiomers and/or diastereoisomers, their mixtures, either as racemes or in various ratios thereof, and their inorganic and organic acid salts.

2. Compounds as claimed in claim 1, of general formula (II) in which:
v=1
B is a bond, or is selected from the group consisting of —CO—, —NR5-CO—, and —O—CO—, or represents one of the following structures: 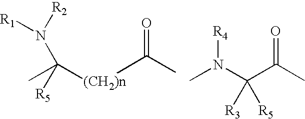
in which n=0, 1
in which R5 is a H or a C1-3 alkyl
R1 represents a H or is chosen from the group: C1-3 alkyl, C1-3 acyl
R2 represents a H or a C1-3 alkyl group
R3 represents H or is a —C1-6 alkylene-W, where W is chosen from —OR5, —NR7R8 and R7 and R8 independently represent a H or a C1-3 alkyl group —R4 represents a H or a C1-3 alkyl group,
L1 is chosen from the group: alkylidene of the type —(CH₂)m-, possibly substituted on each C with one or two methyl groups, and in which m can assume the values 2,
L2 indicates a bond or a group selected from the group consisting of —(CH₂)p-, —(CH₂)p-CH═CH—, —(CH₂)p-T-(CH₂)z-, —(CH₂)p-CO—, —CO-T-(CH₂)z-, —(CH₂)p-T-CO—, each group being possibly substituted on the Cs with one or two methyl groups and in which p and z can independently assume the values 0, 1, 2, 3, and T is
chosen from: —O—, —S—, —NR5-
Ar represents a group derived from the following aromatic systems: phenyl, pyridyl, furyl, triazolyl, biphenyl, naphthyl, quinoline, isoquinoline, benzofuryl, benzothienyl, indolyl, benzothiazolyl, benzoisoxazolyl, oxazolyl-phenyl, thiodiazolyl-phenyl, pyridyl-phenyl, pyrazolyl-phenyl, thiazolyl-phenyl, furyl-phenyl, thienyl-phenyl, benzyloxy-phenyl, tetrazolyl-phenyl, phenyl-oxazolyl, phenyl-pyrazolyl-, phenyl-thiazolyl, phenyl-thiadiazolyl, phenyl-isothiazolyl, indanyl, fluorenyl, benzodioxolyl, phenoxy-phenyl, in which each group can possibly be substituted with up to two groups independently chosen from: C1-3 alkyl, OR9, NR9R10, N(R9)COR10, CN, F, Cl, Br, —CF₃, —SCF₃, —(CH₂)q-NR9R10, in which R9 is a group chosen from H, C1-4 alkyl, R10 and 11 are independently a group chosen from H and C1-3 alkyl, q can assume the values 2 or 3,
or Ar-L2-B can jointly be chosen from the group consisting of 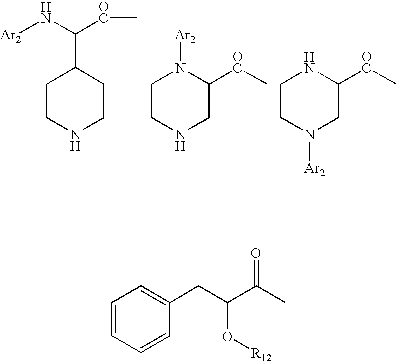
in which:
the R12 group represents a H, a C1-3 alkyl
the Ar2 group is an aromatic chosen from benzyl or an acyl group chosen from benzoic, phenylacetic or benzothiophene-carboxylic acids
the aromatic part can be substituted with up to three groups independently chosen from: a C1-3 alkyl, OR9, NR9R10, N(R9)COR10, CN, F, Cl, Br, —CF₃, —SCF₃, —(CH₂)q-NR9R10, in which R9 is a group chosen from H, C1-4 alkyl, R10 and R11 are independently a group chosen from H and C1-3 alkyl, q can assume the values 2 or 3;
with the exclusion of products in which:
B is chosen from —NR5-CO— and at the same time L2 is chosen from —(CH₂)p-CO—.

3. The compounds of general formula (II) as claimed in claim 2, namely:
4-[1-(2-Chloro-benzenesulfonyl)-piperidin-4-yl]-N-hydroxy-butyramide
N-Hydroxy-4-[1-(4-methyl-naphthalene-2-sulfonyl)-piperidin-4-yl]-butyramide
N-Hydroxy-4-[1-(naphthalene-2-carbonyl)-piperidin-4-yl]-butyramide
N-Hydroxy-4-[1-(naphthalene-1-carbonyl)-piperidin-4-yl]-butyramide
N-Hydroxy-4-[1-(2-naphthalen-1-yl-acetyl)-piperidin-4-yl]-butyramide
N-Hydroxy-4-[1-(1-naphthalen-1-yl-acetyl)-piperidin-4-yl]-butyramide
4-(3-Hydroxycarbamoyl-propyl)-piperidine-1-carboxylic acid benzylamide
4-(3-Hydroxycarbamoyl-propyl)-piperidine-1-carboxylic acid phenylamide
N-Hydroxy-4-[1-(naphthalene-2-sulfonyl)-piperidin-4-yl]-butyramide
N-Hydroxy-4-[1-(naphthalene-1-sulfonyl)-piperidin-4-yl]-butyramide
N-{3-[4-(3-Hydroxycarbamoyl-propyl)-piperidine-1-carbonyl]-phenyl}-benzamide
4-[1-(Benzofuran-2-carbonyl)-piperidin-4-yl]-N-hydroxy-butyramide
N-Hydroxy-4-[1-(6-methoxy-naphthalene-2-carbonyl)-piperidin-4-yl]-butyramide
4-(3-Hydroxycarbamoyl-propyl)-piperidine-1-carboxylic acid (3-fluoro-phenyl)-amide
N-Hydroxy-4-[1-(2-methyl-naphthalene-1-carbonyl)-piperidin-4-yl]-butyramide
4-(3-Hydroxycarbamoyl-propyl)-piperidine-1-carboxylic acid (4-bromo-phenyl)-amide
4-(3-Hydroxycarbamoyl-propyl)-piperidine-1-carboxylic acid (4-chloro-phenyl)-amide
4-(3-Hydroxycarbamoyl-propyl)-piperidine-1-carboxylic acid (3-chloro-phenyl)-amide
4-(3-Hydroxycarbamoyl-propyl)-piperidine-1-carboxylic acid (3 fluorophenyl)-amide
4-(3-Hydroxycarbamoyl-propyl)-piperidine-1-carboxylic acid (4-methoxy-phenyl)-amide
4-(3-Hydroxycarbamoyl-propyl)-piperidine-1-carboxylic acid (3-methoxy-phenyl)-amide
4-(3-Hydroxycarbamoyl-propyl)-piperidine-1-carboxylic acid p-tolylamide
4-(3-Hydroxycarbamoyl-propyl)-piperidine-1-carboxylic acid o-tolylamide
4-[1-(Benzo[b]thiophene-2-carbonyl)-piperidin-4-yl]-N-hydroxy-butyramide
4-(3-Hydroxycarbamoyl-propyl)-piperidine-1-carboxylic acid (3-fluoro-phenyl)-amide
4-[1-(Benzyloxycarbonyl)-piperidin-4-yl]-N-hydroxy-butyramide
N-Hydroxy-4-[1-(3-phenyl-acryloyl)-piperidin-4-yl]-butyramide
N-Hydroxy-4-[1-(1-methyl-1H-indole-3-carbonyl)-piperidin-4-yl]-butyramide
N-Hydroxy-4-[1-(4-phenyl-butyryl)-piperidin-4-yl]-butyramide
N-Hydroxy-4-[1-(2-1H-indol-3-yl-acetyl)-piperidin-4-yl]-butyramide
N-Hydroxy-4-{1-[2-(5-methyl-2-phenyl-oxazol-4-yl)-acetyl]-piperidin-4-yl}-butyramide
N-Hydroxy-4-{1-[2-(2-phenyl-thiazol-4-yl)-acetyl]-piperidin-4-yl}-butyramide
4-[1-(2-Benzo[d]isoxazol-3-yl-acetyl)-piperidin-4-yl]-N-hydroxy-butyramide
4-(3-Hydroxycarbamoyl-propyl)-piperidine-1-carboxylic acid 4-chloro-benzylamide
4-(3-Hydroxycarbamoyl-propyl)-piperidine-1-carboxylic acid 1-naphthylamide
4-(3-Hydroxycarbamoyl-propyl)-piperidine-1-carboxylic acid 2-naphthylamide
4-(3-Hydroxycarbamoyl-propyl)-piperidine-1-carboxylic acid (2-methoxy-phenyl)-amide
4-(3-Hydroxycarbamoyl-propyl)-piperidine-1-carboxylic acid (2-chloro-phenyl)-amide
4-(3-Hydroxycarbamoyl-propyl)-piperidine-1-carboxylic acid 3-chloro-benzylamide
4-[1-(2-(S)-Amino-3-phenyl-propionyl)-piperidin-4-yl]-N-hydroxy-butyramide
4-[1-(2-(R)-Amino-3-phenyl-propionyl)-piperidin-4-yl]-N-hydroxy-butyramide
4-(3-Hydroxycarbamoyl-propyl)-piperidine-1-carboxylic acid 2-methoxy-benzylamide
4-(3-Hydroxycarbamoyl-propyl)-piperidine-1-carboxylic acid 3-methoxy-benzylamide
4-(3-Hydroxycarbamoyl-propyl)-piperidine-1-carboxylic acid 4-methoxy-benzylamide
4-(3-Hydroxycarbamoyl-propyl)-piperidine-1-carboxylic acid 2-fluoro-benzylamide
4-(3-Hydroxycarbamoyl-propyl)-piperidine-1-carboxylic acid 3-fluoro-benzylamide
4-(3-Hydroxycarbamoyl-propyl)-piperidine-1-carboxylic acid 2-chloro-benzylamide
4-(3-Hydroxycarbamoyl-propyl)-piperidine-1-carboxylic acid (4-dimethylamino-phenyl)-amide
4-(3-Hydroxycarbamoyl-propyl)-piperidine-1-carboxylic acid (4-amino-phenyl)-amide
4-(3-Hydroxycarbamoyl-propyl)-piperidine-1-carboxylic acid 2-methyl-benzylamide
4-(3-Hydroxycarbamoyl-propyl)-piperidine-1-carboxylic acid 3-methyl-benzylamide
4-(3-Hydroxycarbamoyl-propyl)-piperidine-1-carboxylic acid 4-methyl-benzylamide
4-(3-Hydroxycarbamoyl-propyl)-piperidine-1-carboxylic acid (pyridin-4-ylmethyl)-amide
4-(3-Hydroxycarbamoyl-propyl)-piperidine-1-carboxylic acid (naphthalen-1-ylmethyl)-amide
4-(3-Hydroxycarbamoyl-propyl)-piperidine-1-carboxylic acid (naphthalen-2-ylmethyl)-amide
4-(3-Hydroxycarbamoyl-propyl)-piperidine-1-carboxylic acid (biphenyl-4-ylmethyl)-amide
4-(3-Hydroxycarbamoyl-propyl)-piperidine-1-carboxylic acid 4-phenoxy-benzylamide
4-(3-Hydroxycarbamoyl-propyl)-piperidine-1-carboxylic acid phenethyl-amide
6-{[Benzyl-(2-hydroxy-ethyl)-amino]-methyl}-7-fluoro-benzo[b]thiophene-2-carboxylic acid hydroxyamide
4-{1-[2-Amino-3-(4-chloro-phenyl)-propionyl]-piperidin-4-yl}-N-hydroxy-butyramide
4-{1-[2-Amino-3-(3-chloro-phenyl)-propionyl]-piperidin-4-yl}-N-hydroxy-butyramide MF C₁₈H₂₆ClN₃O₃, MW: 367.87
4-{1-[2-(R)-Amino-3-(3,4-dichloro-phenyl)-propionyl]-piperidin-4-yl}-N-hydroxy-butyramide.
4-{1-[2-(R)-Amino-3-(4-methoxy-phenyl)-propionyl]-piperidin-4-yl}-N-hydroxy-butyramide
4-[1-(2-(R)-Amino-2-methyl-3-phenyl-propionyl)-piperidin-4-yl]-N-hydroxy-butyramide.
4-{1-[2-(R)-Amino-3-(1H-indol-3-yl)-propionyl]-piperidin-4-yl}-N-hydroxy-butyramide
4-{1-[2-(S)-Amino-3-(1H-indol-3-yl)-propionyl]-piperidin-4-yl}-N-hydroxy-butyramide
4-[1-(2-(R)-Amino-3-benzyloxy-propionyl)-piperidin-4-yl]-N-hydroxy-butyramide
4-{1-[2-(R)-Amino-3-(4-tert-butoxy-phenyl)-propionyl]-piperidin-4-yl}-N-hydroxy-butyramide
4-[1-(2-(R)-Amino-3-benzylsulfanyl-propionyl)-piperidin-4-yl]-N-hydroxy-butyramide
4-(3-Hydroxycarbamoyl-propyl)-piperidine-1-carboxylic acid pyridin-3-ylamide
4-(3-Hydroxycarbamoyl-propyl)-piperidine-1-carboxylic acid (pyridin-3-ylmethyl)-amide
4-(3-Hydroxycarbamoyl-propyl)-piperidine-1-carboxylic acid (3-amino-phenyl)-amide
4-(3-Hydroxycarbamoyl-propyl)-piperidine-1-carboxylic acid (2-amino-phenyl)-amide
4-(3-Hydroxycarbamoyl-propyl)-piperidine-1-carboxylic acid 4-dimethylamino-benzylamide
4-(3-Hydroxycarbamoyl-propyl)-piperidine-1-carboxylic acid (4-fluoro-phenyl)-amide
4-(3-Hydroxycarbamoyl-propyl)-piperidine-1-carboxylic acid m-tolylamide
4-(3-Hydroxycarbamoyl-propyl)-piperidine-1-carboxylic acid benzyl-methyl-amide
N-Hydroxy-4-{1-[2-(2-1H-indol-3-yl-acetylamino)-acetyl]-piperidin-4-yl}-butyramide
Benzo[b]thiophene-2-carboxylic acid {2-[4-(3-hydroxycarbamoyl-propyl)-piperidin-1-yl]-2-oxo-ethyl}-amide
Naphthalene-1-carboxylic acid {2-[4-(3-hydroxycarbamoyl-propyl)-piperidin-1-yl]-2-oxo-ethyl}-amide
2-Methyl-naphthalene-1-carboxylic acid {2-[4-(3-hydroxycarbamoyl-propyl)-piperidin-1-yl]-2-oxo-ethyl}-amide
4-[1-(2-(S)-Amino-4-phenyl-butyryl)-piperidin-4-yl]-N-hydroxy-butyramide
4-(3-Hydroxycarbamoyl-propyl)-piperidine-1-carboxylic acid (3-chloro-2-fluoro-phenyl)-amide
4-(3-Hydroxycarbamoyl-propyl)-piperidine-1-carboxylic acid (2,4-difluoro-phenyl)-amide
4-(3-Hydroxycarbamoyl-propyl)-piperidine-1-carboxylic acid (3-chloro-4-methyl-phenyl)-amide
4-(3-Hydroxycarbamoyl-propyl)-piperidine-1-carboxylic acid (3,4-difluoro-phenyl)-amide
4-(3-Hydroxycarbamoyl-propyl)-piperidine-1-carboxylic acid (3,4-dimethyl-phenyl)-amide
4-(3-Hydroxycarbamoyl-propyl)-piperidine-1-carboxylic acid (3-chloro-4-fluoro-phenyl)-amide
4-(3-Hydroxycarbamoyl-propyl)-piperidine-1-carboxylic acid (2,5-difluoro-phenyl)-amide
4-(3-Hydroxycarbamoyl-propyl)-piperidine-1-carboxylic acid (4-chloro-2-fluoro-phenyl)-amide
4-(3-Hydroxycarbamoyl-propyl)-piperidine-1-carboxylic acid (2,4-dimethoxy-phenyl)-amide
N-Hydroxy-4-[1-(2-(R)-methylamino-3-phenyl-propionyl)-piperidin-4-yl]-butyramide
4-[1-(2-(R)-Amino-3-naphthalen-1-yl-propionyl)-piperidin-4-yl]-N-hydroxy-butyramide
4-[1-(2-(R)-Amino-3-benzo[b]thiophen-3-yl-propionyl)-piperidin-4-yl]-N-hydroxy-butyramide
4-[1-(2-(R)-Amino-3-m-tolyl-propionyl)-piperidin-4-yl]-N-hydroxy-butyramide
4-{1-[2-(S)-Amino-3-(4-benzoyl-phenyl)-propionyl]-piperidin-4-yl}-N-hydroxy-butyramide
4-{1-[2-(R)-Amino-3-(2-fluoro-phenyl)-propionyl]-piperidin-4-yl}-N-hydroxy-butyramide
4-{1-[2-(R)-Amino-3-(3-fluoro-phenyl)-propionyl]-piperidin-4-yl}-N-hydroxy-butyramide
4-{1-[2-(R)-Amino-3-(4-fluoro-phenyl)-propionyl]-piperidin-4-yl}-N-hydroxy-butyramide
N-Hydroxy-4-[1-(2-(R)-hydroxy-3-phenyl-propionyl)-piperidin-4-yl]-butyramide
Benzo[b]thiophene-2-carboxylic acid {2-[4-(3-hydroxycarbamoyl-propyl)-piperidin-1-yl]-1-(S)-hydroxymethyl-2-oxo-ethyl}-amide
Benzo[b]thiophene-2-carboxylic acid {2-[4-(3-hydroxycarbamoyl-propyl)-piperidin-1-yl]-1-(R)-hydroxymethyl-2-oxo-ethyl}-amide
4-[1-(2-(S)-Dimethylamino-4-phenyl-butyryl)-piperidin-4-yl]-N-hydroxy-butyramide
4-(3-Hydroxycarbamoyl-propyl)-piperidine-1-carboxylic acid 2,4-dichloro-benzylamide
4-[1-(2-(S)-Amino-4-phenyl-butyryl)-piperidin-4-yl]-N-hydroxy-butyramide
4-(3-Hydroxycarbamoyl-propyl)-piperidine-1-carboxylic acid (furan-2-ylmethyl)-amide
Benzo[b]thiophene-2-carboxylic acid{4-amino-1-(R)[4-(3-hydroxycarbamoyl-propyl)-piperidine-1-carbonyl]-butyl}-amide
4-[1-(3-(R)-Amino-4-naphthalen-2-yl-butyryl)-piperidin-4-yl]-N-hydroxy-butyramide
4-[1-(3-(S)-Amino-3-naphthalen-2-yl-propionyl)-piperidin-4-yl]-N-hydroxy-butyramide
Benzo[b]thiophene-2-carboxylic acid{3-hydroxy-1-(R)-[4-(3-hydroxycarbamoyl-propyl)-piperidine-1-carbonyl]-propyl}-amide
4-[1-(3-(R)-Amino-3-naphthalen-2-yl-propionyl)-piperidin-4-yl]-N-hydroxy-butyramide
4-[1-(3-(S)-Amino-3-phenyl-propionyl)-piperidin-4-yl]-N-hydroxy-butyramide
Benzo[b]thiophene-2-carboxylic acid{3-hydroxy-1-(S)-[4-(3-hydroxycarbamoyl-propyl)-piperidine-1-carbonyl]-propyl}-amide
Benzo[b]thiophene-5-carboxylic acid{2-[4-(3-hydroxycarbamoyl-propyl)-piperidin-1-yl]-1-(S)-hydroxymethyl-2-oxo-ethyl}-amide
Benzo[b]thiophene-2-carboxylic acid {1-(R)-aminomethyl 1-2-[4-(3-hydroxycarbamoyl-propyl)-piperidin-1-yl]-2-oxo-ethyl}-amide
4-{1-[2-(S)-(2-Benzo[b]thiophen-3-yl-acetylamino)-3-hydroxy-propionyl]-piperidin-4-yl}-N-hydroxy-butyramide
Benzo[b]thiophene-3-carboxylic acid{2-[4-(3-hydroxycarbamoyl-propyl)-piperidin-1-yl]-1-(S)-hydroxymethyl-2-oxo-ethyl}-amide
Benzofuran-2-carboxylic acid {2-[4-(3-hydroxycarbamoyl-propyl)-piperidin-1-yl]-1-(S)-hydroxymethyl-2-oxo-ethyl}-amide
N-Hydroxy-4-{1-[3-hydroxy-2-(S)-(2-naphthalen-1-yl-acetylamino)-propionyl]-piperidin-4-yl}-butyramide
1H-Indole-2-carboxylic acid {2-[4-(3-hydroxycarbamoyl-propyl)-piperidin-1-yl]-1-(S)-hydroxymethyl-2-oxo-ethyl}-amide
Quinoline-2-carboxylic acid {2-[4-(3-hydroxycarbamoyl-propyl)-piperidin-1-yl]-1-(S)-hydroxymethyl-2-oxo-ethyl}-amide
Isoquinoline-1-carboxylic acid {2-[4-(3-hydroxycarbamoyl-propyl)-piperidin-1-yl]-1-(S)-hydroxymethyl-2-oxo-ethyl}-amide
Isoquinoline-3-carboxylic acid {2-[4-(3-hydroxycarbamoyl-propyl)-piperidin-1-yl]-1-(S)-hydroxymethyl-2-oxo-ethyl}-amide
Benzo[b]thiophene-2-carboxylic acid{1-aminomethyl-2-[4-(3-hydroxycarbamoyl-propyl)-piperidin-1-yl]-2-oxo-ethyl}-amide
4-(3-Hydroxycarbamoyl-propyl)-piperidine-1-carboxylic acid 4-chloro-2-fluoro-benzylamide
4-(3-Hydroxycarbamoyl-propyl)-piperidine-1-carboxylic acid (2-amino-4-methyl-phenyl)-amide
4-(3-Hydroxycarbamoyl-propyl)-piperidine-1-carboxylic acid (2-fluoro-6-methoxy-phenyl)-amide
4-(3-Hydroxycarbamoyl-propyl)-piperidine-1-carboxylic acid (2-fluoro-5-methyl-phenyl)-amide
4-(3-Hydroxycarbamoyl-propyl)-piperidine-1-carboxylic acid (2-chloro-6-fluoro-phenyl)-amide
4-[1-(3-(R)-Amino-3-phenyl-propionyl)-piperidin-4-yl]-N-hydroxy-butyramide
4-[1-(3-(S)-Amino-5-phenyl-pentanoyl)-piperidin-4-yl]-N-hydroxy-butyramide
4-(3-Hydroxycarbamoyl-propyl)-piperidine-1-carboxylic acid (4-butyl-phenyl)-amide
N-Hydroxy-4-(1-phenylsulfamoyl-piperidin-4-yl)-butyramide
4-[1-(3-(R)-Amino-5-phenyl-pentanoyl)-piperidin-4-yl]-N-hydroxy-butyramide
4-(3-Hydroxycarbamoyl-propyl)-piperidine-1-carboxylic acid biphenyl-4-ylamide
4-(3-Hydroxycarbamoyl-propyl)-piperidine-1-carboxylic acid (benzo[1,3]dioxol-5-ylmethyl)-amide
4-(3-Hydroxycarbamoyl-propyl)-piperidine-1-carboxylic acid (4-propoxy-phenyl)-amide
4-(3-Hydroxycarbamoyl-propyl)-piperidine-1-carboxylic acid (4-phenoxyphenyl)-amide
4-(3-Hydroxycarbamoyl-propyl)-piperidine-1-carboxylic acid (4-isopropoxy-phenyl)-amide
4-(3-Hydroxycarbamoyl-propyl)-piperidine-1-carboxylic acid (1-(R)-phenyl-ethyl)-amide
4-(3-Hydroxycarbamoyl-propyl)-piperidine-1-carboxylic acid (1-(S)-phenyl-ethyl)-amide
4-[1-(3-(R)-Amino-3-naphthalen-2-yl-propionyl)-piperidin-4-yl]-N-hydroxy-butyramide
4-(3-Hydroxycarbamoyl-propyl)-piperidine-1-carboxylic acid quinolin-2-ylamide
4-[1-(3-(R)-Amino-4-naphthalen-2-yl-butyryl)-piperidin-4-yl]-N-hydroxy-butyramide
4-(3-Hydroxycarbamoyl-propyl)-piperidine-1-carboxylic acid [4-(2-diethylamino-ethyl)-phenyl]-amide
4-[1-(3-(S)-Amino-4-benzo[b]thiophen-3-yl-butyryl)-piperidin-4-yl]-N-hydroxy-butyramide
4-[1-(3-(R)-Amino-4-benzo[b]thiophen-3-yl-butyryl)-piperidin-4-yl]-N-hydroxy-butyramide
Benzo[b]thiophene-2-carboxylic acid{1-dimethylaminomethyl-2-[4-(3hydroxycarbamoyl-propyl)-piperidin-1-yl]-2-oxo-ethyl}-amide
4-[1-(3-(S)-Amino-3-naphthalen-2-yl-propionyl)-piperidin-4-yl]-N-hydroxy-butyramide
4-(3-Hydroxycarbamoyl-propyl)-piperidine-1-carboxylic acid biphenyl-3-ylamide
4-(3-Hydroxycarbamoyl-propyl)-piperidine-1-carboxylic acid (4-pyridin-2-yl-phenyl)-amide
4-(3-Hydroxycarbamoyl-propyl)-piperidine-1-carboxylic acid (4-oxazol-5-yl-phenyl)-amide
4-(3-Hydroxycarbamoyl-propyl)-piperidine-1-carboxylic acid (4-pyridin-3-yl-phenyl)-amide
4-[1-(3-(S)-Amino-4-naphthalen-1-yl-butyryl)-piperidin-4-yl]-N-hydroxy-butyramide
4-[1-(3-(R)-Amino-4-naphthalen-1-yl-butyryl)-piperidin-4-yl]-N-hydroxy-butyramide
4-(4-(hydroxyamino)-4-oxobutyl)-N-((2-phenylthiazol-4-yl)methyl)piperidine-1-carboxamide
N-(benzo[b]thiophen-3-ylmethyl)-4-(4-(hydroxyamino)-4-oxobutyl)piperidine-1-carboxamide
4-[1-(3-(S)-Amino-3-naphthalen-1-yl-propionyl)-piperidin-4-yl]-N-hydroxy-butyramide
4-[1-(3-(R)-Amino-3-naphthalen-1-yl-propionyl)-piperidin-4-yl]-N-hydroxy-butyramide
4-(4-hydroxyamino)-4-oxobutyl)-N-(1-methoxynaphthalen-2-yl)piperidine-1-carboxamide
4-(4-hydroxyamino)-4-oxobutyl)-N-(3-methoxynaphthalen-2-yl)piperidine-1-carboxamide
4-(4-hydroxyamino)-4-oxobutyl)-N-((5-methyl-2-phenyloxazol-4-yl)methyl)piperidine-1-carboxamide
N-(2-(1H-indol-3-yl)ethyl)-4-(4-(hydroxyamino)-4-oxobutyl)piperidine-1-carboxamide
4-[1-(3-Benzylamino-propionyl)-piperidin-4-yl]-N-hydroxy-butyramide
4-(3-Hydroxycarbamoyl-propyl)-piperidine-1-carboxylic acid (3′-fluoro-biphenyl-4-yl)-amide
4-(3-Hydroxycarbamoyl-propyl)-piperidine-1-carboxylic acid (4′-fluoro-biphenyl-4-yl)-amide
4-{1-[3-(S)-Amino-3-(2-chloro-phenyl)-propionyl]-piperidin-4-yl}-N-hydroxy-butyramide
4-{1-[3-(S)-Amino-3-(3-chloro-phenyl)-propionyl]-piperidin-4-yl}-N-hydroxy-butyramide
4-(3-Hydroxycarbamoyl-propyl)-piperidine-1-carboxylic acid (3′-methoxy-biphenyl-4-yl)-amide
N-(3-(1H-indol-3-yl)propyl)-4-(4-(hydroxyamino)-4-oxobutyl)piperidine-1-carboxamide
4-{1-[3-(S)-Amino-3-(3-chloro-phenyl)-propionyl]-piperidin-4-yl}-N-hydroxy-butyramide
N-Hydroxy-4-[1-(4-methoxy-benzylthiocarbamoyl)-piperidin-4-yl]-butyramide
4-(1-Benzylthiocarbamoyl-piperidin-4-yl)-N-hydroxy-butyramide
4-{1-[3-(S)-Amino-3-(4-fluoro-phenyl)-propionyl]-piperidin-4-yl}-N-hydroxy-butyramide
4-{1-[3-(S)-Amino-3-(3-fluoro-phenyl)-propionyl]-piperidin-4-yl}-N-hydroxy-butyramide
4-{1-[3-(S)-Amino-3-(2-fluoro-phenyl)-propionyl]-piperidin-4-yl}-N-hydroxy-butyramide
N-(benzo[d]isoxazol-3-ylmethyl)-4-(4-(hydroxyamino)-4-oxobutyl)piperidine-1-carboxamide
4-(3-Hydroxycarbamoyl-propyl)-piperidine-1-carboxylic acid (4-[1,2,3]thiadiazol-4-yl-phenyl)-amide
4-(3-Hydroxycarbamoyl-propyl)-piperidine-1-carboxylic acid [4-(3,5-dimethyl-pyrazol-1-yl)-phenyl]-amide
4-(3-Hydroxycarbamoyl-propyl)-piperidine-1-carboxylic acid [3-(2-methyl-thiazol-4-yl)-phenyl]-amide
N-(6-aminonaphthalen-2-yl)-4-(4-(hydroxyamino)-4-oxobutyl)piperidine-1 carboxamide
4-(4-(hydroxyamino)-4-oxobutyl)-N-(1H-indol-5-yl)piperidine-1-carboxamide
4-(3-Hydroxycarbamoyl-propyl)-piperidine-1-carboxylic acid (5-methyl-1-phenyl-1H-pyrazol-4-yl)-amide
4-(3-Hydroxycarbamoyl-propyl)-piperidine-1-carboxylic acid (4-pyrrol-1-yl-phenyl)-amide
4-(3-Hydroxycarbamoyl-propyl)-piperidine-1-carboxylic acid (3-pyrrol-1-yl-phenyl)-amide
4-[1-(3-(1)Naphtylamino-propionyl)-piperidin-4-yl]-N-hydroxy-butyramide
4-(4-(hydroxyamino)-4-oxobutyl)-N-(1H-indol-3-yl)piperidine-1-carboxamide
N-(5-Chloro-benzo[b]thiophen-3-ylmethyl)-4-(4-(hydroxyamino)-4-oxobutyl)piperidine-1-carboxamide
N-(benzo[b]thiophen-5-yl)-4-(4-(hydroxyamino)-4-oxobutyl)piperidine-1-carboxamide
N-(4-(thiophen-3-yl)benzyl)-4-(4-(hydroxyamino)-4-oxobutyl)piperidine-1-carboxamide
4-(4-(hydroxyamino)-4-oxobutyl)-N(3-phenylbenzyl)piperidine-1-carboxamide
3-(4-(hydroxyamino)-4-oxobutyl)-N-phenylpiperidine-1-carboxamide
2-(s)-Amino-N-benzyl-4-[4-(3-hydroxycarbamoyl-propyl)-piperidin-1-yl]-4-oxo-butyramide
2-(R)-Amino-N-benzyl-4-[4-(3-hydroxycarbamoyl-propyl)-piperidin-1-yl]-4-oxo-butyramide
N-(benzo[b]thiophene)-4-(4-(hydroxyamino)-4-oxobutyl)piperidine-1-carboxamide
4-(4-(hydroxyamino)-4-oxobutyl)-N-((3-methylbenzo[b]thiophen-2-yl)methyl)piperidine-1-carboxamide
N-((2,5-dimethylthiazol-4-yl)methyl)-4-(4-(hydroxyamino)-4-oxobutyl)piperidine-1-carboxamide
4-(3-Hydroxycarbamoyl-propyl)-piperidine-1-carboxylic acid (5,6,7,8-tetrahydro-naphthalen-1-yl)-amide
4-(3-Hydroxycarbamoyl-propyl)-piperidine-1-carboxylic acid (4-benzyl-phenyl)-amide
4-(3-Hydroxycarbamoyl-propyl)-piperidine-1-carboxylic acid (4-benzyloxy-phenyl)-amide
Benzo[b]thiophene-2-carboxylic acid {3-[4-(3-hydroxycarbamoyl-propyl)-piperidin-1-yl]-3-oxo-propyl}-amide
Benzo[b]thiophene-2-carboxylic acid {4-[4-(3-hydroxycarbamoyl-propyl)-piperidin-1-yl]-4-oxo-butyl}-amide
Benzo[b]thiophene-2-carboxylic acid {2-(S)-amino-3-[4-(3-hydroxycarbamoyl-propyl)-piperidin-1-yl]-3-oxo-propyl}-amide
2-(s)-Amino-4-[4-(3-hydroxycarbamoyl-propyl)-piperidin-1-yl]-N-naphthalen-1-ylmethyl-4-oxo-butyramide
4-(3-Hydroxycarbamoyl-propyl)-piperidine-1-carboxylic acid [4-(2-methyl-2H-tetrazol-5-yl)-phenyl]-amide
4-(3-Hydroxycarbamoyl-propyl)-piperidine-1-carboxylic acid [4-(2-isobutyl-2H-tetrazol-5-yl)-phenyl]-amide
4-(3-Hydroxycarbamoyl-propyl)-piperidine-1-carboxylic acid [4-(3-methyl-pyrazol-1-yl)-phenyl]-amide
4-(3-Hydroxycarbamoyl-propyl)-piperidine-1-carboxylic acid [2-(3-dimethylamino-propyl)-1H-benzoimidazol-5-yl]-amide
4-[1-(1H-Benzoimidazol-2-yl)-piperidin-4-yl]-N-hydroxy-butyramide.

4-11. (canceled)

12. A method for treating inflammatory disorders, diabetes, complications of diabetes, homozygotic thalassaemia, fibrosis, cirrhosis, acute promyelocytic leukaemia (APL), transplant rejection, auto-immune diseases, protozoan infections and tumorous pathologies wherein compounds according to claim 1 are subministered to the patient.

13. A method for treating tumorous pathologies wherein compounds according to claim 1 are subministered to the patient.

14. A method for treating tumorous pathologies wherein compounds according to claim 1 are subministered to the patient in combination with one or more active principles chosen from chemotherapeutic agents.

15. A method for treating tumorous pathologies wherein compounds according to claim 1 are subministered to the patient in combination with radiotherapeutic treatments.

16. A method for treating tumorous pathologies, wherein said compounds according to claim 1 are subministered to the patient in combination with one or more compounds selected from the group consisting of: conventional cytotoxic agents, demethylating agents, cyclin dependent kinase inhibitors, differentiating agents, signal transduction modulators, HSP-90 antagonists, proteasome inhibitors, in combination with radiotherapeutic treatments.

17. A method according to claim 16, wherein said conventional cytotoxic agents are selected from the group consisting of Fludarabine, gemcitabine, decitabine, paclitaxel, carboplatin and Topo I/II inhibitors, to include Etoposide, Irinotecan, Topotecan, T-128 and Anthracyclines such as Doxorubicin, Sabarubicin, Daunorubicin; the demethylating agents: 5-aza-2′-deoxycytidine (5-aza-dC), 5-azacytidine; the cyclin dependent kinase inhibitors: flavopiridol, olomoucin, roscovitin, purvalanol B, GW9499, GW5181, CGP60474, CGP74514, AG12286, AG12275, Staurosporine, UCN-01; the differentiating agents: retinoic acid and derivatives (All Trans Retinoic Acid, ATRA), 13-cis retinoic acid (CRA), PMA (phorbol myristate acetate); the signal transduction modulators: TRAIL, imatinib mesylate, LY-294002, bortezomib; the HSP-90 antagonists: geldanamycin and its analogues (17-AAG); the proteasome inhibitors: lactacystine, MG132, bortezomib (Velcade™).
